(19) United States
(12) Patent Application Publication
(10) Pub. No.: US 2009/0227612 A1
(43) Pub. Date: Sep. 10, 2009

(54) Aminopyrimidine Derivatives Inhibiting Protein Kinase Activity, Method For The Preparation Thereof And Pharmaceutical Composition Containing Same

(76) Inventors: Boonsaeng Park, Seoul (KR); Mi Jung Lee, Seoul (KR); Yu-Mi Song, Seoul (KR); Do Young Lee, Incheon (KR); Seung Chul Lee, Seoul (KR); Cheol Min Kim, Gyeonggi-do (KR); Seoggu Ro, Gyeonggi-do (KR); Joeng Myung Cho, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 12/158,109

(22) PCT Filed: Dec. 22, 2006
(86) PCT No.: PCT/KR2006/005661

§ 371 Date: Jun. 19, 2008

Related U.S. Application Data
(60) Provisional application No. 60/847,722, filed on Sep. 27, 2006.

(30) Foreign Application Priority Data
Dec. 22, 2005 (KR) 10-2005-0127410

(51) Int. Cl. A61K 31/505; C07D 239/32; A61P 35/00

(52) U.S. Cl. 514/275; 544/297

(57) ABSTRACT

A compound of formula 1 efficiently inhibits several protein kinases including glycogen synthase kinase 3 (GSK), aurora kinase, extracellular signal-regulated kinase (ERK), protein kinase B (AKT), and the likes, to control signal transductions involved in variable disorders such as diabetes, obesity, dementia, cancer, and inflammation.

### FIELD OF THE INVENTION

The present invention relates to a novel compound which inhibits protein kinase activity, a method for the preparation thereof, and a pharmaceutical composition comprising the same as an active ingredient.

### BACKGROUND OF THE INVENTION

Protein kinases are enzymes mediating intracellular signal transduction by delivering phosphoryl group derived from nucleoside triphosphate (NTP) to specific proteins to phosphorylate them. Many protein kinases have been reported to be involved in several signal pathways which control cellular functions including cell proliferation, differentiation and death (Schlessinger et al., Neuron, 9, 383, 1992).

Accordingly, abnormal activation of protein kinases may cause diverse diseases, e.g., disorders of central nervous system, such as Alzheimer's disease (Mandelkow, E. M. et al., FEBS Lett., 314, 315, 1992; Sengupta, A. et al., Mol. Cell. Biochem., 167, 99, 1997), inflammatory disorders (Badger, J. Pharm. Exp. Ther., 279, 1453, 1996), psoriasis (Dvir et al., J. Cell Biol., 113, 857, 1991), bone disorders such as osteoporosis (Tanaka et al., Nature, 383, 528, 1996), cancers (Hunter et al., Cell, 79, 573, 1994), arteriosclerosis (Hajjar et al., FASEB J., 6, 2933, 1992), thrombosis (Salari, FEBS, 263, 104, 1990), metabolic disorders such as diabetes (Borthwick, A. C. et al., Biochem. Biophys. Res. Commun., 210, 738, 1995), vascular proliferative disorders such as angiogenesis (Strawn et al., Cancer Res., 56, 3540, 1996; Jackson et al., J. Pharm. Exp. Ther., 284, 687, 1998), stent restenosis (Buchdunger et al., Proc. Nat. Acad. Sci. USA, 92, 2258, 1991), autoimmune diseases such as transplantation rejection (Bolen et al., Ann. Rev. Immunol., 15, 371, 1997), infectious diseases such as fungus infection (International Patent Publication No. WO9805335), chronic renal failure (Liu, I. et al., Int. J. Cardiology, 69, 77-82, 1999) and chronic obstructive pulmonary disease (Nguyen, L. T. et al., Clinical Nutr., 18, 255-257, 1999; Solar, N. et al., Eur. Respir. J, 14, 1015-1022, 1997).

Aurora kinase is a Ser/Thr protein kinase involved in mitosis, and has been demonstrated to be a putative oncoprotein overexpressed in several cancer cells of breast, colon, pancreas and ovarian (Carvajal R D et al., Clin. Cancer Res., 12(23), 6869-75, 2006), and recently, there has been a report that an aurora kinase inhibitor developed by Vertex (USA) represses tumor in a nude mouse (Elizabeth A Harrington et al., Nature Medicine, 10, 262-267, 2004).

p38 mitogen-activated protein kinase (MAPK) is a proline-directed Ser/Thr kinase such as c-jun-N-terminal kinase (JNK) and extracelluar signal-regulated kinase (ERK), and it has been known to be activated by bacterial lipopolysaccharides, physico-chemical stresses, and pro-inflammatory cytokines including tumor necrosis factor (TNF-α) and interleukin-1 (IL-1), to mediate a signal pathway inducing the expression of inflammatory cytokines such as TNF-α, IL-8, IL-1 and cyclooxygenase-2.

Among such inflammatory cytokines expressed by p38 MAPK activation, TNF-α has been know to be involved in viral infections such as infection of human immunodeficiency virus (HIV), influenza virus and herpes virus, as well as inflammatory disorders such as rheumatoid inflammation, multiple sclerosis and asthma (Newton R et al., BioDrugs, 17(2), 113-129, 2003). Further, IL-8 is expressed in monocytes, fibroblasts, endothelial cells and keratinocytes to participate in inflammatory disorders, and IL-1 is expressed by activated monocytes and macrophases to take part in inflammations including rheumatoid, fever and reduction of bone resorption (Bryan Coburn et al., British Journal of Cancer, 95, 1568-1575, 2006).

c-jun-N-terminal kinase (JNK) has been demonstrated to be activated by extracellular stimuli, e.g., Fas/FasL interaction, cytokines including IL-1 and TNF-α, UV, and alteration in potassium homeostasis and osmotic pressure, to mediate a signal pathway inducing the activation of AP1 transcription factor, and participate in apoptosis and inflammatory diseases (Samadder, P. et al., J. Med. Chem., 47(10), 2710-2713, 2004).

Extracellular signal-regulated kinase (ERK) can activate other protein kinases such as Rsk90 (Bjorbaek et al., J. Biol. Chem., 270, 18848, 1995) and MAPKAP2 (Rouse et al., Cell, 78, 1027, 1994), as well as transcription factors such as ATF2 (Raingeaud et al., Mol. Cell Biol., 16, 1247, 1996), Elk-1 (Raingeaud et al., Mol. Cell Biol, 16(3), 1247-55, 1996), c-Fos (Chen et al., Proc. Natl. Acad. Sci. USA, 90, 10952, 1993) and c-Myc (Oliver et al., Proc. Soc. Exp. Biol. Med., 210, 162, 1995) to mediate the expression of several oncoprotein. Further, ERK has been reported to be overexpressed in human breast cancers (Sivaraman et al., J. Clin. Invest., 99, 1478, 1997), regulating the negative growth of breast cancer cells (Frey et al., Cancer Res., 57, 628, 1997), and it is also reported to be involved in asthma (Whelchel et al., Am. J. Respir. Cell Mol. Biol., 16, 589, 1997).

Cycline-dependent kinase (CDK) is known to play a prominent role in G1/S transition and G2/M transition in cell cycle (Kim Nasmyth, Science, 274, 1643-1677, 1996) to regulate cell growth. In particular, there have been found mutations of genes encoding CDK or CDK regulator in cancer cells in the exponential growth phage (Webster, Exp. Opin. Invest. Drugs, 7, 865-887, 1998).

Protein kinase B (PKB or AKT) is activated through the phosphatidyl inositol 3 kinase (PI3K) activation induced by Platelet derived growth factor (PDGF), nerve growth factor (NGF) or insulin-like growth factor-1 (IGF-1) (Kulik et al., Mol. Cell Biol., 17, 1595-1606, 1997; and Hemmings, B. A., Science, 275, 628-630, 1997) to mediate insulin metabolism (Calera, M. R. et al., J. Biol. Chem., 273, 7201-7204, 1998), cell differentiation and/or proliferation, as well as stress response of protein synthesis (Alessi, D. R. et al., Curr. Opin. Genet. Dev., 8, 55-62, 1998).

Further, AKT is reported to be overexpressed in several cancers (Khwaja, A., Nature, 401, 33-34, 1999; Yuan, Z. Q. et al., Oncogene, 19, 2324-2330, 2000; and Namikawa, K., et al., J. Neurosci., 20, 2875-2886, 2000), particularly in ovarian cancer (Cheng, J. Q. et al., Proc. Natl. Acad. Sci. USA, 89, 9267-9271, 1992) and pancreas cancer (Cheng, J. Q. et al., Proc. Natl. Acad. Sci. USA, 93, 3636-3641, 1996).

Glycogen synthase kinase 3 (GSK-3) known as a target protein for treating diabetes and dementia is an enzyme that phosphorylates glycogen synthase (GS) to suppress its activity. There have been reports that the activity of GSK-3 in obese diabetic mice is about twice as high as that in control (H. Eldar-Finkelman, Diabetes, 48, 1662-1666, 1999), and the activity and expression of GSK-3 in patients with type 2 diabetes is significantly higher relatively to that in normal persons (S. E. Nikoulina et al., Diabetes, 49, 263-171, 2000).

Accordingly, the present inventors have endeavored to develop a compound which is effective in inhibiting the activity of several protein kinases, and have found that an aminopyrimidine derivative can efficiently inhibit the activity of protein kinases including GSK, ERK, AKT, CDK, p38 MAPK and JNK.

### SUMMARY OF THE INVENTION

Accordingly, it is an object of the present invention to provide a novel compound that can efficiently inhibit the activity of protein kinases, and a pharmaceutically acceptable salt, hydrate, solvate and isomer thereof.

It is another object of the present invention to provide a method for preparing such compound.

It is a further object of the present invention to provide a pharmaceutical composition comprising such compound, or a pharmaceutically acceptable salt, hydrate, solvate or isomer thereof.

In accordance with one aspect of the present invention, there is provided an aminopyrimidine derivative of formula 1, and a pharmaceutically acceptable salt, hydrate, solvate and isomer thereof:

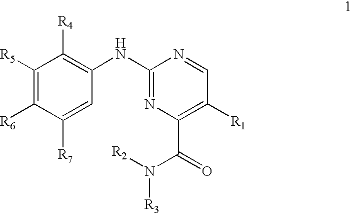

wherein,

R₁ is hydrogen, hydroxy, halogen, C₁₋₂ alkyloxy or C₁₋₂ alkyl;

R₂ is unsubstituted or substituted C₁₋₈ alkyl or C₂₋₈ alkenyl, unsubstituted or substituted C₁₋₈ alkyl or C₂₋₈ alkenyl comprising one or more nitrogen, sulfur or oxygen in its chain structure, the substituent of the alkyl or alkenyl being hydroxy; halogen; C₁₋₆ alkyloxy; C₁₋₆ alkyl; aminoalkyl; C₁₋₆ alkylamine; acetylamino; carboxyl; nitro; sulfonylamino; C₁₋₆ alkylsulfonyl; aryl optionally substituted with hydroxy, halogen, C₁₋₆ alkyloxy, C₁₋₆ alkyl, amino, C₁₋₆ alkylamino, acetylamino, carboxyl, nitro, amide, dimethyl sulfoneamino or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro or amide; aryl comprising nitrogen, sulfur or oxygen in its ring structure represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, which is unsubstituted, or substituted with hydroxy, halogen, C₁₋₆ alkyloxy, C₁₋₆ alkyl, amino, C₁₋₆ alkylamino, carbonylamino, carboxyl, nitro, C₁₋₆ trihaloalkane, sulfonylamide, C₁₋₆alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carbonyl amino, carboxyl, nitro or amide; or

unsubstituted or substituted aryl, or unsubstituted or substituted aryl comprising one or more nitrogen, sulfur or oxygen in its ring structure represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, the substituent thereof being hydroxy; halogen; C₁₋₆ alkyloxy; C₁₋₆ alkyl; amino; C₁₋₆ alkylamino; carboxyl; nitro; C₁₋₆ trihaloalkane; sulfonylamide; C₁₋₆ alkylsulfonyl; C₁₋₆ alkyl, or C₃₋₈ cycloalkyl having optional one or more nitrogen, sulfur or oxygen atoms in its chain structure as well as optional substituent selected from the group consisting of hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl and amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide;

R₃ is hydrogen; hydroxy; unsubstituted or substituted C₁₋₈ alkyl or C₃₋₈ cycloalkyl optionally having one or more nitrogen, sulfur or oxygen atoms in its chain structure, the substituent of the alkyl or cycloalkyl being hydroxyl; halogen; C₁₋₆ alkyloxy; C₁₋₆ alkyl; amino; C₁₋₆ alkylamino; carboxyl; nitro; sulfonylamide; C₁₋₆ alkylsulfonyl; amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide; or

R₂ and R₃ are fused together with the nitrogen to which they are attached to form a morpholine ring, and

R₄, R₅, R₆ and R₇ are each independently hydrogen, hydroxy, halogen, amine substituted with C₁₋₆ alkyl or C₃₋₆ cycloalkyl having optional substituent, or amine substituted with C₁₋₈ alkyl or C₃₋₆ cycloalkyl comprising one or more nitrogen, sulfur or oxygen in its chain structure, the substituent of the alkyl or cycloalkyl being hydroxyl; halogen; C₁₋₆ alkyloxy; C₁₋₆ alkyl; amino; C₁₋₆ alkylamino; carboxyl; nitro; sulfonylamide; C₁₋₆ alkylsulfonyl; amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide; or

R₆ is fused together with R₅ or R₇ to form a dioxorane ring.

### DETAILED DESCRIPTION OF THE INVENTION

Among the compound of the formula 1, preferred are those wherein R₁ is H, Cl or Br; R₂ is H, or unsubstituted or substituted C₁₋₆ alkyl; R₃ is H, or unsubstituted or substituted C₁₋₆ alkyl; R₄ is aminoC₁₋₆ alkylamine substituted with halogen (e.g., F or Cl), C₁₋₆ alkoxy or substituted C₁₋₆ alkyl; R₅ is amino C₁₋₆ alkylamine substituted with halogen (e.g., F or Cl), C₁₋₆ alkoxy, substituted C₁₋₆ alkyl or substituted ring compound; R₆ is amino C₁₋₆ alkylamine substituted with halogen (e.g., F or Cl), C₁₋₆ alkoxy or substituted C₁₋₆ alkyl; R₇ is aminoC₁₋₆ alkylamine substituted with halogen (e.g., F or Cl), C₁₋₆ alkoxy or substituted C₁₋₆ alkyl.

Representative examples of the inventive compound are shown in Table 1.

TABLE 1

| Ex- | | | |
|---|---|---|---|
| am- | | | |
| mple | Structure | Nomenclature | NMR |
| 1 | 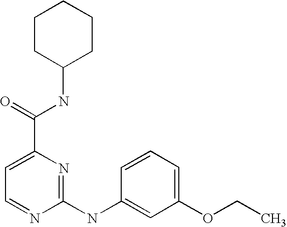 | 2-(3-ethoxy- phenylamino)- pyrimidine-4- carboxylic acid cyclohexylamide | ¹H NMR (CDCl₃, 300 MHz) δ 0.83-0.90(m, 3 H), 1.33-1.48 (m, 6 H), 1.60-1.66(m, 2 H), 3.08-3.19(m, 3 H), 3.29-3.34 (m, 2 H), 3.87(s, 3 H), 3.97(s, 3 H), 6.97(dd, 1 H), 7.73(dd, 1 H), 8.21-8.23(m, 1 H), 8.41(s, 1 H), 8.84(s, 1 H) |
| 2 | 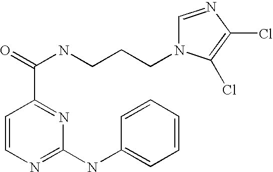 | 2-phenylamino- pyrimidine-4- carboxylic acid[3- (4,5-dichloro- imidazole-1-yl)- propyl]-amide | ¹H NMR (CD₃OD, 300 MHz) δ 2.83(m, 2 H), 3.35(m, 2 H), 3.83(m, 2 H), 6.61-7.52(m, 5 H), 7.04(s, 1 H), 7.61(d, 1 H), 8.83(d, 1 H). |
| 3 | 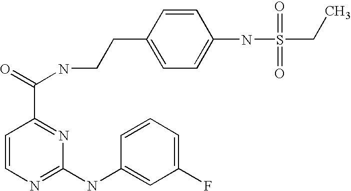 | 2-(3-fluoro- phenylamino)- pyrimidine-4- carboxylic acid[2-(4- ethylsulfonylamino- phenyl)-ethyl]-amide | ¹H NMR (CD₃OD, 300 MHz) δ 1.23(t, 3 H), 2.80(t, 2 H), 3.25(t, 2 H), 3.44(q, 2 H), 6.86-7.63(m, 4 H), 7.04(d, 2 H), 7.44(d, 2 H), 7.62(d, 1 H), 8.83(d, 1 H). |
| 4 | 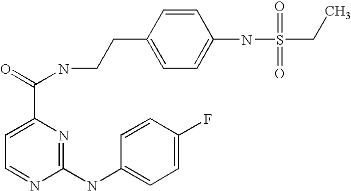 | 2-(4-fluoro- phenylamino)- pyrimidine-4- carboxylic acid[2-(4- ethylsulfonylamino- phenyl)-ethyl]-amide | ¹H NMR (CD₃OD, 300 MHz) δ 1.23(t, 3 H), 2.80(t, 2 H), 3.25(t, 2 H), 3.44(q, 2 H), 6.66(d, 2 H), 7.04(d, 2 H), 7.44(d, 2 H), 7.52(d, 2 H), 7.63(d, 1 H), 8.77(d, 1 H). |
| 5 | 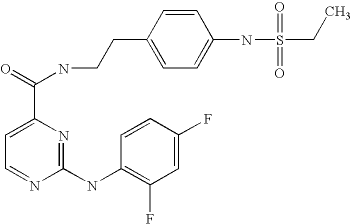 | 2-(2,4-difluoro- phenylamino)- pyrimidine-4- carboxylic acid[2-(4- ethylsulfonylamino- phenyl)-ethyl]-amide | ¹H NMR (CD₃OD, 300 MHz) δ 1.27(t, 3 H), 2.83(t, 2 H), 3.22(t, 2 H), 3.49(q, 2 H), 6.56-7.61(m, 3 H), 7.01(d, 2 H), 7.46(d, 2 H), 7.61(d, 1 H), 8.80(d, 1 H). |
| 6 | 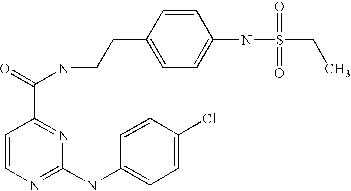 | 2-(4-chloro- phenylamino)- pyrimidine-4- carboxyli acid[2-(4- ethylsulfonylamino- phenyl)-ethyl]-amide | ¹H NMR (CD₃OD, 300 MHz) δ 1.25(t, 3 H), 2.83(t, 2 H), 3.21(t, 2 H), 3.49(q, 2 H), 6.56(d, 2 H), 7.01(d, 2 H), 7.44(d, 2 H), 7.51(d, 2 H), 7.68(d, 1 H), 8.79(d, 1 H). |
| 7 | 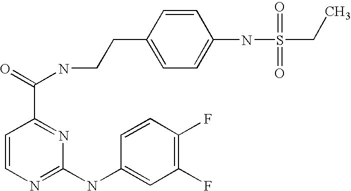 | 2-(3,4-difluoro- phenylamino)- pyrimidine-4- carboxylic acid[2-(4- ethylsulfonylamino- phenyl)-ethyl]-amide | ¹H NMR (CD₃OD, 300 MHz) δ 1.26(t, 3 H), 2.73(t, 2 H), 3.29(t, 2 H), 3.48(q, 2 H), 6.46-7.54(m, 3 H), 7.02(d, 2 H), 7.46(d, 2 H), 7.63(d, 1 H), 8.83(d, 1 H). |
| 8 | 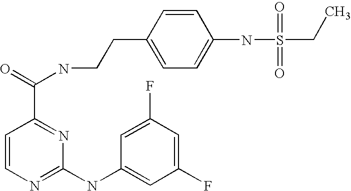 | 2-(3,5-difluoro-phenylamino)- pyrimidine-4-carboxylic acid[2-(4-ethylsulfonylamino- phenyl)-ethyl]-amide | ¹H NMR (CD₃OD, 300 MHz) δ 1.27(t, 3 H), 2.83(t, 2 H), 3.22(t, 2 H), 3.49(q, 2 H), 6.56-7.61(m, 3 H), 7.01(d, 2 H), 7.46(d, 2 H), 7.61(d, 1 H), 8.80(d, 1 H). |
| 9 | 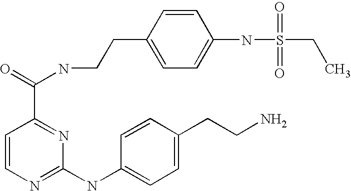 | 2-[4-(2-amino-ethyl)- phenylamino]-pyrimidine-4- carboxylic acid[2-(4- ethylsulfonylamino-phenyl)- ethyl]-amide | ¹H NMR (CD₃OD, 300 MHz) δ 1.25(t, 3 H), 2.81(t, 2 H), 2.83(t, 2 H), 2.93(t, 2 H), 3.21(t, 2 H), 3.41(q, 2 H), 6.64(d, 2 H), 7.03(d, 2 H), 7.54(d, 2 H), 7.58(d, 2 H), 7.63(d, 1 H), 8.77(d, 1 H). |
| 10 | 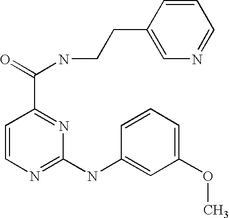 | 2-(3-methoxy-phenylamino)- pyrimdine-4-carboxylic acid(2-pyridine-3-yl-ethyl)- amide | ¹H NMR (CD₃OD, 300 MHz) δ 3.21(t, 2 H), 3.80(t, 2 H), 6.56(m, 1 H), 7.40~7.35(m, 3 H), 8.00(m, 1 H), 8.71(d, 1 H), 8.74(m, 2 H), 8.83(s, 1 H). |
| 11 | 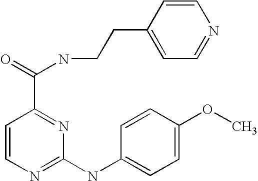 | 2-(4-methoxy-phenylamino)- pyrimidine-4-carboxylic acid(2-pyridine-4-yl-ethyl)- amide | ¹H NMR (CD₃OD, 300 MHz) δ 3.21(t, 2 H), 3.80(t ,2 H), 6.56(m, 1 H), 7.40~7.35(m, 3 H), 8.00(m, 1 H), 8.71(d, 1 H), 8.74(m, 2 H), 8.83(s, 1 H). |
| 12 | 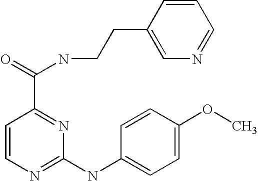 | 2-(4-methoxy-phenylamino)- pyrimidine-4-carbooxylic acid(2-pyridine-3-yl-ethyl)- amide | ¹H NMR (CD₃OD, 300 MHz) δ 3.15(t, 2 H), 3.75-3.92(m, 5 H), 7.22(d, 1 H), 8.00(m, 1 H), 8.52(m, 1 H), 8.67-8.80(m, 2 H), 9.05(s, 1 H). |
| 13 | 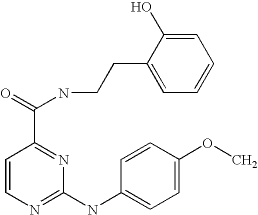 | 2-(4-methoxy-phenylamino)- pyrimidine-4-carboxylic acid[2-(2-hydroxy-phenyl)- ethyl]-amide | ¹H NMR (CD₃OD, 300 MHz) δ 2.91(t, 2 H), 3.64(t, 2 H), 3.79(s, 3 H), 6.72-6.80(m, 2 H), 6.87(d, 2 H), 7.00-7.18(m, 2 H), 7.28(m, 1 H), 7.09(d, 2 H), 8.57(d, 1 H). |
| 14 | 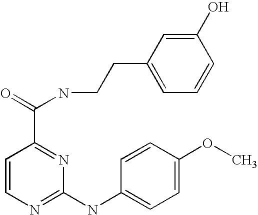 | 2-(4-methoxy-phenylamino)- pyrimidine-4-carboxylic acid[2-(3-hydroxy-phenyl)- ethyl]-amide | ¹H NMR (CD₃OD, 300 MHz) δ 2.84(t, 2 H), 3.62(t, 2 H), 3.79(s, 3 H), 6.67-6.72(m, 4 H), 6.73(d, 2 H), 7.10(t, 1 H), 7.34 m, 1 H), 7.09(d, 2 H), 8.57(d, 1 H). |
| 15 | 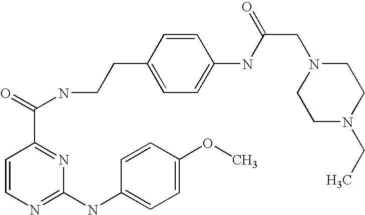 | 2-(4-methoxy- phenylamino)-pyrimidine- 4-carboxylic acid(2-{4-[2- (4-ethyl-piperazin-1-yl)- acetylamino]-phenyl}- ethyl)-amide | ¹H NMR (CD₃OD, 300 MHz) δ 1.02(t, 3 H), 2.35(d, 4 H), 2.29(q, 2 H), 2.38(t, 2 H), 3.34(s, 2 H), 3.35(s, 3 H), 3.59(t, 2 H), 6.73(d, 2 H), 7.03(d, 2 H), 7.34(d, 2 H), 7.59(d, 2 H), 7.67(d, 1 H), 8.57(d, 1 H) |
| 16 | 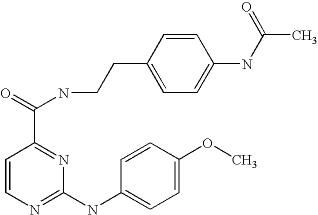 | 2-(4-methoxy- phenylamino)-pyrimidine- 4-carboxylic acid[2-(4- acetylamino-phenyl)-ethyl]- amide | ¹H NMR (CD₃OD, 300 MHz) δ 2.35(d, 2 H), 2.39(s, 2 H), 3.34(s, 2 H), 3.35(s, 3 H), 6.63(d, 2 H), 7.09(d, 2 H), 7.44(d, 2 H), 7.55(d, 2 H), 7.57(d, 1 H), 8.66(d, 1 H) |
| 17 | 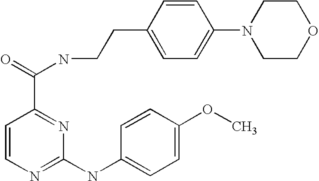 | 2-(4-methoxy- phenylamino)-pyrimidine- 4-carboxylic acid[2-(4- morpholine-4-yl-phenyl)- ethyl]-amide | ¹H NMR (CD₃OD, 300 MHz) δ 2.33(d, 4 H), 2.31(t, 2 H), 3.35(s, 2 H), 3.38(s, 3 H), 6.53(d, 2 H), 7.01(d, 2 H), 7.54(d, 2 H), 7.59(d, 2 H), 7.62(d, 1 H), 8.55(d, 1 H) |
| 18 | 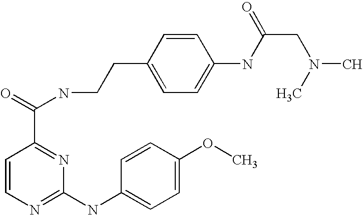 | 2-(4-methoxy- phenylamino)-pyrimidine- 4-carboxylic acid{2-[4-(2- dimethylamino- acetylamino)-phenyl]- ethyl}-amide | ¹H NMR (CD₃OD, 300 MHz) δ 2.38(t, 2 H), 3.34(s, 2 H), 3.35(s, 3 H), 3.49(s, 6 H), 3.59(t, 2 H), 6.53(d, 2 H), 7.01(d, 2 H), 7.32(d, 2 H), 7.51(d, 2 H), 7.63(d, 1 H), 8.55(d, 1 H) |
| 19 | 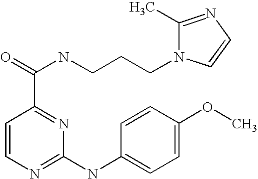 | 2-(4-methoxy- phenylamino)-pyrimidine- 4-carboxylic acid[3-(2- methyl-imidazole-1-yl)- propyl]-amide | ¹H NMR (CD₃OD, 300 MHz) δ 2.51(s, 3 H), 2.58(m, 2 H), 3.18(t, 2 H), 3.59(s, 3 H), 4.04(t, 2 H), 6.74-6.96(m, 2 H), 7.02(d, 2 H), 7.78(d, 2 H), 7.63(d, 1 H), 8.55(d, 1 H) |
| 20 | 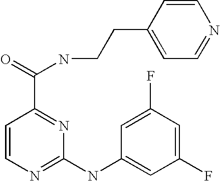 | 2-(3,5-difluoro- phenylamino)-N-(2- (pyridine-4- yl)ethyl) pyrimidine-4- carboxyamide | ¹H NMR (CD₃OD, 300 MHz) δ 3.25(t, 2 H), 3.84(t, 2 H), 6.55(m, 1 H), 7.35~7.39(m, 3 H), 8.00(br, 2 H), 8.71(m, 3 H). |
| 21 | 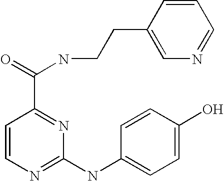 | 2-(4-hydroxy- phenylamino)-N-(2- (pyridin-2- yl)ethyl)pyrimidine-4- carboxyamide | ¹H NMR (CD₃OD, 300 MHz) δ 2.58(m, 2 H), 3.18(t, 2 H), 4.04(t, 2 H), 6.74-7.14(m, 4 h), 7.02(d, 2 H), 7.78(d, 2 H), 7.63(d, 1 H), 8.55(d, 1 H) |
| 22 | 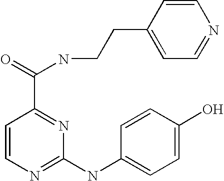 | 2-(4-hydroxy-phenylamino)- pyrimidine-4-carboxylic acid(2- pyridine-3-yl-ethyl)-amide | ¹H NMR (CD₃OD, 300 MHz) δ 2.58(m, 2 H), 3.18(t, 2 H), 4.04(t, 2 H), 6.79-7.34(m, 4 h), 7.01(d, 2 H), 7.58(d, 2 H), 7.67(d, 1 H), 8.65(d, 1 H) |
| 23 | 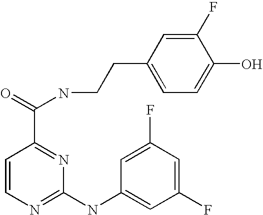 | 2-(3,5-difluoro-phenylamino)- N- (3-fluoro-4-hydroxy)pyrimidine-4- carboxyamide | ¹H NMR (CD₃OD, 300 MHz) δ 2.53(m, 2 H), 3.28(t, 2 H), 4.01(t, 2 H), 6.49-7.14(m, 3 H), 7.01- 7.49(m, 3 H), 7.67(d, 1 H), 8.65(d, 1 H) |
| 24 | 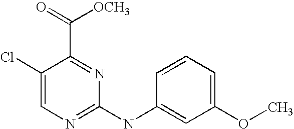 | methyl-5-chloro-2-(3- fluorophenylamino)pyrimidine- 4- carboxylate | ¹H NMR (CD₃OD, 300 MHz) δ 3.83(s, 3 H), 3.88(s, 3 H), 6.18-7.19(m, 4 H), 8.37(s, 1 H) |
| 25 | 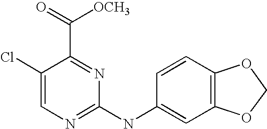 | 2-(benzo[1,3]dioxol-5- ylamino)-5- chloro-pyrimidine-4-carobxylic acid(3-phenyl-propyl)-amide | ¹H NMR (CD₃OD, 300 MHz) δ 3.83(s, 3 H), 4.01(s, 2 H), 6.28-7.39(m, 3 H), 8.33(s, 1 H) |
| 26 | 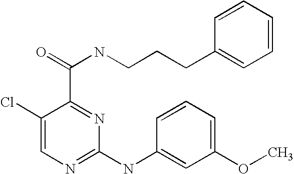 | 5-chloro-2-phenylamino- pyrimidine-4-carboxylic acid(3- phenyl-propyl)-amide | H NMR (CD₃OD, 300 MHz) δ 2.03(m, 2 H), 2.63(t, 2 H), 3.18(t, 2 H), 3.83(s, 3 H), 6.18-7.29(m, 4 H), 7.25(d, 2 H), 7.43- 7.55(m, 3 H), 8.33(s, 1 H). |
| 27 | 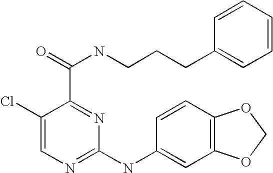 | 2-(benzole[1,3]dioxol-5- ylamino)- 5-chloro-pyrimi- dine-4-carboxylic acid[3-(4,5-dichloro-imi- dazole-1- yl)-propyl]-amide | ¹H NMR (CD₃OD, 300 MHz) δ 2.03(m, 2 H), 2.63(t, 2 H), 3.18(t, 2 H), 4.02(s, 2 H), 6.11-7.31(m, 3 H), 7.24(d, 2 H), 7.41- 7.65(m, 3 H), 8.42(s, 1 H). |
| 28 | 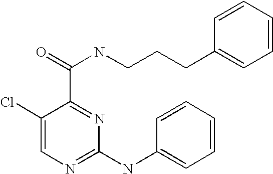 | 5-chloro-2-(3-methoxy- phenylamino)-pyrimidine-4- carboxylic acid[3-(4,5-dichloro- imidazole-1-yl)-propyl]-amide | ¹H NMR (CD₃OD, 300 MHz) δ 1.43-1.69(m, 2 H), 2.58(d, 2 H), 4.03(d, 2 H), 6.01-7.11(m, 5 H), 7.14(s, 1 H), 8.60(s, 1 H). |
| 29 | 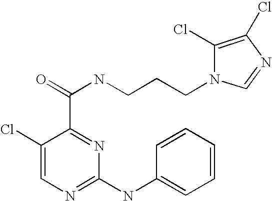 | 5-chloroN-(3-(4,5- dichloro-1H-imidazolyl-1- yl)propyl)-2- phenylamino)pyrimidine- 4-carboxyamide | ¹H NMR (CD₃OD, 300 MHz) δ 1.43-1.69(m, 2 H), 2.58(d, 2 H), 4.03(d, 2 H), 6.01-7.11(m, 5 H), 7.14(s, 1 H), 8.60(s, 1 H). |
| 30 | 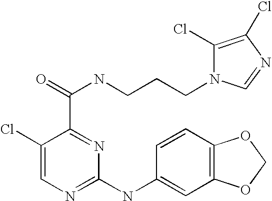 | 2-(benzo[d][1,3]dioxalyl- 5-amino)-5-chloro-N- methylpyrimidine-4- carboxyamide | ¹H NMR (CD₃OD, 300 MHz) δ 1.45-1.71(m, 2 H), 2.58(d, 2 H), 3.18(d, 2 H), 4.03(m, 2 H), 6.11-7.23 (m, 4 H), 7.15(s, 1 H), 8.63(s, 1 H). |
| 31 | 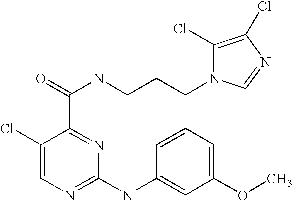 | 5-chloroN-(3-(4,5- dichloro-1H-imidazolyl-1- yl)propyl)-2-(3- methoxyphenylamino)pyri- midine-4-carboxyamide | ¹H NMR (CD₃OD, 300 MHz) δ 2.58(d, 2 H), 3.18(d, 2 H), 3.83(s, 3 H), 4.04(d, 2 H), 6.18-7.19(m, 4 H), 7.15(s, 1 H), 8.61(s, 1 H). |
| 32 | 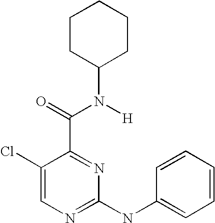 | 5-chloro-2-phenylamino- pyrimidine-4-carboxylic acid cyclohexylamide | ¹H NMR (CD₃OD, 300 MHz) δ 1.32-1.41(m, 4 H), 1.46-1.68(m, 2 H), 1.54-1.83(m, 2 H), 1.91- 1.95(m, 2 H), 3.72-3.93(m, 1 H), 6.11- 7.15(m, 5 H), 6.15(s, 1 H), 8.69(s, 1 H). |
| 33 | 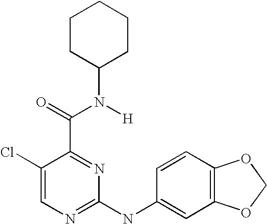 | 2-(benzo[1,3]dioxol-5- ylamino)-5-chloro- pyrimidine-4-carboxylic acid cyclohexylamide | ¹H NMR (CD₃OD, 300 MHz) δ 1.31-1.43(m, 4 H), 1.45-1.71(m, 2 H), 1.56-1.89(m, 2 H), 1.90- 1.93(m, 2 H), 3.72- 3.93(m, 1 H), 3.81(s, 2 H), 6.68(d, 1 H), 8.61(m, 1 H). |
| 34 | 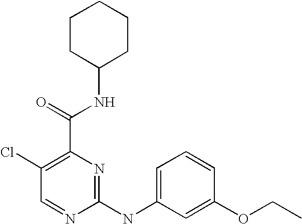 | 5-chloro-2-(3-ethoxy- phenylamino)-pyrimidine- 4-carboxylic acidcyclohexylamide; | ¹H NMR (CD₃OD, 300 MHz) δ 1.33-1.44(m, 4 H), 1.23(t, 3 H), 1.48-1.66(m, 2 H), 1.56-1.86(m, 2 H), 1.94-1.97(m, 2 H), 3.72- 3.93(m, 1 H), 4.04(q, 2 H), 6.18- 7.19(m, 4 H), 6.18- 7.19(m, 4 H), 4.10(q, 2 H), 7.63(d, 1 H). |
| 35 | 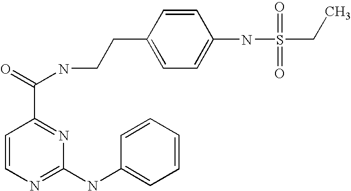 | 2-phenylamino- pyrimidine-4- carboxylic acid[2-(4- ethylsulfonylamino- phenyl)-ethyl]-amide | ¹H NMR (CD₃OD, 300 MHz) δ 1.27(t, 3 H), 2.83(t, 2 H), 3.37(t, 2 H), 3.45(q, 2 H), 6.81-7.63(m, 5 H), 7.04(d, 2 H), 7.44(d, 2 H), 7.63(d, 1 H), 8.84(d, 1 H). |
| 36 | 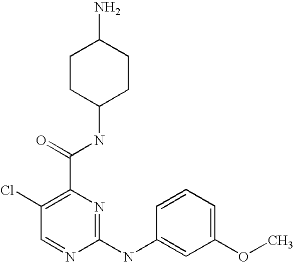 | 5-chloro-2-(3-methoxy- phenylamino)- pyrimidine-4- carboxylic acid(4- amino-cyclohexyl)- amide | ¹H NMR (CD₃OD, 300 MHz) δ 1 1.33-1.44(m, 4 H), 1.48-1.66(m, 2 H), 1.56-1.86(m, 2 H), 1.94- 1.97(m, 2 H), 3.76(s, 3 H), 6.60-7.58(m, 4 H), 8.64(d, 1 H). |
| 37 | 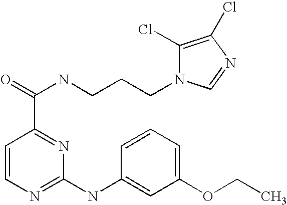 | 2-(3-ethoxy- phenylamino)- pyrimidine-4- carboxylic acid[3-(4,5- dichloro-imidazole-1- yl)-propyl]-amide | ¹H NMR (CD₃OD, 300 MHz) δ 2.58(d, 2 H), 2.38(t, 3 H), 3.18(d, 2 H), 3.82(q, 2 H), 4.04(d, 2 H), 6.18-7.19 (m, 4 H), 7.15(s, 1 H), 7.23(s, 1 H), 8.61(s, 1 H). |
| 38 | 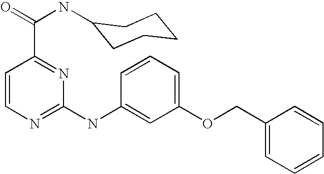 | 2-(3-benzyloxy- phenylamino)- pyrimidine-4- carboxylic acid cyclohexylamide | ¹H NMR (CD₃OD, 300 MHz) δ 1.31 (d, 3 H), 1.33-1.44(m, 4 H), 1.48-1.66 (m, 2 H), 1.56-1.86(m, 2 H), 1.94- 1.97(m, 2 H), 3.76- 3.96(m, 1 H), 4.10(q, 2 H), 6.60(d, 1 H), 7.10(d, 1 H), 7.34(d, 2 H), 7.41(s, 1 H), 8.64(d, 1 H) |
| 39 | 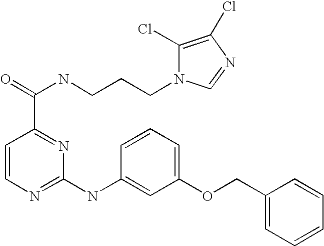 | 2-(3-benzyloxy- phenylamino)- pyrimidine-4- carboxylic acid[3-(4,5- dichloro-imidazole-1- yl)-propyl]-amide; | ¹H NMR (CD₃OD, 300 MHz) δ 2.48(d, 2 H), 2.36(t, 3 H), 3.28(d, 2 H), 3.81(q, 2 H), 4.14(d, 2 H), 5.66(s, 2 H), 6.11-7.87(m, 9 H), 7.15(s, 1 H), 7.53(s, 1 H), 8.62(s, 1 H). |
| 40 | 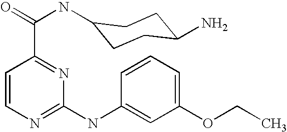 | 2-(3-ethoxy- phenylamino)- pyrimidine-4- carboxylic acid(4- aminocyclohexyl)- amide | ¹H NMR (CDCl₃, 300 MHz) δ 1.39 (t, 2 H), 1.42-1.53(m, 4 H), 1.78-2.01 (m, 3 H), 2.33-2.45(m, 2 H), 3.20- 3.28(m, 1 H), 3.89-4.00(m, 1 H), 4.04(q, 2 H), 6.58-6.62(m, 1 H), 7.14-7.22(m, 2 H), 7.33-7.34(m, 1 H), 7.37-7.39(m, 1 H), 8.63-8.65 (m, 1 H). |
| 41 | 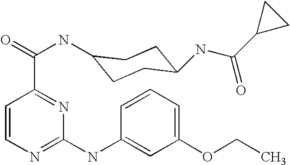 | 2-(3-ethoxy- phenylamino)- pyrimidine-4- carboxylic acid[4- (cyclopropanecarbonyl- amino)-cyclohexyl]- amide | ¹H NMR (CD₃OD, 300 MHz) δ 1 1.33-1.44(m, 8 H), 1.48-1.66(m, 3 H), 1.56-1.98(m, 4 H), 2.87(q, 2 H), 3.89(s, 3 H), 6.61-7.68(m, 4 H), 7.33(d, 1 H), 8.67(d, 1 H). |
| 42 | 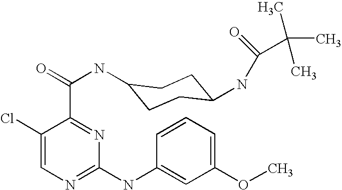 | 5-chloro-2-(3-methoxy- phenylamino)- pyrimidine-4- carobxylic acid[4-(2,2- dimethyl- propionylamino)- cyclohexyl]-amide | ¹H NMR (CD₃OD, 300 MHz) δ 1 1.33-1.44(m, 8 H), 1.48-1.66(m, 3 H), 1.56-1.98(m, 4 H), 1.99(s, 9 H), 2.87(q, 2 H), 3.89(s, 3 H), 6.61- 7.68(m, 4 H), 7.35(d, 1 H), 8.63(d, 1 H). |
| 43 | 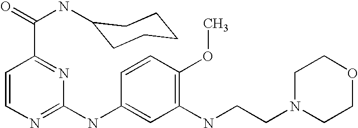 | 2-[4-methoxy-3-(2- morpholine-4-yl- ethylamino)- phenylamino]- pyrimidine-4- carboxylic acid cycloehxylamide | ¹H NMR (CD₃OD, 300 MHz) δ 1.11-1.79(m, 10 H), 2.36(s, 4 H), 2.50(t, 2 H), 3.45(t, 2 H), 3.54(m, 1 H), 3.65(s, 4 H), 3.84(s, 3 H), 5.63-6.42(m, 3 H), 7.63(d, 1 H), 8.84(s, 1 H). |
| 44 | 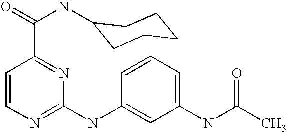 | 2-(3-acetylamino- phenylamino)- pyrimidine-4- carboxylic acid cyclohexylamide | ¹H NMR (CD₃OD, 300 MHz) δ 1.11-1.74(m, 10 H), 2.04(s, 3 H), 3.54(m, 1 H), 6.59- 7.37(m, 4 H), 7.63(s, 1 H), 8.84(d, 1 H). |
| 45 | 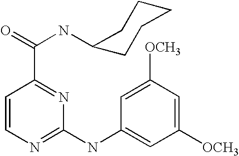 | 2-(3,5-dimethoxy- phenylamino)- pyrimidine-4- carboxylic acid cyclohexylamide | ¹H NMR (CD₃OD, 300 MHz) δ 1.11-1.74(m, 10 H), 3.83(s, 6 H), 3.54(m, 1 H), 5.74- 5.73(m, 3 H), 7.63(s, 1 H), 8.84(d, 1 H). |
| 46 | 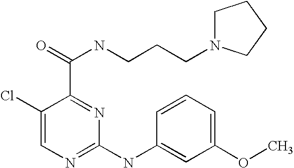 | 5-chloro-2-(3-methoxy- phenylamino)- pyrimidine-4- carboxylic acid(3- pyrrolindin-1-yl- propyl)-amide | ¹H NMR (CD₃OD, 300 MHz) δ 1.68(s, 4 H), 1.70(m, 2 H), 2.41(t, 2 H), 2.51(s, 4 H), 3.18(t, 2 H), 3.83(s, 3 H), 6.18-7.19(m, 3 H), 8.42(s, 1 H). |
| 47 | 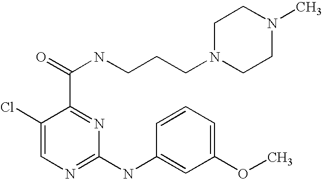 | 5-chloro-2-(3-methoxy- phenylamino)- pyrimidine-4- carboxylic acid[3-(4- methyl-piperazin-1-yl)- propyl]-amide | ¹H NMR (CD₃OD, 300 MHz) δ 1.70(m, 2 H), 2.26(s, 3 H), 2.35(s, 8 H), 3.83(s, 3 H), 6.18-7.19(m, 3 H), 8.42(s, 1 H). |
| 48 | 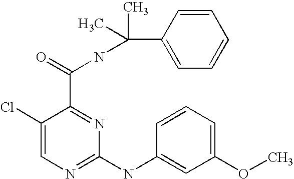 | 5-chloro-2-(3-methoxy- phenylamino)-pyrimidine-4- carboxylic acid(1-methyl-1- phenyl-ethyl)-amide | ¹H NMR (CD₃OD, 300 MHz) δ 1.47(s, 6 H), 3.83(s, 3 H), 6.18-7.19(m, 3 H), 7.30- 7.37(m, 5 H), 8.42(s, 1 H). |
| 49 | 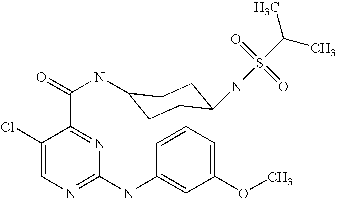 | 5-chloro-2-(3-methoxy- phenylamino)-pyrimidine-4- carboxylic acid[4-(propane-2- sulfonylamino)-cyclohexyl]- amide | ¹H NMR (CD₃OD, 300 MHz) δ 1.33(s, 6 H), 3.34(m, 1 H), 1.74-3.54(m, 10 H), 3.83(s, 3 H), 6.18-871.9(m, 4 H), 8.42(s, 1 H). |
| 50 | 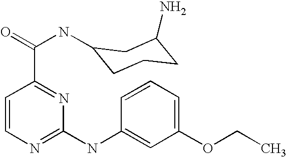 | 2-(3-ethoxy-phenylamino)- pyrimdine-4-carboxylic acid(3-aminocyclohexyl)- amide | ¹H NMR (CD₃OD, 300 MHz) δ 1.29-3.57(m, 10 H), 1.32(t, 3 H), 4.09(q, 2 H), 6.18- 87.19(m, 4 H), 7.63(s, 1 H), 8.84(s, 1 H) |
| 51 | 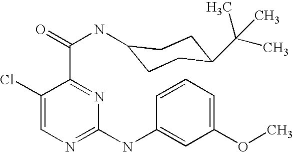 | 5-chloro-2-(3-methoxy- phenylamino)-pyrimidine-4- carboxylic acid(4-tert-butyl- cyclohexyl)-amide | ¹H NMR (CD₃OD, 300 MHz) δ 0.94(s, 9 H), 1.49-3.54(m, 10 H), 3.83(s, 3 H), 6.18- 7.19(m, 4 H), 8.42(s, 1 H) |
| 52 | 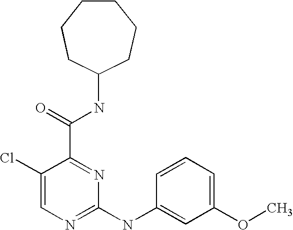 | 5-chloro-2-(3-methoxy- phenylmaino)-pyrimidine-4- carboxylic acid cycloheptylamide | ¹H NMR (CD₃OD, 300 MHz) δ 1.23-1.24(m, 2 H), 1.29-1.80 (m, 8 H), 1.99-2.01(m, 2 H), 3.80(s, 3 H), 3.99-4.08(m, 1 H), 6.59-6.60(m, 1 H), 7.18- 7.21(m, 2 H), 7.41-7.43(m, 1 H), 8.49(s, 1 H). |
| 53 | 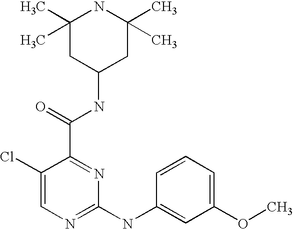 | 5-chloro-2-(3-methoxy- phenylamino)-pyrimidine-4- carboxylic acid(2,2,6,6- tetramethyl-piperidine-4-yl)- amide | ¹H NMR (CD₃OD, 300 MHz) δ 1.32(s, 12 H), 1.52-3.60(m, 5 H), 3.83(s, 3 H), 6.18-7.19(m, 4 H), 8.42(s, 1 H) |
| 54 | 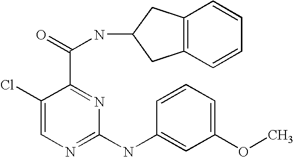 | 5-chloro-2-(3-methoxy- phenylamino)-pyrimidine-4- carboxylic acid inden-2-yl amide; | ¹H NMR (CD₃OD, 300 MHz) δ 2.88-4.13(m, 5 H), 3.83(s, 3 H), 6.18-7.19(m, 4 H), 7.20(s, 4 H), 8.42(s, 1 H). |
| 55 | 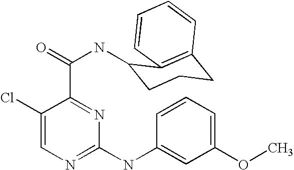 | 5-chloro-2-(3-methoxy- phenylamino)-pyrimidine-4- carboxylic acid(1,2,3,4- tetrahydro-naphthalen-1-yl)- amide | ¹H NMR (CD₃OD, 300 MHz) δ 1.95-4.95(m, 7 H), 3.83(s, 3 H), 6.92(m, 4 H), 6.18-7.19(m, 4 H), 8.42(s, 1 H). |
| 56 | 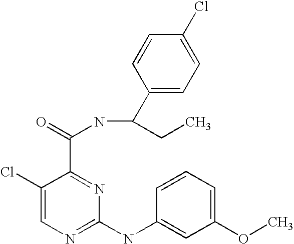 | 5-chloro-2-(3-methoxy- phenylamino)-pyrimidine-4- carboxylic acid[1-(4-chloro- phenyl)-propyl]-amide | ¹H NMR (CD₃OD, 300 MHz) δ 0.90(q, 3 H), 1.88(t, 2 H), 3.83(s, 3 H), 4.87(m, 1 H), 6.18-7.19(m, 4 H), 7.44-7.48(m, 4 H), 8.42(s, 1 H). |
| 57 | 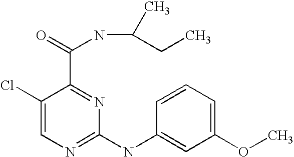 | 5-chloro-2-(3-methoxy- phenylamino)-pyrimidine-4- carboxylic acid sec-butylamide | ¹H NMR (CD₃OD, 300 MHz) δ 1.00(t, 3 H), 1.22(d, 3 H), 1.55- 1.61(m, 2 H), 3.78(s, 3 H), 3.98- 4.00(m, 1 H), 6.58-6.61(m, 1 H), 7.15-7.18(m ,2 H), 7.41- 7.42(m, 1 H), 8.49(s, 1 H). |
| 58 | 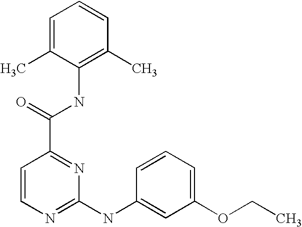 | 2-(3-ethoxy-phenylamino)- pyrimidine-4-carboxylic acid(2,6-dimehtyl-phenyl)- amide | ¹H NMR (CD₃OD, 300 MHz) δ 1.32(q, 3 H), 2.12(s, 6 H), 4.09(q, 2 H), 4.87(m, 1 H), 6.18-7.19(m, 4 H), 7.44-7.48(m, 7 H), 7.63(s, 1 H), 8.84(s, 1 H). |
| 59 | 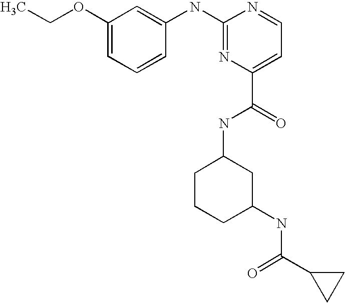 | 2-(3-ethoxy-phenylamino)- pyrimidine-4-carboxylic acid[3-(cyclopropane carbonyl-amino)-cyclohexyl]- amide | ¹H NMR (CD₃OD, 300 MHz) δ 0.64-0.89(m, 4 H), 1.16(m, 1 H), 1.32(t, 3 H), 1.49-3.54(M, 10 H), 4.09(t, 2 H), 6.18-7.19(m, 4 H), 7.63(d, 1 H), 8.83(d, 1 H). |
| 60 | 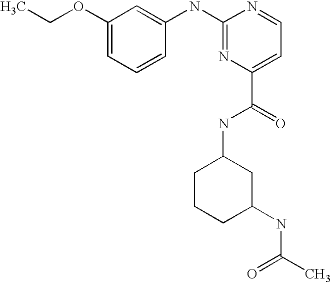 | 2-(3-ethoxy-phenylamino)- pyrimidine-4-carboxylic acid(3-acetylamino- cyclohexyl)-amide | ¹H NMR (CD₃OD, 300 MHz) δ 1.84(s, 3 H), 1.32(t, 3 H), 1.49-3.54(m, 10 H), 4.09(t, 2 H), 6.21-7.17(m, 4 H), 7.53(d, 1 H), 8.77(d, 1 H). |
| 61 | 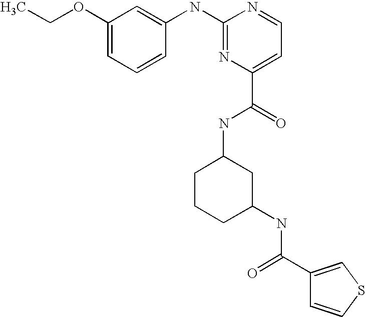 | 2-(3-ethoxy-phenylamino)- pyrimidine-4-carboxylic acid{3-[(thiophene-3- carbonyl)-amino]- cyclohexyl}-amide | ¹H NMR (CD₃OD, 300 MHz) δ 1.32(t, 3 H), 1.49-3.54(m, 10 H), 4.04(t, 2 H), 6.17-7.19(m, 4 H), 7.27(m, 1 H), 7.63(d, 1 H), 8.14-8.30(m, 2 H), 8.82(d, 1 H). |
| 62 | 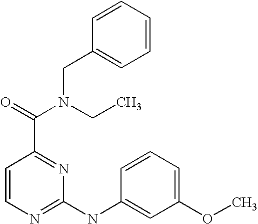 | 2-(3-methoxy-phenylamino)- pyrimidine-4-carboxylic acid benzyl-ethyl-amide | ¹H NMR (CD₃OD, 300 MHz) δ 0.98(t, 3 H), 1.88-4.87(m, 3 H), 6.17-7.19(m, 4 H), 7.29- 7.40(m, 5 H), 7.63(d, 1 H), 8.83(d, 1 H). |
| 63 | 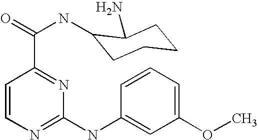 | 2-(3-methoxy-phenylamino)- pyrimidine-4-carboxylic acid(2- amino-cyclohexyl)-amide | ¹H NMR (CD₃OD, 300 MHz) δ 1.21-3.77(m, 10 H), 3.38(s, 3 H), 6.12-7.23(m, 4 H), 7.63(d, 1 H), 8.82(d, 1 H). |
| 64 | 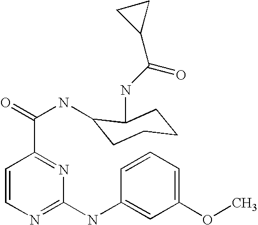 | 2-(3-ethoxy-phenylamino)- pyrimidine-4-carboxylic acid[2- (cyclopropanecarbonyl-amino)- cyclohexyl]-amide | ¹H NMR (CD₃OD, 300 MHz) δ 0.64-0.86(m, 4 H), 1.16(m, 1 H), 1.11-4.16(m, 10 H), 3.83(s, 3 H), 6.11-7.09(m, 4 H), 7.63(d, 1 H), 8.84(d, 1 H). |
| 65 | 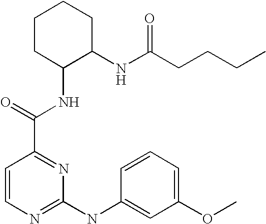 | 2-(3-methoxy-phenylamino)- pyrimidine-4-carboxylic acid(2- butylamino-cyclohexyl)-amide | ¹H NMR (CD₃OD, 300 MHz) δ 0.90-2.05(m, 8 H), 1.11-4.11(m, 10 H), 3.80(s, 3 H), 6.18-7.19(m, 4 H), 7.61(d, 1 H), 8.79(d, 1 H). |
| 66 | 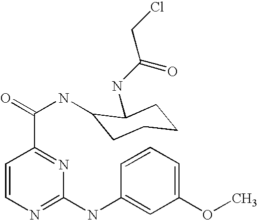 | 2-(3-methoxy-phenylamino)- pyrimidine-4-carboxylic acid[2- (2-chloro-acetylamino)- cyclohexyl]-amide | ¹H NMR (CD₃OD, 300 MHz) δ 0.90-2.05(m, 6 H), 1.11-4.11(m, 10 H), 3.80(s, 3 H), 6.18-7.19(m, 4 H), 7.61(d, 1 H), 8.79(d, 1 H). |
| 67 | 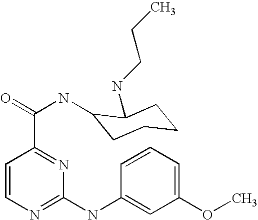 | 2-(3-methoxy-phenylamino)- pyrimidine-4-carboxylic acid(2- propylamino-cyclohexyl)-amide | ¹H NMR (CD₃OD, 300 MHz) δ 0.90-2.13(m, 6 H), 1.14-4.15(m, 10 H), 3.83(s, 3 H), 6.16-7.21(m, 4 H), 7.63(d, 1 H), 8.84(d, 1 H). |
| 68 | 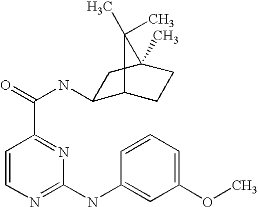 | 2-(3-methoxy-phenylamino)- pyrimidine-4-carboxylic acid(4,7,7-trimethyl- bicyclo[2,2,1]heptan-2-yl)- amide | ¹H NMR (CD₃OD, 300 MHz) δ 0.99-1.57(m, 18 H), 3.83(s, 3 H), 6.18-7.19(m, 4 H), 7.62(d, 1 H), 8.79(d, 1 H). |
| 69 | 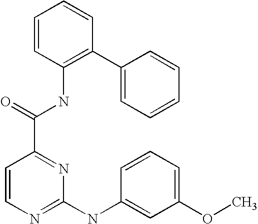 | 2-(3-methoxy- phenylamino)-pyrimidine- 4-carboxylic acidbiphenyl- 2-yl amide | ¹H NMR (CD₃OD, 300 MHz) δ 3.77(s, 3 H), 6.18-7.52(m, 13 H), 7.66(d, 1 H), 8.81(d, 1 H). |
| 70 | 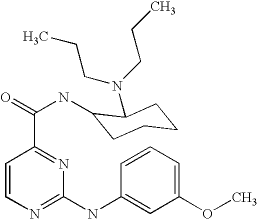 | 2-(3-methoxy- phenylamino)-pyrimidine- 4-carboxylic acid(2- dipropylamino- cyclohexyl)-amide | ¹H NMR (CD₃OD, 300 MHz) δ 0.90(t, 6 H), 1.43-3.77(m, 18 H), 3.83(s, 3 H), 6.12-7.21(m, 4 H), 7.59(d, 1 H), 8.77(d, 1 H). |
| 71 | 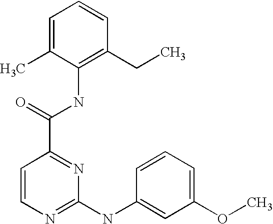 | 2-(3-methoxy- phenylamino)-pyrimidine- 4-carboxylic acid(2-ethyl- 5-methyl-phenyl)-amide | ¹¹H NMR (CD₃OD, 300 MHz) δ 1.32(q, 3 H), 2.12(m, 5 H), 3.83(s, 3 H), 6.18-7.48(m, 7 H), 7.59(s, 1 H), 8.77(s, 1 H). |
| 72 | 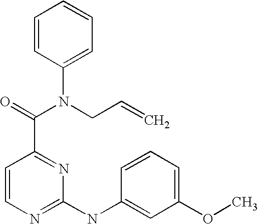 | 2-(3-methoxy- phenylamino)-pyrimidine- 4-carboxylic acid aryl- phenyl-amide | ¹¹H NMR (CD₃OD, 300 MHz) δ 3.83(s, 3 H), 4.90-7.81(m, 14 H), 7.63(s, 1 H), 8.87(s, 1 H). |
| 73 | 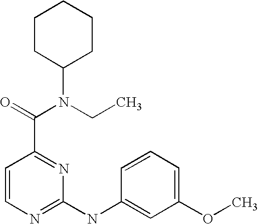 | 2-(3-methoxy- phenylamino)-pyrimidine- 4-carboxylic acid cyclohexyl-ethyl-amide | ¹¹H NMR (CD₃OD, 300 MHz) δ 1.11-3.54(m, 10 H), 1.35(t, 3 H), 3.75(q, 2 H), 3.83(s, 3 H), 6.12- 71.9(m, 4 H), 7.33(d, 1 H), 8.77(d, 1 H). |
| 74 | 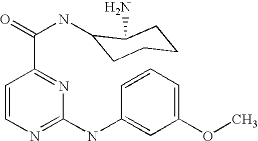 | 2-(3-methoxy- phenylamino)-pyrimidine- 4-carboxylic acid(2- amino-cyclohexyl)-amide | ¹H NMR (CD₃OD, 300 MHz) δ 1.21-3.77(m, 10 H), 3.38(s, 3 H), 6.12-7.23(m, 4 H), 7.63(d, 1 H), 8.82(d, 1 H). |
| 75 | 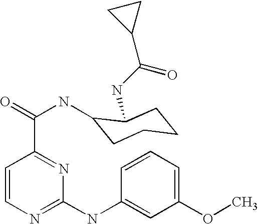 | 2-(3-methoxy- phenylamino)-pyrimidine- 4-carboxylic acid[2- (cyclopropanecarbonyl- amino)-cyclohexyl]-amide | ¹H NMR (CD₃OD, 300 MHz) δ 0.64-0.86(m, 4 H), 1.16(m, 1 H), 1.11-4.16(m, 10 H), 3.83(s, 3 H), 6.11-7.09(m, 4 H), 7.63(d, 1 H), 8.84(d, 1 H). |
| 76 | 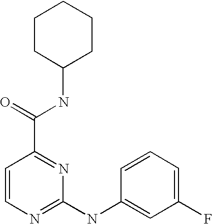 | 2-(3-fluoro-phenylamino)- pyrimidine-4-carboxylic acid cyclohexyl amide | ¹H NMR (CD₃OD, 300 MHz) δ 1.11-3.54(m, 10 H), 6.60-7.76(m, 4 H), 7.62(d, 1 H), 8.81(d, 1 H). |
| 77 | 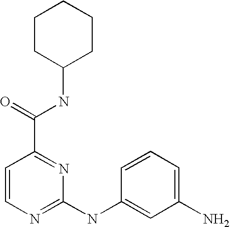 | 2-(3-amino-phenylamino)- pyrimidine-4-carboxylic acid cyclohexylamide | ¹H NMR (CD₃OD, 300 MHz) δ 1.11-3.54(m, 10 H), 6.54-7.55(m, 4 H), 7.61(d, 1 H), 8.84(d, 1 H). |
| 78 | 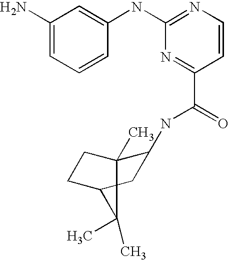 | 2-(3-amino-phenylamino)- pyrimidine-4-carboxylic acid(1,7,7-trimethyl- bicyclo[2,2,1]heptyl-2-yl)-amide | CD₃OD: 0.88(s, 3 H), 0.99(s, 3 H), 1.01(s, 3 H), 1.38-1.40(m, 2 H), 1.42- 1.60(m, 1 H), 1.69-1.79(m, 2 H), 1.82-1.93(m, 1 H), 2.38-2.58(m, 1 H), 4.35-4.42(m, 1 H), 6.98(d, J = 2.5 Hz, 1 H), 7.20-7.29(m, 2 H), 7.65(d, J = 2.5 Hz, 1 H), 8.03(s, 1 H), 8.79 (d, J = 2.4 Hz, 1 H) |
| 79 | 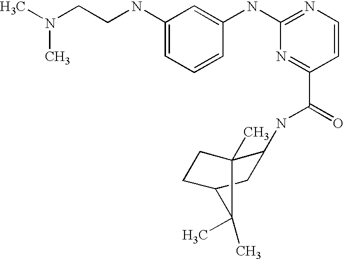 | 2-(3-(2- (dimethylamino)ethylamino) phenylamino)-N-(1,7,7- trimethylbicyclo[2.2.1]heptane- 2-yl)pyrimidine-4-carboxy- amide | CDCl₃: 0.86(s, 3 H), 0.91(s, 3 H), 1.00(s, 3 H), 1.24-1.88(m, 6 H), 2.31- 2.41(m, 1 H), 2.96(t, J = 5.4 Hz, 2 H), 3.37(t, J = 5.4 Hz, 2 H), 3.54(s, 6 H), 4.32-4.40(m, 1 H), 6.58(d, J = 7.5 Hz, 1 H), 6.78(s, 1 H), 7.09(d, J = 8.1 Hz, 1 H), 7.19(t, J = 7.8 Hz, 1 H), 7.21(s, 1 H), 7.45(d, J = 5.1 Hz, 1 H), 7.99(d, J = 9.61 Hz, 1 H), 8.60(d, J = 4.0 Hz, 1 H) |
| 80 | 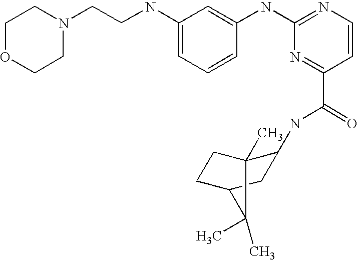 | 2-(3-(2- morpholinoethylamino) phenylamino)-N-(1,7,7- trimethylbicyclo[2.2.1] heptane-2- yl)pyrimidine-4- carboxyamide | CDCl₃: 0.86(s, 3 H), 0.91(s, 3 H), 1.00(s, 3 H), 1.24-1.85(m, 5 H), 2.38- 2.47(m, 1 H), 2.53(b, 4 H), 2.69(b, 2 H), 3.23(b, 2 H), 3.76(b, 4 H), 4.35- 4.40(m, 1 H), 6.40(d, J = 8.1 Hz, 1 H), 6.78(s, 1 H), 7.05(d, J = 7.8 Hz, 1 H), 7.16(t, J = 7.5 Hz, 2 H), 7.21(s, 1 H), 7.49(d, J = 4.8 Hz, 1 H), 7.99(d, J = 8.7 Hz, 1 H), 8.60(d, J = 4.5 Hz, 1 H) |
| 81 | 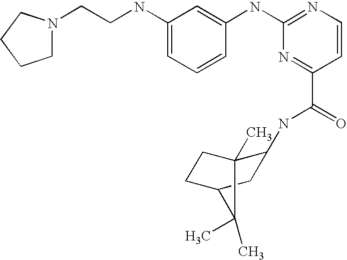 | 2-(3-(2-(pyridino-1- yl)ethylamino)phenylamino)-N- (1,7,7- trimethylbicyclo[2.2.1]heptane- 2-yl)pyrimidine-4-car- boxyamide | CDCl₃: 0.86(s, 3 H), 0.91(s, 3 H), 1.00(s, 3 H), 1.25-1.87(m, 6 H), 1.92(b, 4 H), 2.36-2.47(m, 1 H), 2.84(b, 4 h), 2.96(t, J = 5.1 Hz, 2 H), 3.37(t, J = 5.1 Hz, 2 H), 4.33-4.41(m, 1 H), 6.38(d, J = 7.5 Hz, 1 H), 6.78(s, 1 H), 7.09(d, J = 8.1 Hz, 1 H), 7.16(t, J = 7.8 Hz, 1 H), 7.21(s, 1 H), 7.47(d, J = 5.1 Hz, 1 H), 7.99(d, J = 9.61 Hz, 1 H), 8.60(d, J = 4.0 Hz, 1 H) |
| 82 | 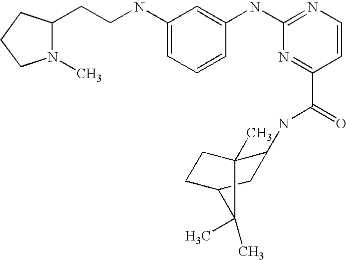 | 2-(3-(2-(1- methylpyrimidino-2- yl)ethylamino)phenylamino)- N-(1,7,7- trimethylbicyclo[2.2.1]heptane- 2-yl)pyrimidine-4- carboxyamide | CDCl₃: 0.86(s, 3 H), 0.92(s, 3 H), 0.99(s, 3 H), 1.24-1.85(m, 6 H), 1.92(b, 2 H), 2.36- 2.47(m, 2 H), 2.67(b, 2 H), 2.84(b, 2 H), 2.96(t, J = 5.1 Hz, 2 H), 3.01(s, 3 H), 3.37(t, J = 5.1 Hz, 2 H), 4.33-4.41(m, 1 H), 6.38(d, J = 7.5 Hz, 1 H), 6.78(s, 1 H), 7.09(d, J = 8.1 Hz, 1 H), 7.16(t, J = 7.8 Hz, 1 H), 7.21(s, 1 H), 7.47(d, J = 5.1 Hz, 1 H), 7.99(d, J = 9.61 Hz, 1 H), 8.60(d, J = 4.0 Hz, 1 H) |
| 83 | 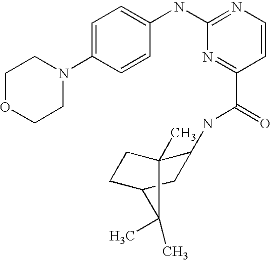 | 2-(4- morpholinophenylamino)- N-(1,7,7- trimethylbicyclo[2.2.1]heptane- 2-yl)pyrimidine-4- carboxyamide | CDCl₃: 0.86(s, 3 H), 0.93(s, 3 H), 1.01(s, 3 H), 1.19-1.83(m, 5 H), 2.01-2.03(m, 1 H), 2.52(b, 4 H), 3.00(b, 4 H), 4.30-4.44(m, 1 H), 6.63(d, J = 9.0 Hz, 2 H), 7.12(s, 1 H), 7.43(d, J = 9.0 Hz, 2 H), 7.44(s, 1 H), 7.99(d, J = 8.7 Hz, 2 H), 8.60(d, J = 5.1 Hz, 1 H) |
| 84 | 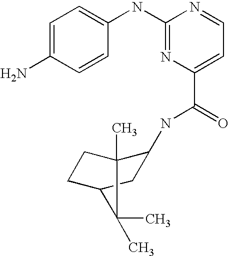 | 2-(4-aminophenylamino)-N- (1,7,7- trimethylbicyclo[2.2.1]heptane- 2-yl)pyrimidine-4- carboxyamide | CDCl₃: 0.88(s, 3 H), 0.91(s, 3 H), 0.99(s, 3 H), 1.20-1.83(m, 5 H), 2.35-2.44(m, 1 H), 2.61(t, J = 5.9 Hz, 2 H), 4.31-4.37(b, 1 H), 6.67(d, J = 8.7 Hz, 2 H), 7.02(s, 1 H), 7.21- 7.33(m, 2 H), 8.02(d, J = 9.0 Hz, 1 H), 8.56(d, J = 4.5 Hz, 1 H) |
| 85 | 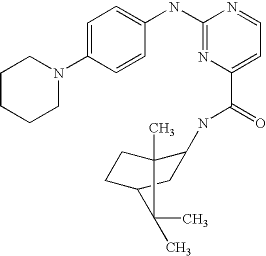 | 2-(4-(piperidine-1- yl)phenylamino)-N-(1,7,7- trimethylbicyclo[2.2.1]heptane- 2-yl)pyrimidine-4- carboxyamide | CDCl₃: 0.85(s, 3 H), 0.91(s, 3 H), 0.99(s, 3 H), 1.19-1.21(m, 2 H), 1.42-1.47(m, 1 H), 1.57-1.63(m, 1 H), 1.70-1.73(m, 1 H), 1.78- 1.83(m, 1 H), 2.01-2.03(m, 1 H), 2.22(b, 4 H), 2.60-2.65(m, 5 H), 4.30-4.38(m ,1 H), 6.93(d, J = 8.7 Hz, 2 H), 7.12(s, 1 H), 7.43(d, J = 8.7 Hz, 2 H), 7.44(s, 1 H), 7.90(d, J = 8.1 Hz, 2 H), 8.56(d, J = 4.8 Hz, 1 h) |
| 86 | 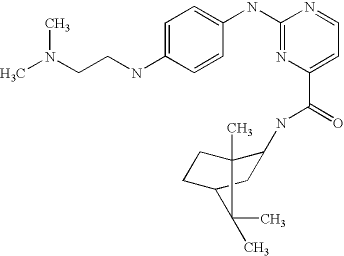 | 3-(4- dimethylaminoethylamino)- N-(1,7,7- trimethylbicyclo[2.2.1]heptane- 2-yl)pyrimidine-4- carboxyamide | CDCl₃: 0.88(s, 3 H), 0.91(s, 3 H), 0.99(s, 3 H), 1.20-1.28(m, 2 H), 1.37-1.48(m, 1 H), 1.57-1.66(m, 1 H), 1.70-1.73(m, 1 H), 1.78- 1.83(m, 1 H), 2.02-2.04(m, 1 H), 2.34(s, 6 H), 2.35-2.44(m, 1 H), 2.61(t, J = 5.9 Hz, 2 H), 3.17(t, J = 5.9 Hz, 2 H), 4.31-4.37(m, 1 H), 6.65(d, J = 8.7 Hz, 2 H), 7.05(s, 1 H), 7.29-7.40(m, 2 H), 8.02(d, J = 9.0 Hz, 1 H), 8.54(d, J = 4.5 Hz, 1 H) |
| 87 | 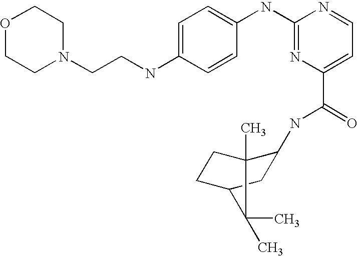 | 3-(4- morpholinophenylamino)- N-(1,7,7- trimethylbicyclo[2.2.1] heptane-2- yl)pyrimidine-4- carboxamide | CDCl₃: 0.86(s, 3 H), 0.91(s, 3 H), 0.99(s, 3 H), 1.22-1.27(m, 2 H), 1.37-1.45(m, 1 H), 1.54- 1.60(m, 1 H), 1.71-1.73(m, 1 H), 1.76-1.83(m, 1 H), 2.02-2.04(m, 1 H), 2.34(bs, 4 H), 2.35- 2.44(m, 1 H), 2.59(bs, 4 h), 2.61(t, J = 5.9 Hz, 2 H), 3.17(t, J = 5.9 Hz, 2 H), 4.31-4.37(m, 1 H), 6.64(d, J = 8.7 Hz, 2 H), 7.05(s, 1 H), 7.29- 7.40(m, 2 H), 8.02(d, J = 9.0 Hz, 1 H), 8.56(d, J = 4.5 Hz, 1 H) |
| 88 | 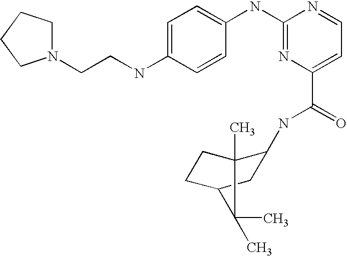 | 2-(4-(2-(pyrrolidine-1- yl)ethylamino)phenyl- amino)-N-(1,7,7- trimethylbicyclo[2.2.1] heptane-2- yl)pyrimidine- carboxamide | CD₃OD: 0.82(s, 3 H), 0.92(s, 3 H), 1.00(s, 3 H), 1.26-1.33(m, 3 H), 1.39-1.49(m, 1 H), 1.58- 1.72(m, 2 H), 1.80-1.85(m, 1 H), 2.00-2.08(m, 5 H), 2.35-2.43(m, 1 H), 3.41(bs, 3 H), 3.49(bs, 3 H), 4.29-4.30(m ,1 H), 6.71(bs, 2 H), 7.24(bs, 1 H), 7.38(d, J = 7.5 Hz, 2 H), 8.31(bs, 1 H), 8.56(bs, 1 H) |
| 89 | 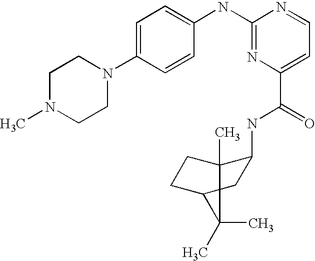 | 2-(4-(4- methylpiperazine-1- yl)phenylamino)-N- (1,7,7- trimethylbicyclo[2.2.1] heptane-2- yl)pyrimidine-4- carboxyamide | CDCl₃: 0.85(s, 3 H), 0.91(s, 3 H), 0.99(s, 3 H), 1.19-1.21(m, 2 H), 1.42-1.47(m, 1 H), 1.57- 1.63(m, 1 H), 1.70-1.73(m, 1 H), 1.78-1.83(m, 1 H), 2.01-2.03(m, 1 H), 2.38(s, 3 H), 2.62(b, 4 H), 3.02(b, 4 H), 4.30-4.38(m, 1 H), 6.93(d, J = 8.7 Hz, 2 H), 7.12(s, 1 H), 7.43(d, J = 8.7 Hz, 2 H), 7.44(s, 1 H), 7.99(d, J = 8.7 Hz, 2 H), 8.56(d, J = 5.1 Hz, 1 H) |
| 90 | 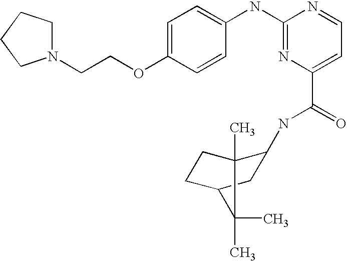 | 2-(4-(2-(piperidine-1- yl)ethoxy)phenylamino)- N-(1,7,7- trimethylbicyclo[2.2.1] heptane-2- yl)pyrimidine-4- carboxyamide | CD₃OD: 0.82(s, 3 H), 0.91(s, 3 H), 1.00(s, 3 H), 1.26-1.33(m, 3 H), 1.39-1.49(m, 1 H), 1.58- 1.72(m, 2 H), 1.80-1.85(m, 1 H), 2.00-2.08(m, 5 H), 2.35-2.43(m, 1 H), 3.41(bs, 3 H), 3.49(bs, 3 H), 4.29-4.30(m, 1 H), 6.71(bs, 2 H), 7.24(bs, 1 H), 7.38(d, J = 7.5 Hz, 2 H), 8.31(bs, 1 H), 8.56(bs, 1 H) |
| 91 | 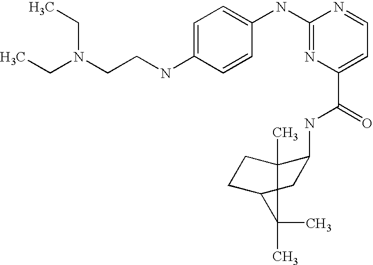 | 2-(4-(2- (diethylamino)ethylami- no)phenylamino)-N- (1,7,7- trimethylbicyclo[2.2.1] heptane-2- yl)pyrimidine-4- carboxamide | CD₃OD: 0.83(s, 3 H), 0.93(s, 3 H), 1.01(s, 3 H), 1.27-1.31(m, 6 H), 1.35-1.53(m, 1 H), 1.58- 1.72(m, 2 H), 1.80-1.91(m, 1 H), 1.94-2.01(m, 2 H), 2.34-2.43(m, 1 H), 2.70(m, 4 H), 3.16- 3.21(m, 2 H), 4.25-4.35(m, 1 H), 6.68(d, J = 8.7 Hz, 2 H), 7.23(d, J = 4.8 Hz, 1 H), 7.33(d, J = 8.7 Hz, 2 H), 8.54(d, J = 4.8 Hz, 1 H) |
| 92 | 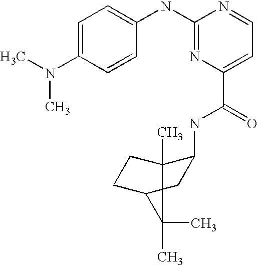 | 2-(4- (dimethylamino)ethylami- no)phenylamino)-N- (1,7,7- trimethylbicyclo[2.2.1]hep- tane-2-yl)pyrimidine-4- carboxyamide | CD₃OD: 0.73(s, 3 H), 0.75-0.82(m, 1 H), 0.84(s, 3 H), 0.91(s, 3 H), 1.35- 1.61(m, 3 H), 1.71-1.37(m, 2 H), 2.26- 2.30(m, 1 H), 3.21(s, 6 H), 4.19-4.21(m, 1 H), 6.71(b, 2 H), 7.14(s, 1 H), 7.29(d, J = 3.0 Hz, 2 H), 8.25(d, J = 3.5 Hz, 1 H), 8.60(d, J = 3.5 Hz, 1 H), |
| 93 | 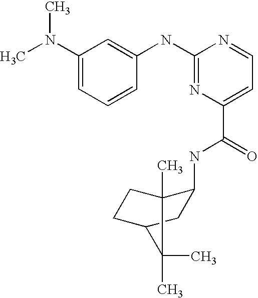 | 2-(3- (dimethylamino)ethylami- no)phenylamino)-N- (1,7,7- trimethylbicyclo[2.2.1]hep- tane-2-yl)pyrimidine-4- carboxyamide | CD₃OD: 0.80(s, 3 H), 0.87-0.94(m, 1 H), 0.89(s, 3 H), 0.98(s, 3 H), 1.22- 1.46(m, 2 H), 1.60-1.67(m, 2 H), 1.76- 1.83(m, 1 H), 2.32-2.40(m ,1 H), 2.90(s, 6 H), 4.28-4.33(m, 1 H), 6.46-6.49(m, 1 H), 6.89(s, 1 H), 7.11-7.12(m, 2 H), 7.27-7.29(m, 1 H), 8.56-8.58(m, 1 H) |
| 94 | 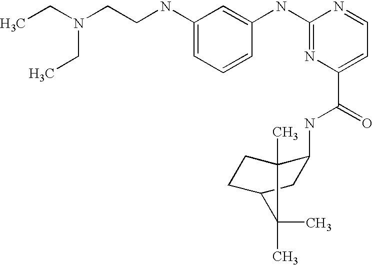 | 2-(3-(2- (diethylamino)ethylamino) phenylamino)-N- (1,7,7- trimethylbicyclo[2.2.1]hep- tane-2-yl)pyrimidine-4- carboxamide | CD₃OD: 0.87(s, 3 H), 0.88-0.91(m, 1 H), 0.92(s, 3 H), 1.00(s, 3 H), 1.26- 1.41(m, 6 H), 1.42-1.63(m, 2 H), 1.63- 1.85(m, 3 H), 2.38-2.42(m, 1 H), 3.21(s, 6 H), 4.19-4.21(m, 1 H), 6.71(b, 2 H), 7.14(s, 1 H), 7.29(d, J = 3.0 Hz, 2 H), 8.25(d, J = 3.5 Hz, 1 H), 8.60(d, J = 3.5 Hz, 1 H) |
| 95 | 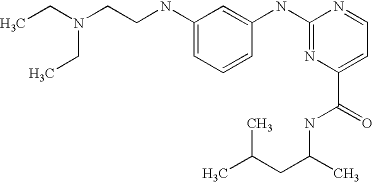 | 2-(3-(2- (diethylamino)ethylamino) phenylamino)-N-(4- methylpentane-2- yl)pyrimidine-4- carboxamide | CDCl₃: 0.91(d, 6 H), 1.02(q, 6 H), 1.32(d, 3 H), 1.47-1.62(m, 3 H), 2.46(t, J = 6.9 Hz, 4 H), 2.50(t, 2 H), 3.45(t, 2 H), 4.13(m, 1 H), 5.74-6.96(m, 4 H), 7.69(d, J = 8.1 Hz, 1 H), 9.01(d, J = 8.1 Hz, 1 H), |
| 96 | 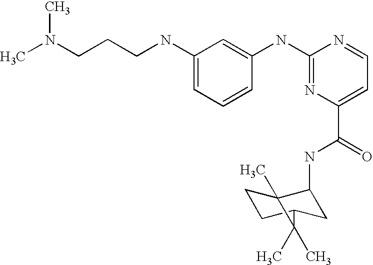 | 2-(3-(3- (phenylamino)phenylami- no)-N-(1,7,7- trimethylbicyclo[2.2.1]hep- tane-2-yl)pyrimidine-4- carboxyamide | CDCl₃: 0.86(s, 3 H), 0.91(s, 3 H), 0.99(s, 3 H), 1.25-1.73(m, 6 H), 1.79(d, J = 6.9 Hz, 2 H), 2.19-2.28(m, 1 H), 2.24(d, J = 6.9 Hz, 2 H), 3.19(d, J = 6.9 Hz, 2 H), 4.34-4.36(m, 2 H), 6.36(d, J = 8.1 Hz, 1 H), 6.71(s, 1 H), 7.05(d, J = 8.1 Hz, 1 H), 7.14(t, J = 7.5 Hz, 1 H), 7.24(s, 1 H), 7.54(t, J = 4.8 Hz, 1 H), 8.04(d, J = 9.6 Hz, 1 H), 8.60(d, J = 4.8 Hz, 1 H). |
| 97 | 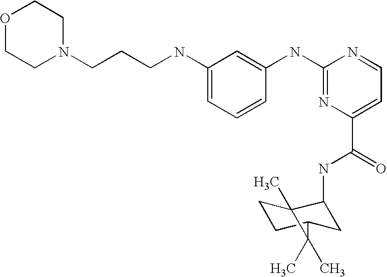 | 2-(3-(3- morpholinopropylamino) phenylamino)-N-(1,7,7- trimethylbicyclo[2.2.1]hep- tan-2-yl)pyrimidine-4- carboxyamide | CDCl₃: 0.86(s, 3 H), 0.91(s, 3 H), 0.99(s, 3 H), 1.25-1.73(m, 6 H), 1.79(d, J = 6.9 Hz, 2 H), 2.19-2.28(m, 1 H), 2.24(d, J = 6.9 Hz, 2 H), 3.19(d, J = 6.9 Hz, 2 H), 4.34-4.36(m, 2 H), 6.36(d, J = 8.1 Hz, 1 H), 6.71(s, 1 H), 7.05(d, J = 8.1 Hz, 1 H), 7.14(t, J = 7.5 Hz, 1 H), 7.24(s, 1 H), 7.54(t, J = 4.8 Hz, 1 H), 8.04(d, J = 9.6 Hz, 1 H), 8.60(d, J = 4.8 Hz, 1 H), |
| 98 | 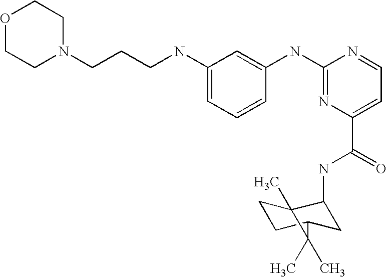 | 2-(3-(3- morpholinopropylamino) phenylamino)-N-(1,7,7- trimethylbicyclo[2.2.1]hep- tan-2-yl)pyrimidine-4- carboxyamide | CDCl₃: 0.86(s, 3 H), 0.91(s, 3 H), 1.00(s, 3 H), 1.21-1.85(m, 5 H), 1.84(d, J = 6.3 Hz, 2 H), 2.42-2.46(m, 1 H), 2.52(b, 6 H), 3.22(d, J = 6.3 Hz, 2 H), 3.76(b, 4 H), 4.35-4.40(m, 1 H), 6.36(d, J = 8.1 Hz, 1 h), 6.74(s, 1 H), 7.04(d, J = 8.1 Hz, 1 H), 7.14(t, J = 8.1 Hz, 2 H), 7.47(d, J = 4.8 Hz, 1 H), 8.00(d, J = 9.3 Hz, 1 H), 8.60(d, J = 4.8 Hz, 1 H) |
| 99 | 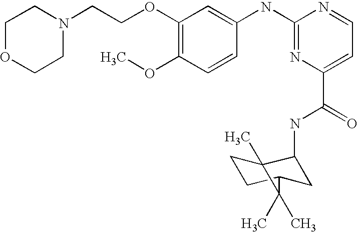 | 2-(4-methoxy-3-(2- morpholinoethoxy)phenyl- amino)-N-(1,7,7- trimethylbicyclo[2.2.1]hep- tan-2-yl)pyrimidine-4- carboxyamide | CDCl₃: 0.88(s, 3 H), 0.92(s, 3 H), 0.95-1.00(m, 1 H), 1.24(s, 3 H), 1.25-1.88(m, 5 H), 2.38-2.48(m, 2 H), 2.59(bs, 4 H), 2.78(t, J = 5.4 Hz, 2 H), 3.79(b, 4 H), 3.88(s, 4 H), 4.24(t, J = 5.4 Hz, 2 H), 4.34- 4.38(m, 1 H), 6.79-6.82(m, 1 H), 7.10(s, 1 H), 7.28-7.31(m, 1 H), 7.38-7.41(m, 1 H), 7.59(s, 1 H), 7.93-7.96(m, 1 H), 8.51-8.55(m, 1 H) |
| 100 | 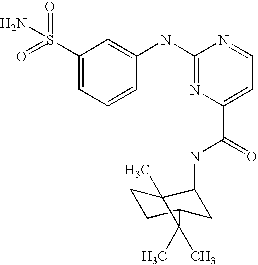 | 2-(3- sulfamoylphenylamino)- N-(1,7,7- trimethylbicyclo[2.2.1]hep- tan-2-yl)pyrimidine-4- carboxyamide | CDCl₃: 0.79(s, 3 H), 0.81(s, 3 H), 0.90(s, 3 H), 1.14-1.84(m, 5 H), 2.25-2.40(m, 1 H), 4.23-4.40(m, 1 H), 5.80(b, 2 H), 7.31-7.58(m, 3 H), 7.82-7.79(m, 2 H), 8.02(b, 1 H), 8.22(b, 1 H), 8.51(b, 1 H), |
| 101 | 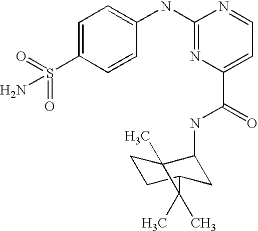 | 2-(4- sulfamoylphenylamino)- N-(1,7,7- trimethylbicyclo[2.2.1]hep- tan-2-yl)pyrimidine-4- carboxyamide | CDCl₃: 0.86(s, 3 H), 0.92(s, 3 H), 1.00(s, 3 H), 1.23-1.94(m, 5 H), 2.39-2.49(m, 1 H), 4.32-4.42(m, 1 H), 4.87(b, 2 H), 7.61(b, 2 H), 7.79(d, J = 8.7 Hz, 2 H), 7.92(d, J = 8.7 Hz, 2 H), 8.70(b, 1 H) |
| 102 | 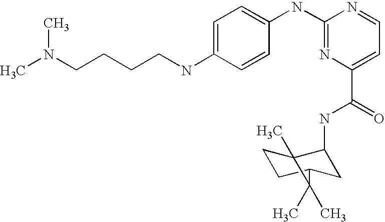 | 2-(4-(3- (dimethylamino)phenyl- amino)-N-(1,7,7- trimethylbicyclo[2.2.1]hep- tan-2-yl)pyrimidine-4- carboxyamide | CD₃OD: 0.83(s, 3 H), 0.93(s, 3 H), 1.01(s, 3 H), 1.27-1.31(m, 2 H), 1.35-1.53(m, 1 H), 1.58-1.72(m, 2 H), 1.80-1.91(m, 1 H), 1.94- 2.01(m, 2 H), 2.34-2.43(m, 1 H), 2.70(s, 6 H), 2.99-3.05(m, 2 H), 3.16-3.21(m, 2 H), 4.25-4.35(m, 1 H), 6.68(d, J = 8.7 Hz, 2 H), 7.23(d, J = 4.8 Hz, 1 H), 7.33(d, J = 8.7 Hz, 2 H), 8.54(d, J = 4.8 Hz, 1 H) |
| 103 | 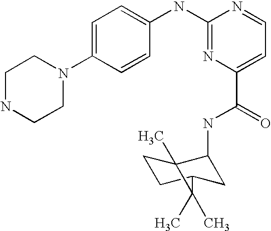 | 2-(4-(piperazin-1- yl)phenylamino)-N- (1,7,7- trimethylbicyclo[2.2.1]hep- tan-2-yl)pyrimidine-4- carobxyamide | CDCl₃: 0.85(s, 3 H), 0.91(s, 3 H), 0.99(s, 3 H), 1.19-1.21(m, 2 H), 1.42-1.47(m, 1 H), 1.57-1.63(m, 1 H), 1.70-1.73(m, 1 H), 1.78- 1.83(m, 1 H), 2.01-2.03(m, 1 H), 2.62(b, 4 H), 3.02(b, 4 H), 4.30- 4.38(m, 1 H), 6.93(d, J = 8.7 Hz, 2 H), 7.12(s, 1 H), 7.43(d, J = 8.7 Hz, 2 H), 7.44(s, 1 H), 7.99(d, J = 8.7 Hz, 2 H), 8.56(d, J = 5.1 Hz, 1 H) |
| 104 | 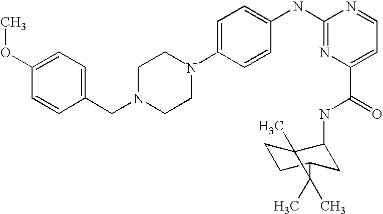 | 2-(4-(4-(4- methoxybenzyl)piperazin- 1-yl)phenylamino)-N- (1,7,7- trimethylbicyclo[2.2.1]hep- tan-2-yl)pyrimidine-4- carboxyamide | CDCl₃: 1.23-1.32(m, 3 H), 1.39- 1.61(m, 3 H), 1.68-1.92(m, 2 H), 2.28-2.35(m, 2 H), 2.70(bs, 3 H), 2.96(bs, 2 H), 3.23(bs, 4 H), 3.61(bs, 1 H), 3.82(s, 3 H), 3.81- 3.92(m, 1 H), 6.88-6.94(m, 4 H), 7.00(s, 1 H), 7.30-7.33(m, 2 H), 7.40-7.43(m, 2 H), 7.66-7.69(m, 2 H), 8.55(d, J = 5.1 Hz, 1 H) |
| 105 | 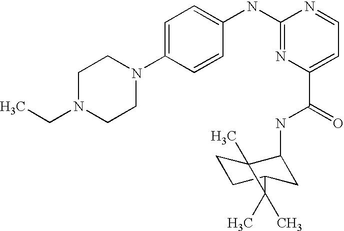 | 2-(4-(4-ethylpiperazin-1- yl)phenylamino)-N- (1,7,7- trimethylbicyclo[2.2.1]hep- tan-2-yl)pyrimidine-4- carboxyamide | CD₃OD: 0.83(s, 3 H), 0.94(s, 3 H), 1.01(s, 3 H), 1.15(t, J = 7.2 Hz, 3 H), 1.24-1.281(m, 1 H), 1.45-1.87(m, 4 H), 2.34-2.37(m, 1 H), 2.52(d, J = 7.2 Hz, 3 H), 2.67(d, J = 5.1 Hz, 4 H), 3.19(d, J = 5.1 Hz, 4 H), 4.29- 4.32(m, 1 H), 6.98(d, J = 9.0 Hz, 2 H), 7.27(d, J = 4.8 Hz, 2 H), 7.47(d, J = 9.0 Hz, 2 H), 8.58(d, J = 4.8 Hz, 2 H). |
| 106 | 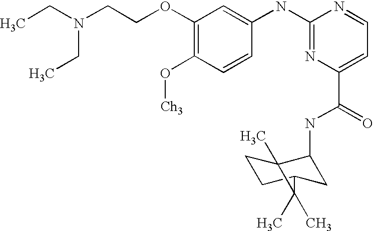 | 2-(3-(2- (diethylamino)ethoxy)-4- methoxyphenylamino)- N-(1,7,7- trimethylbicyclo[2.2.1]hep- tan-2-yl)pyrimidine-4- carboxyamide | CDCl₃: 0.80(s, 3 H), 0.85(s, 3 H), 0.94(s, 3 H), 1.18-1.25(m, 6 H), 1.92(b, 4 H), 1.29-1.42(m, 7 H), 1.54-1.67(m, 2 H), 1.69-1.79(m, 1 H), 2.22-2.38(m, 1 H), 3.12- 3.15(m, 4 H), 3.78(s, 3 H), 3.39- 4.40(m, 2 H), 6.79-6.82(m, 1 H0, 7.10(s, 1 H), 7.28-7.31(m, 1 H), 7.38-7.41(m, 1 H), 7.59(s, 1 H), 7.93-7.96(m, 1 H), 8.51-8.55(m, 1 H) |
| 107 | 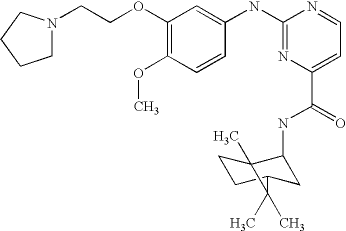 | 2-(4-methoxy-3-(2- (pyrrolidin-1- yl)ethoxy)phenylamino)- N-(1,7,7- trimethylbicyclo[2.2.1]hep- tan-2-yl)pyrimidine-4- carboxyamide | CDCl₃: CDCl3: 0.87(s, 3 H), 0.91(s, 3 H), 0.99-1.03(m, 1 H), 1.27(s, 3 H), 1.26-1.85(m, 5 H), 1.80(bs, 4 H), 2.98(t, J = 5.4 Hz, 2 H), 2.80(b, 4 H), 3.82(s, 3 H), 4.24(t, J = 5.4 Hz, 2 H), 4.34-4.38(m, 1 H), 6.79-6.82(m, 1 H), 7.10(s, 1 H), 7.28-7.31(m, 1 H), 7.38-7.41(m, 1 H), 7.59(s, 1 H), 7.93-7.96(m, 1 H), 8.51- 8.55(m, 1 H) |
| 108 | 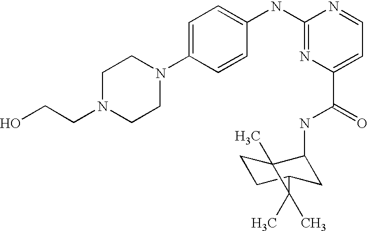 | 2-(4-(4-(2- hydroxyethyl)piperazin- 1-yl)phenylamino)-N- (1,7,7- trimethylbicyclo[2.2.1]hep- tan-2-yl)pyrimidine-4- carboxyamide | DMSO-d₆: 0.74(s, 3 H), 0.86(s, 3 H), 0.93(s, 3 H), 0.90-0.94(m, H), 1.22-1.75 (m, 5 H), 2.22-2.30(m, 1 H), 2.43(t, J = 6.3 Hz, 2 H), 2.53(bs, 4 H), 4.20- 4.28(m, 1 H), 4.38(t, J = 5.7 Hz, 1 H), 6.87(d, J = 9.0 Hz, 2 H), 7.16(d, J = 4.8 Hz, 1 H), 4.79(d, J = 9.0 Hz, 2 H), 8.07 (d, J = 9.6 Hz, 1 H), 8.60(d, J = 4.8 Hz, 1 H), 9.57(s, 1 H) |
| 109 | 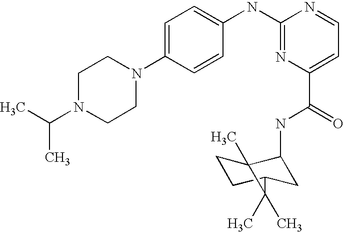 | 2-(4-(4- isopropylpiperazin-1- yl)phenylamino)-N- (1,7,7- trimethylbicyclo[2.2.1]hep- tan-2-yl)pyrimidine-4- carboxyamide | DMSO-d₆: 0.74(s, 3 H), 0.86(s, 3 H), 0.93(s, 3 H), 0.90-0.94(m, 1 H), 1.02(t, J = 6.3 Hz, 6 H), 1.19- 1.80(m, 5 H), 2.21-2.30(m, 1 H), 2.60(bs, 4 H), 3.07(bs, 4 H), 4.18- 4.25(m, 1 H), 6.87(d, J = 9.0 Hz, 2 H), 7.19(d, J = 4.8 Hz, 1 H), 7.49(d, J = 9.0 Hz, 2 H), 8.12(d, J = 9.6 Hz, 1 H), 8.45(d, J = 4.8 Hz, 1 H), 9.59(s, 1 H) |
| 110 | 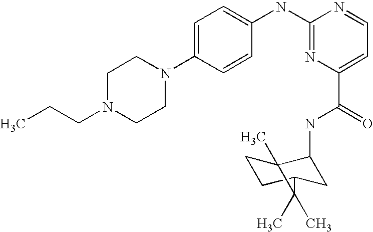 | 2-(4-(4-propylpiperazin- 1-yl)phenylamino)-N- (1,7,7- trimethylbicyclo[2.2.1]hep- tan-2-yl)pyrimidine-4- carboxyamide | CDCl₃: 0.85(s, 3 H), 0.91(s, 3 H), 0.95(t, J = 7.5 Hz, 3 H), 0.99(s, 3 H), 1.21-2.03(m, 8 H), 2.42-2.46(m, 3 H), 2.72(b, 4 H), 3.26(b, 4 H), 4.30-4.40(m, 1 H), 6.93(d, J = 9.0 Hz, 2 H), 7.06(s, 1 H), 7.43(d, J = 9.0 Hz, 2 H), 7.44(s, 1 H), 7.99(d, J = 8.7 Hz, 2 H), 8.56(d, J = 4.8 Hz, 1 H) |
| 111 | 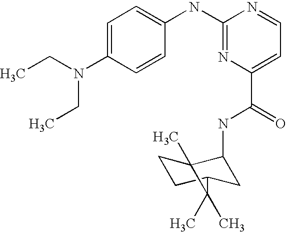 | 2-(4- (diethylamino)phenylami- no)-N-(1,7,7- trimethylbicyclo[2.2.1]hep- tan-2-yl)pyrimidine-4- carboxyamide | CDCl₃: 0.84(s, 3 H), 0.87-0.95(m, 1 H), 0.90(s, 3 H), 0.99(s, 1 H), 1.41-1.81(m, 5 H), 2.32-2.41(m, 1 H), 3.33(bs, 4 h), 4.22-4.38(m, 1 H), 6.68(b, 2 H), 7.35(b, 4 H), 8.05(d, J = 9.0 Hz, 1 H), 8.54(d, J = 4.2 Hz, 1 H) |
| 112 | 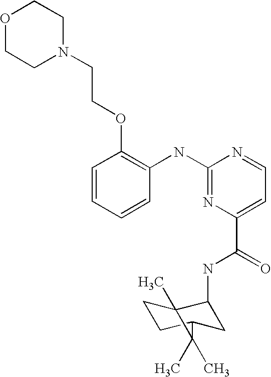 | 2-(2-(2- morpholinoethoxy)phenyl- amino)-N-(1,7,7- trimethylbicyclo [2.2.1]heptan-2- yl)pyrimdiine-4- carboxyamide | CDCl₃: 0.88(s, 3 H), 0.92(s, 3 H), 0.95-1.00(m, 1 H), 1.24(s, 3 H), 1.25-1.88(m, 5 H), 2.38-2.48(m, 2 H), 2.59(bs, 4 H), 2.78(t, J = 5.4 Hz, 2 H), 3.79(b, 4 H), 4.24(t, J = 5.4 Hz, 2 H), 4.34- 4.38(m, 1 H), 6.99-7.04(m, 3 H), 7.48(d, J = 4.8 Hz, 1 H), 8.03(d, J = 9.6 Hz, 1 H), 8.27(t, J = 4.2 Hz, 1 H), 8.37(s, 1 H), 8.62(d, J = 5.1 Hz, 1 H) |
| 113 | 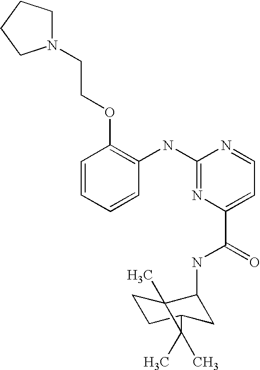 | 2-(2-(2-(pyrrolidin-1- yl)ethoxy)phenylamino)- N-(1,7,7-trimethylbicyclo [2.2.1]heptan-2- yl)pyrimidine-4- carboxyamide | CDCl₃: 0.87(s, 3 H), 0.91(s, 3 H), 0.99-1.03(m, 1 H), 1.27(s, 3 H), 1.26-1.85(m, 5 H), 1.80(bs, 4 H), 2.98(t, J = 5.4 Hz, 2 H), 2.80(b, 4 H), 4.24(t, J = 5.4 Hz, 2 H), 4.34- 4.38(m, 1 H), 6.97-7.02(m, 3 H), 7.47(d, J = 4.8 Hz, 1 H), 8.13(d, J = 9.6 Hz, 1 H), 8.20(t, J = 4.2 Hz, 1 H), 8.35(s, 1 H), 8.60(d, J = 5.1 Hz, 1 H) |
| 114 | 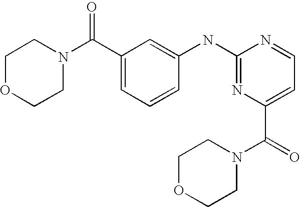 | 2-(3-(morpholine-4- carbonyl)phenylamino)pyri- midine-4- yl)(morpholino)methanone | CDCl₃: 3.58-3.80(m, 16 H), 6.98(d, J = 5.1 Hz, 1 H), 7.08(d, J = 7.2 Hz, 1 H), 7.36(d, J = 7.8 Hz, 1 H), 7.42(b, 1 H), 7.61(d, J = 8.1 Hz, 1 H), 7.77(b, 1 H), 8.56(d, J = 4.5 Hz, 1 H) |
| 115 | 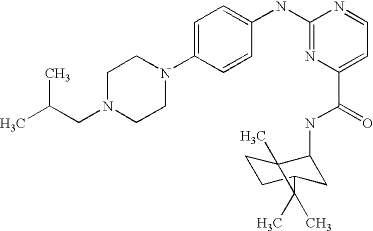 | 2-(4-(4-isobutylpiperazin- 1-yl)phenylamino)-N- (1,7,7-trimethylbicyclo- [2.2.1]heptan-2- yl)pyrimidine-4- carboxyamide | CDCl₃: 0.86(s, 3 H), 0.92(s, 3 H), 1.00(s, 3 H), 1.20-1.87(m, 7 H), 1.56(b, 6 H), 2.39-2.43(m, 1 H), 3.00(b, 4 H), 3.46(b, 4 H), 4.30- 4.38(m, 1 H), 6.94(d, J = 9.0 Hz, 2 H), 7.06(s, 1 H), 7.46(d, J = 9.0 Hz, 2 H), 7.97(d, J = 9.0 Hz, 2 H), 8.58(d, J = 4.8 Hz, 1 H) |
| 116 | 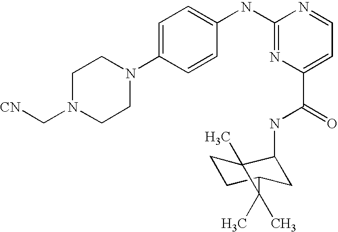 | 2-(4-(4- (cianomethyl)piperazin-1- yl)phenylamino)-N-(1,7,7- trimethylbicyclo [2.2.1]heptan-2- yl)pyrimidine-4- carboxyamide | CDCl₃: 0.86(s, 3 H), 0.92(s, 3 H), 1.00(s, 3 H), 1.20-1.85(m, 6 H), 2.39-2.42(m, 1 H), 2.84(b, 4 H), 3.25(b, 4 H), 3.58(s, 2 H), 4.31- 4.38(m, 1 H), 6.88-6.94(m, 4 H), 7.02(b, 2 H), 7.14(b, 1 H), 7.40- 7.43(d, J = 9.3 Hz, 1 H), 8.57(b, 1 H) |
| 117 | 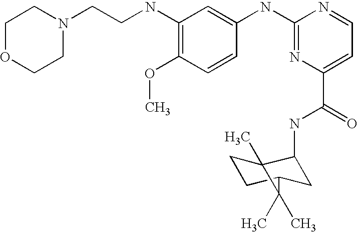 | 2-(4-methoxy-3-(2- morpholinoethylamino)phenyl- amino)-N-(1,7,7- trimethylbicyclo- [2.2.1]heptane-2- yl)pyrimidine-4- carboxyamide | CDCl₃: 0.86(s, 3 H), 0.91(s, 3 H), 1.00(s, 3 H), 1.26(bs, 2 H), 1.38-1.45(m, 1 H), 1.57- 1.68(m, 1 H), 1.70-1.74(m, 1 H), 1.77- 1.84(m, 1 H), 1.88-2.09(m, 1 H), 2.36- 2.43(m, 2 H), 2.52(bs, 4 H), 2.70(t, J = 4.5 Hz, 2 H), 3.20-3.22(m, 2 H), 3.74(s, 4 H), 3.86(s, 3 H), 4.31-4.42(m, 1 H), 6.61(s, 1 H), 6.74(d, J = 8.4 Hz, 1 H), 6.97-7.04(m, 2 H), 7.42- 7.44(m, 1 H), 8.02(d, J = 4.5 Hz, 1 H), 8.57(d, J = 4.8 Hz, 1 H) |
| 118 | 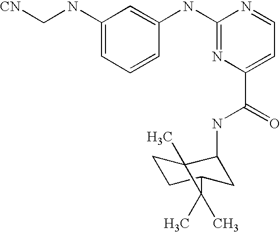 | 2-(3- (cianomethylamino)phenylami- no)-N-(1,7,7- trimethylbicyclo- [2.2.1]heptane-2- yl)pyrimidine-4- carboxyamide | CDCl₃: 0.80(s, 3 H), 0.85(s, 3 H), 0.94(s, 3 H), 1.18-1.25(m, 6 H), 1.29-1.42(m, 5 H), 1.54-1.67(m, 2 H), 2.22-2.38(m, 1 H), 4.33- 4.40(m, 1 H), 6.79-6.82(m, 1 H), 7.10(s, 1 H), 7.28-7.31(m, 1 H), 7.38-7.41(m, 1 H), 7.59(s, 1 H), 7.93-7.96(m, 1 H), 8.51-8.55(m, 1 H) |
| 119 | 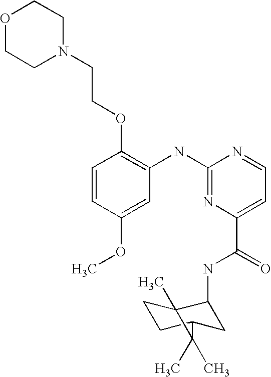 | 2-(5-methoxy-2-(2- morpholinoethoxy)phenylami- no)-N-(1,7,7-trimethylbicyclo [2.2.1]heptane-2- yl)pyrimidine-4- carboxyamide | CDCl₃: 0.88(s, 3 H), 0.92(s, 3 H), 0.95- 1.00(m, 1 H), 1.24(s, 3 H), 1.25-1.88(m, 5 H), 2.38-2.48(m, 2 H), 2.59(bs, 4 H), 2.78(t, J = 5.4 Hz, 2 H), 3.79(b, 4 H), 3.86(s, 3 H), 4.24(t, J = 5.4 Hz, 2 H), 4.34-4.38(m, 1 H), 6.99-7.04(m, 3 H), 7.48(d, J = 4.8 Hz, 1 H), 8.03(d, J = 9.6 Hz, 1 H), 8.27(t, J = 4.2 Hz, 1 H), 8.37(s, 1 H), 8.62(d, J = 5.1 Hz, 1 H) |
| 120 | 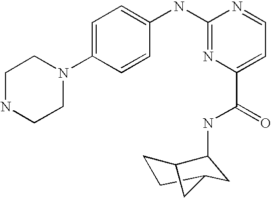 | N-(bicyclo[2.2.1]heptane-2- yl)-2-(4-(piperazine-1- yl)phenylamino)-N-(1,7,7- trimethylbicyclo- [2.2.1]heptane-2- yl)pyrimidine-4- carboxyamide | CDCl₃: 0.83-0.87(m, 1 H), 1.24-1.62(m, 6 H), 1.91-2.03(m, 2 H), 2.11-2.14(m, 1 H), 2.48(s, 1 H), 3.06(b, 4 H), 3.10(b, 4 H), 4.28(b, 1 H), 6.93(d, J = 8.1 Hz, 2 H), 7.32(s, 1 H), 7.42(d, J = 8.1 Hz, 2 H), 7.43(d, J = 6.6 Hz, 1 H), 7.94(d, J = 7.8 Hz, 1 H), 8.55(d, J = 6.6 Hz, 1 H), |
| 121 | 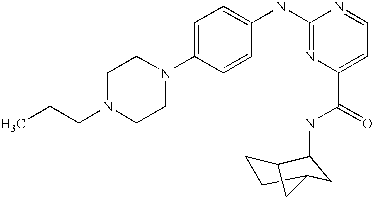 | N-(bicyclo[2.2.1]heptane-2- yl)-2-(4-(4-propylpiperazine- 1-yl)phenylamino)-N-(1,7,7- trimethylbicyclo- [2.2.1]heptane-2- yl)pyrimidine-4- carboxyamide(endo) | CDCl₃: 0.84-0.87(m, 1 H), 0.94(t, J = 7.5 Hz, 3 H), 1.25-1.52(m, 6 H), 1.57-1.69(m, 4 H), 2.15-2.20(m, 1 H), 2.41(b, 2 H), 2.67(b, 4 H), 3.22(b, 4 H), 4.28(b, 1 H), 6.94(d, J = 9.0 Hz, 2 H), 7.03(s, 1 H), 7.42(d, J = 9.0 Hz, 2 H), 7.43(s, 1 H), 7.93(d, J = 7.5 Hz, 1 H), 8.56(d, J = 4.5 Hz, 1 H), |
| 122 | 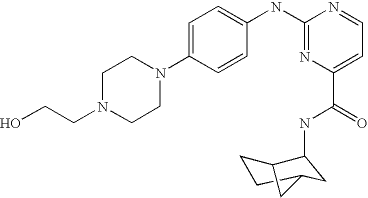 | N- (bicyclo[2.2.1]heptane- 2-yl)-2-(4-(4-hydroxy propylpiperazine-1- yl)phenylamino)-N- (1,7,7- trimethylbicyclo[2.2.1] heptane-2- yl)pyrimidine-4- carboxyamide(endo) | CDCl₃: 1.18-1.32(m, 2 H), 1.40-1.44(m, 1 H), 1.48-1.57(m, 2 H), 1.83-1.94(m, 1 H), .30-2.34(m, 2 H), 2.75(t, J = 5.1 Hz, 2 H), 2.85(bs, 4 H), 3.14-3.19(m, 1 H), 3.28(t, J = 4.8 Hz, 4 H), 3.76(t, J = 5.1 Hz, 2 H), 3.83-3.91(m, 1 H), 6.94(d, J = 9.0 Hz, 2 H), 7.04(s, 1 H), 7.42-7.45(m, 3 H), 7.68(d, J = 4.8 Hz, 1 H), 8.56(d, J = 4.5 Hz, 1 H) |
| 123 | 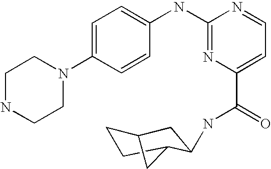 | N- (bicyclo[2.2.1]heptane- 2-yl)-2-(4-(piperazine- 1- yl)phenylamino)pyrimi- dine-4-carboxyamide | CDCl₃: 1.12-1.19(m, 1 H), 1.22-1.31(m, 3 H), 1.36-1.43(m, 1 H), 1.44-1.59(m, 2 H), 1.82-.96(m, 2 H), 2.28-2.31(m, 2 H), 3.05(bs, 4 H), 3.09(bs, 4 H), 3.84-3.88(m, 1 H), 6.91(d, J = 9.0 Hz, 2 H), 7.39-7.42(m, 3 H), 7.52(s, 1 H), 7.69(d, J = 8.2 Hz, 1 H), 8.52(d, J = 4.8 Hz, 1 H) |
| 124 | 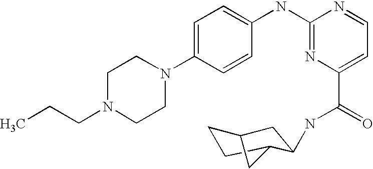 | N- (bicyclo[2.2.1]heptane- 2-yl)-2-(4-(4-4- propylpiperazine-1- yl)phenylamino)pyrimi- dine-4- carboxyamide(exo) | CDCl₃: 0.92-0.97(m, 3 H), 1.25-1.31(m, 3 H), 1.38-1.44(m, 1 H), 1.49-1.67(m, 4 H), 1.82-.94(m, 1 H), 2.30-2.36(m, 2 H), 2.44(t, J = 5.2 Hz, 2 H), 2.70(bs, 4 H), 3.24(t, J = 4.8 Hz, 4 H), 3.84-3.91(m, 1 H), 6.94(d, J = 9.3 Hz, 2 H), 7.07(s, 1 H), 7.40- 7.43(m, 3 H), 7.35(d, J = 4.8 Hz, 1 H), 8.55(d, J = 5.1 Hz, 1 H) |
| 125 | 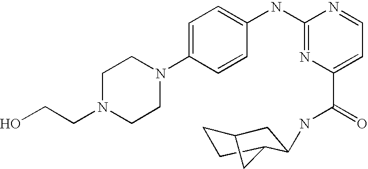 | N- (bicyclo[2.2.1]heptane- 2-yl)-2-(4-(4-(2- hydroxyethyl)piperazine- 1- yl)phenylamino)pyrimi- dine-4-carboxyamide | CDCl₃: 1.23-1.35(m, 3 H), 1.37-1.42(m, 1 H), 1.47-1.65(m, 4 H), 1.80-1.91(m, 1 H), 2.31-.39(m, 2 H), 2.47(t, J = 5.8 Hz, 2 H), 2.77(bs, 4 h), 3.21(t, J = 4.8 Hz, 4 H), 3.80-3.88(m, 1 H), 6.95(d, J = 9.3 Hz, 2 H), 7.00(s, 1 H), 7.45-7.48(m, 3 H), 7.65(d, J = 4.8 Hz, 1 H), 8.60(d, J = 5.1 Hz, 1 H) |
| 126 | 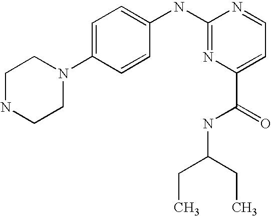 | N-(pentane-3-yl)-2-(4- (piperazine-1- yl)phenylamino)pyrimi dine-4-carboxyamide | CDCl₃: 0.94(t, J = 7.5 Hz, 6 H), 1.51(q, J = 7.2 Hz, 2 H), 1.65(q, J = 7.2 Hz, 2 H), 3.13(b, 4 H), 3.17(b, 4 H), 3.81(b, 1 H), 3.92-3.95(m, 1 H), 6.94(d, J = 8.7 Hz, 2 H), 7.06(s, 1 H), 7.42-7.45(m, 3 H), 7.57(d, J = 9.0 Hz, 2 H), 8.58(d, J = 5.1 Hz, 1 H) |
| 127 | 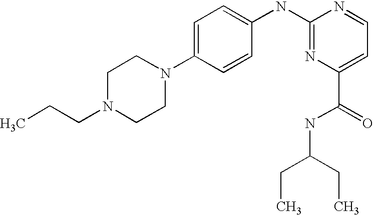 | N-(pentane-3-yl)-2-(4- (4-propylpiperazine-1- yl)phenylamino)pyrimi dine-4-carboxyamide | CDCl₃: 0.93(t, J = 7.5 Hz, 3 H), 1.13(t, J = 6.9 Hz, 3 H), 1.24(t, J = 7.2 Hz, 3 H), 1.54-1.60(m, 2 H), 2.40(t, J = 7.6 Hz, 2 H), 2.66(bs, 4 H), 3.19-3.22(m, 4 H), 3.32(q, 2 H), 3.52(q, 2 H), 6.78(d, J = 5.1 Hz, 1 H), 6.90-6.93(m, 1 H), 7.05(s, 1 H), 7.44(d, J = 8.7 Hz, 2 H), 8.28(d, J = 4.2 Hz, 1 H) |
| 128 | 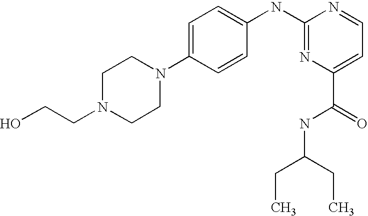 | 2-(4-(4-(2- hydroxyethyl)piperazine- 1-yl)phenylamino)-N- (pentane-3- yl)pyrimidine-4- carboxyamide | CDCl₃: 1.14(t, J = 7.2 Hz, 3 H), 1.25(t, J = 7.0 Hz, 3 H), 1.98-2.07(m, 1 H), 2.72(t, J = 4.5 Hz, 2 H), 2.80-2.83(m, 4 H), 3.23- 3.27(m, 4 H), 3.30-3.35(m, 2 H), 3.50- 3.57(m, 2 H), 3.71-3.76(m, 2 H), 6.79(d, J = 4.8 Hz, 1 H), 6.92(d, J = 9.0 Hz, 2 H), 7.02(s, 1 H), 7.47(d, J = 9.0 Hz, 2 H), 8.43(d, J = 4.5 Hz, 1 H) |
| 129 | 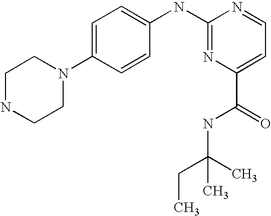 | N-tert-phenyl-2-(4- (piperazine-1- yl)phenylamino)pyrimi- dine-4-carboxyamide | CDCl₃: 0.91(t, J = 7.5 Hz, 3 H), 1.41(s, 6 H), 1.81(q, J = 7.2 Hz, 2 H), 3.16(b, 4 H), 3.20(b, 4 H), 6.94(d, J = 9.0 Hz, 2 H), 7.04(s, 1 H), 7.42-7.46(m, 3 H), 7.70(b, 1 H), 8.55-8.56(m, 1 H) |
| 130 | 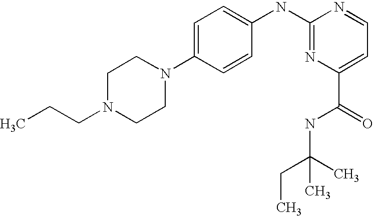 | 2-(4-(4- propylpiperazine-1- yl)phenylamino)-N- tert-pentylpyrimidine- 4-carboxyamide | CDCl₃: 0.84-0.97(m, 6 H), 1.25(s, 6 H), 1.60(b, 2 H), 1.74-1.82(m, 2 H), 2.02(b, 2 H), 2.69(b, 4 H), 3.23(b, 4 H), 6.91- 6.96(m, 2 H), 7.03(s, 1 H), 7.40-7.44(m, 3 H), 7.71(b, 1 H), 8.54-8.56(m, 1 H) |
| 131 | 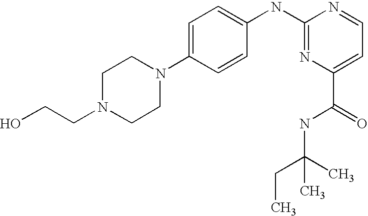 | 2-(4-(4-(2- hydroxyethyl)piperazine- 1-yl)phenylamino)-N- tert-pentylpyrimidine- 4-carbxoyamide | CDCl₃: 0.88-0.93(m, 3 H), 1.25(s, 6 H), 1.78-1.82(m, 2 H), 2.35(b, 1 H), 2.76(b, 2 H), 2.87(b, 4 H), 3.28(b, 4 H), 3.76(b, 2 H), 6.92-6.95(m, 2 H), 7.02(s, 1 H), 7.42-7.45(m, 3 H), 7.70(b, 1 H), 8.54- 8.56(m, 1 H) |
| 132 | 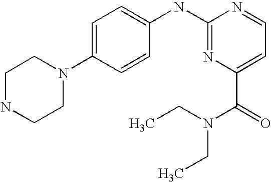 | N,N-diethyl-2-(4- (piperazine-1- yl)phenylamino)pyrimi- dine-4-carboxyamide | CDCl₃: 1.10(t, J = 6.9 Hz, 3 H), 1.21(t, J = 6.9 Hz, 3 H), 3.01(s, 4 H), 3.06(s, 4 H), 3.25-3.31(m, 2 H), 3.43-3.51(m, 2 H), 6.75(d, J = 4.5 Hz, 1 H), 6.88(d, J = 8.7 Hz, 2 H), 7.41-7.47(m, 3 H), 8.42(d, J = 4.8 Hz, 1 H) |
| 133 | 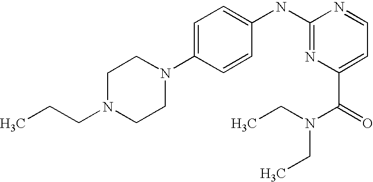 | N,N-diethyl-2-(4-(4- propylpiperazine-1- yl)phenylamino)pyrimidine- 4-carboxyamide | CDCl₃: 1.10(t, J = 6.9 Hz, 3 H), 1.21(t, J = 6.9 Hz, 3 H), 1.30(t, J = 6.9 Hz, 3 H), 2.30(d, J = 6.0 Hz, 3 H), 2.68(bs, 4 H), 3.00(bs, 4 H), 3.21(d, J = 6.0 Hz, 3 H), 3.25-3.31(m, 2 H), 3.43-3.51(m, 2 H), 6.73(d, J = 4.5 Hz, 1 H), 6.86(d, J = 8.7 Hz, 2 H), 7.41-7.47(m, 3 H), 8.60(d, J = 4.8 Hz, 1 H) |
| 134 | 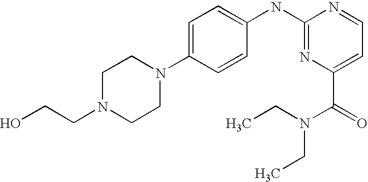 | N,N-diethyl-2-(4-(4-(2- hydroxyethyl)piperazine-1- yl)phenylamino)pyrimidine- 4-carboxyamide | CDCl₃: 1.10(t, J = 6.9 Hz, 3 H), 1.21(t, J = 6.9 Hz, 3 H), 2.59(bs, 1 H), 2.67(d, J = 2.1 Hz, 3 H), 2.68(bs, 4 H), 3.00(bs, 4 H), 3.70(d, J = 2.1 Hz, 2 H), 3.25- 3.31(m, 2 H), 3.43-3.51(m, 2 H), 6.73(d, J = 4.5 Hz, 1 H), 6.86(d, J = 8.7 Hz, 2 H), 7.41-7.47(m, 3 H), 8.59(d, J = 4.8 Hz, 1 H) |
| 135 | 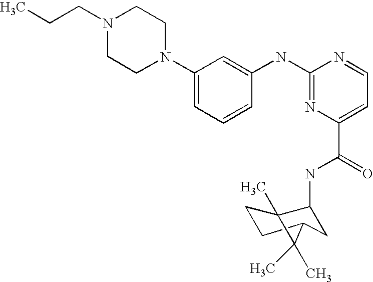 | 2-(3-(4-propylpiperazine-1- yl)phenylamino)-N-(1,7,7- trimethylbicyclo[2.2.1]heptane- 2-yl)pyrimidine-4- carboxyamide | CDCl₃: 0.85(s, 3 H), 0.90(s, 3 H), 0.93(t J = 7.5 Hz, 2 H), 0.98(s, 3 H), 1.21-1.82(m, 7 H), 2.35-2.44(m, 1 H), 2.37(d, J = 8.1 Hz, 2 H), 2.63(bs, 4 H), 3.25(bs, 4 H), 4.35- 4.36(m, 1 H), 6.67(d, J = 7.5 Hz, 1 H), 6.94(s, 1 H), 7.25(t, J = 7.8 Hz, 2 H), 7.31(s, 1 H), 7.47(d, J = 4.8 Hz, 1 H), 7.97(d, J = 9.3 Hz, 1 h), 8.60(d, J = 4.8 Hz, 1 H) |
| 136 | 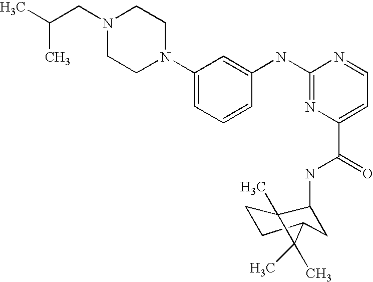 | 2-(3-(4-isobutylpiperazine- 1-yl)phenylamino)-N- (1,7,7- trimethylbicyclo[2.2.1]heptane- 2-yl)pyrimidine-4- carboxyamide | CDCl₃: 0.86(s, 3 H), 0.91(s, 3 H), 0.93(s, 3 H), 0.95(s, 3 H), 0.99(s, 3 H), 1.28- 1.31(m, 2 H), 1.43-1.48(m, 1 H), 1.58- 1.64(m, 1 H), 1.67-1.74(m, 1 H), 1.78- 1.87(m, 2 H), 2.19(d, J = 7.2 Hz, 2 H), 2.36-2.45(m, 1 H), 2.60(bs, 4 H), 3.25(bs, 4 H), 4.34-4.37(m, 1 H), 6.67-6.69(m, 1 H), 6.96(s, 1 H), 7.20-7.26(m, 1 H), 7.35(s, 1 H), 7.48(d, J = 4.8 Hz, 1 H), 7.98(d, J = 9.3 Hz, 1 H), 8.60(d, J = 4.8 Hz, 1 H) |
| 137 | 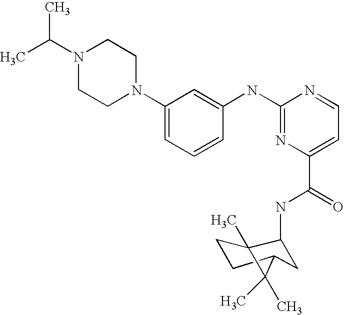 | 2-(3-(4-isopropylpiperazine- 1-yl)phenylamino)-N- (1,7,7- trimethylbicyclo[2.2.1]heptane- 2-yl)pyrimidine-4- carboxyamide | CDCl₃: 0.86(s, 3 H), 0.91(s, 3 H), 0.99(s, 3 H), 1.12(s, 3 H), 1.14(s, 3 H), 1.23- 1.31(m, 2 H), 1.42-1.49(m, 1 H), 1.59- 1.65(m, 3 H), 1.78-1.84(m, 1 H), 2.36- 2.45(m, 2 H), 2.72-2.75(m, 4 H), 3.26- 3.29(m, 4 H), 4.34-4.38(m, 1 H), 6.68(d, J = 7.5 Hz, 1 H), 6.97(s, 1 H), 7.20-7.29(m, 2 H), 7.48(d, J = 4.5 Hz, 1 H), 7.97(d, J = 9.3 Hz, 1 H), 8.61(d, J = 4.8 Hz, 1 H) |

The compound of formula 1 of the present invention may be in the form of a pharmaceutically acceptable salt derived from an inorganic or organic acid, or a base, and representative examples of the pharmaceutically acceptable salt derived from an inorganic or organic may include the known salts obtained by adding an inorganic acid such as hydrochloric acid, hydrobromic acid, phosphoric acid or sulfonic acid, or organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid or malic acid, methanesulfonic acid, or para toluenesulfonic acid, which do not limit its scope, to the compound of formula 1. Such acid salts may be prepared by the conventional processes, and other acids, which themselves are not pharmaceutically acceptable, including oxalic acid may be employed in the preparation of the bases.

Further, the compound of formula 1 may be used in the form of a derivative or prodrug thereof, wherein the derivative or prodrug thereof may be a physiologically hydrolysable ester or amide compound, e.g., indanyl, phthalidil, methoxymethyl, pivaloyloxymethyl, glycyloxymethyl, phenylglycyloxymethyl and 5-methyl-2-oxo-1,3-dioxolene-4-ylmethyl.

In accordance with another aspect of the present invention, there is provided a method for preparing the compound of formula 1.

A compound of formula 1 wherein R₁ is H may be prepared by a method comprising the steps of:

1) reacting a compound of formula 2 with NaNO₂ in a solvent to obtain a compound of formula 3;

2) reacting the compound of formula 3 with a halogenating agent to obtain a compound of formula 4;

3) reacting of the compound of formula 4 and a compound of formula 5 in a solvent under microwave irradiation to obtain a compound of formula 6;

4) dissolving the compound of formula 6 in an alkali hydroxide, refluxing the resulting mixture, and adding a strong acid thereto until pH of the mixture becomes 3, to obtain a compound of formula 7; and

5) subjecting the compound of formula 7 an amidation reaction with a compound of formula R₂R₃NH in a solvent in the presence of a coupling agent to obtain the compound of formula 1:

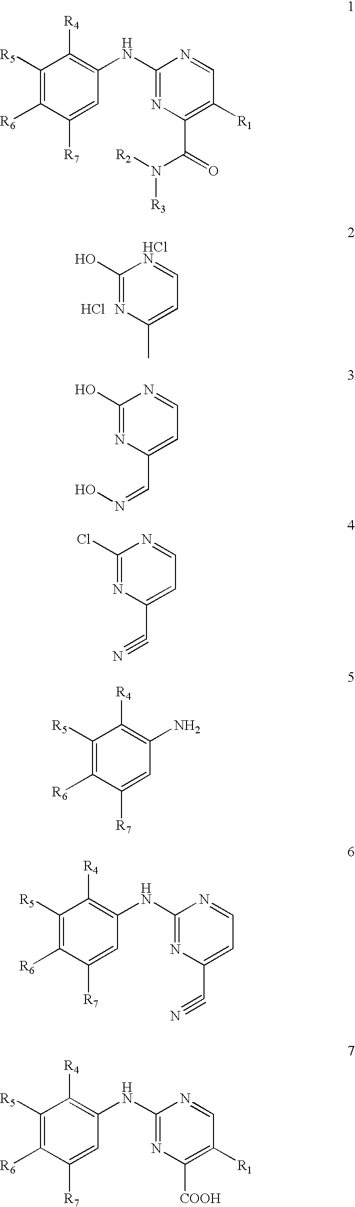

Wherein, R₁ is hydrogen; and R₂, R₃, R₄, R₅, R₆ and R₇ have the same meanings as defined above.

Further, the compound of formula 1 wherein R₁ has the same meaning as defined above in the formula 1 but not hydrogen may be prepared by a method comprising the steps of:

1) allowing a compound of formula 8, thionylchloride (SOCl₂) and methanol to react in a solvent to obtain a compound of formula 9;

2) dissolving the compound of formula 9 in a solvent, and adding an oxidizing agent thereto to obtain a compound of formula 10;

3) subjecting the compound of formula 10 and a compound of formula 5 to a reaction under microwave irradiation to obtain a compound of formula 11;

4) dissolving the compound of formula 11 in an alkali hydroxide, refluxing the mixture, and adding a strong acid thereto until pH of the mixture becomes 3, to obtain a compound of formula 7; and

5) amidating the compound of formula 7 with a compound of formula R₂R₃NH in a solvent in the presence of a coupling agent to obtain the compound of formula 1:

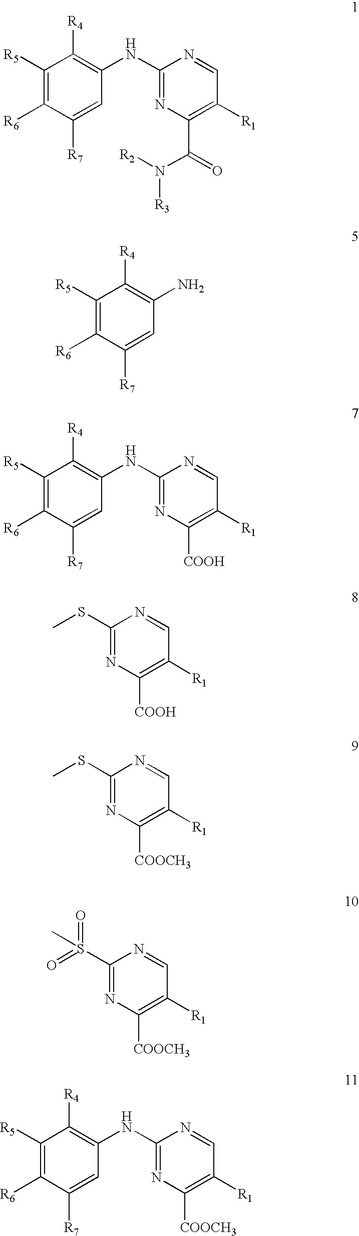

wherein, R₁ is hydroxy, halogen, C₁₋₂ alkyloxy or C₁₋₂ alkyl; R₃ is hydrogen; and R₂, R₄, R₅, R₆ and R₇ have the same meanings as defined in formula 1.

In the inventive method for preparing the compound of formula 1 wherein R₁ is hydrogen, the process for preparing the compound of formula 7 is shown in Reaction Scheme 1.

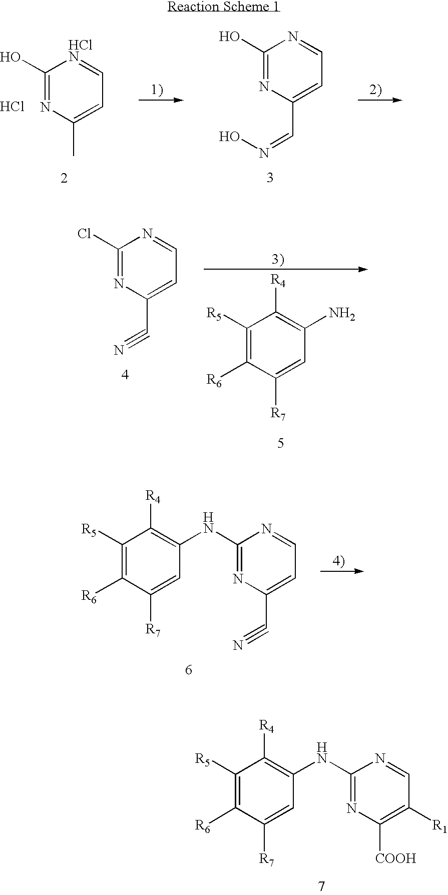

wherein,

R₁ is hydrogen; and R₄, R₅, R₆ and R₇ have the same meanings as defined above.

As shown in Reaction Scheme 1, 2-hydroxy-4-methyl pyridine (compound 2) may be first reacted with NaNO₂ in a solvent to obtain a 2-hydroxy-4-carboxyaldehyde oxime compound (compound 3). The solvent may be acetic acid, dilute hydrochloric acid or dilute sulfonic acid, and the reaction may be carried out at 0° C. to room temperature.

In step 2, the compound 3 may be reacted with a halogenating agent to obtain 2-chloropyrimidine-4-carbonitrile compound (compound 4). The halogenating agent may be POCl₃ or SOCl₂, and the solvent may be dichloromethane or methanol.

In step 3, a reaction driven by microwave irradiation of the compound 4 and an aniline compound (compound 5) in an amount ranging from 1 to 1.2 equivalents based on the compound 4 may be conducted in a solvent to obtain a 2-phenyl amino-pyrimidine-4-carbonitrile compound (compound 6). Wherein, the solvent may be 2-ethoxyethanol, dimethyl sulfoxide or DMF, the aniline compound may be preferably 3-ethoxy aniline, 3-methoxy aniline, 4-methoxy aniline, 1,3-dimethoxy aniline, 3-benzylalkyl aniline, 3-fluoro aniline, 3,4-difluoro aniline, 3,5-difluoro aniline, 2,4-difluoro aniline, 3-amido aniline, 3-nitro aniline, 4-amino ethyl aniline or 4-chloro aniline, and the microwave irradiation may be carried out with a power of 100 to 300 W, preferably about 200 W under a pressure of 0 to 150 psi, preferably about 100 psi at a temperature of 100 to 150° C., preferably about 130° C.

In step 4, the compound 6 may be dissolved in an alkali hydroxide in an amount preferably ranging from 1 to 3 equivalents based on the compound 6, refluxed, and a strong acid may be added thereto until pH of the mixture becomes 3 to obtain a compound of formula 7 (compound 7). The alkali hydroxide may be NaOH, KOH, CsOH or LiOH, and the strong acid may be HCl, H₂SO₄ or HNO₃.

In the inventive method for preparing the compound of formula 1 wherein R₁ is hydroxy, halogen, C₁₋₂ alkyloxy or C₁₋₂alkyl, a process for the preparation of the compound of formula 7 is shown in Reaction Scheme 2.

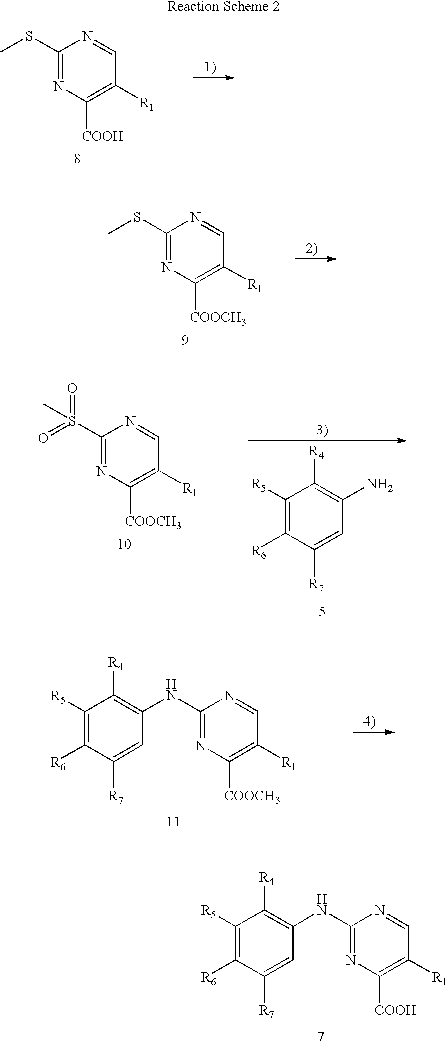

wherein, R₁ is hydroxy, halogen, C₁₋₂ alkyloxy or C₁₋₂alkyl; and R₄, R₅, R₆ and R₇ have the same meanings as defined above.

As shown in Reaction Scheme 2, 5-chloro-2-methylsulfanyl-pyrimidine-4-carboxylic acid (compound 8) may be first reacted with thionylchloride (SOCl₂) in an amount ranging from 1 to 2.1 equivalents based on the compound 8, together with methanol in a solvent to obtain a 5-chloro-2-methylsulfanyl-pyrimidine-4-carboxylic acid methyl ester compound (compound 9). The solvent used in this step may be DMF, dichloromethane or dimethylsulfoxide.

In step 2, the compound 9 may be dissolved in a solvent, an oxidizing agent in an amount ranging from 2.5 to 3 equivalents based on the compound 9 may be added thereto at 0° C., and the mixture may be reacted at room temperature for 2 hours to obtain a 5-chloro-2-methanesulfonyl-pyrimidine-4-carboxylic acid methyl ester (compound 10). The solvent may be chloroform or dichloromethane, and the oxidizing agent may be m-chloroperoxybenzoic acid, sodium methaperiodate or potassium permanganate.

In step 3, a microwave-mediated reaction of the compound 10 and an aniline compound (compound 5) in an amount ranging from 1 to 1.2 equivalents based on the compound 10 may be conducted to obtain 5-chloro-2-phenylamino-pyrimidine-4-carboxylic acid methyl ester (compound 11). The microwave irradiation may be carried out in a solvent selected from the group consisting of 2-ethoxyethanol, dimethyl sulfoxide and DMF, with a power of 100 to 300 W, preferably about 200 W under a pressure of 0 to 150 psi, preferably about 100 psi at a temperature of 100 to 150° C., preferably about 130° C.

In step 4, the compound 11 may be dissolved in an alkali hydroxide in an amount preferably ranging from 1 to 3 equivalents based on the compound 6, refluxed, and a strong acid may be added thereto until pH of the mixture becomes 3 to obtain a compound of formula 7 (compound 7). The alkali hydroxide may be NaOH, KOH, CsOH or LiOH, and the strong acid may be HCl, H₂SO₄ or HNO₃.

In the inventive method, the compounds of formula 2 and 8 used as stating materials are commercially available.

The compound of formula 1 of the present invention, as shown in Reaction Scheme 3, may be prepared by allowing an esterification of the compound of formula 7 obtained by the process described in Reaction Scheme 1 or 2.

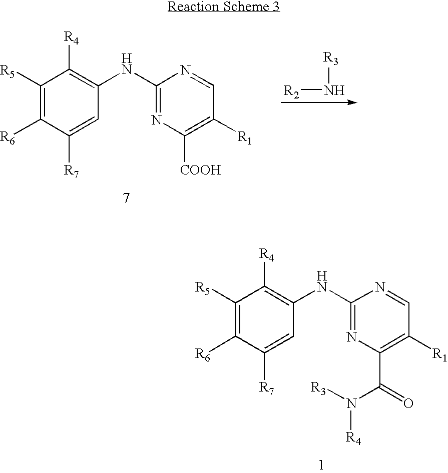

wherein, R₁, R₂, R₃, R₄, R₅, R₆ and R₇ have the same meanings as defined above.

The reaction shown in Reaction Scheme 3 may be carried out in an organic solvent including DMF using a coupling agent, such as HOBT (1-hydroxybenzotriazole/(i-Pr)₂EtN (diisopropylethyl amine) or HOBT/Et3N (triethylamine), and pyBop ((benzotriazole-1-yl-oxy)tripyrrolidinophosphonium hexafluorophosphate), HBTU (O-benzotriazole-N,N,N′,N′-tetramethyluronium haxafluorophosphate) or TBTU (O-(benzotriazole-1-yl)-N,N,N′,N′-tetramethyluronium tetrafluoroborate). The coupling agent may be employed in an amount ranging from 1.5 to 3 equivalents based on the compound of formula 7, the compound of formula R₂R₃NH may be used in an amount ranging from 1 to 2 equivalents based on the compound of formula 7, and the reaction may be conducted at room temperature to 40° C. for 30 min to 24 hours.

Further, a compound of formula 1 wherein R₅ is C₁₋₆ alkylamino C₁₋₆alkyleneamine (a compound of formula 15a) may be prepared by a method comprising the steps of:

1) reacting a compound of formula 1 wherein R₅ is NO₂ with p-chlorobenzyl chloride in a solvent to obtain a compound of formula 12a;

2) reducing of the compound of formula 12a to obtain a compound of formula 13a;

3) reacting the compound of formula 13a with aminoalkylhalide to obtain a compound of formula 14a; and

4) removing the p-chlorobenzyl group from the compound of formula 14a to obtain the compound of formula 15a:

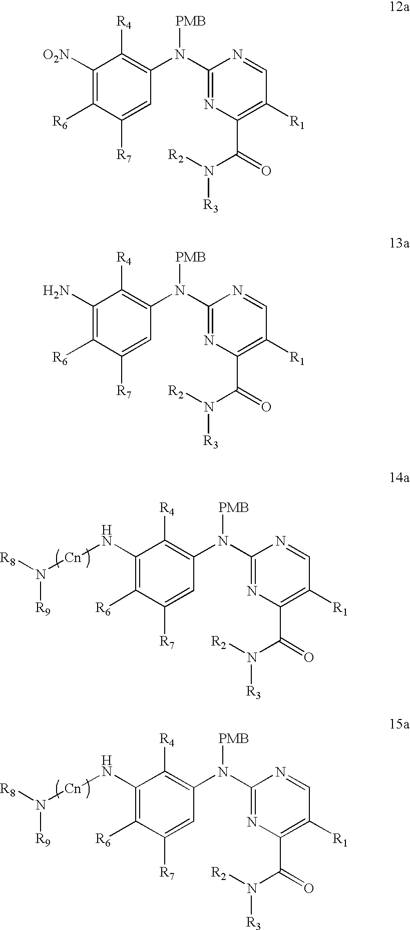

wherein, (Cn) is (CH₂)ₙ (wherein, n is an integer 0 to 3); R₂, R₃, R₄, R₆ and R₇ have the same meanings as defined above; and R₈ and R₉ are each independently methyl or ethyl, or fused together with the nitrogen to which they are attached to form a substituted ring compound.

The inventive method for preparing the compound of formula 15a are shown in Reaction Scheme 4.

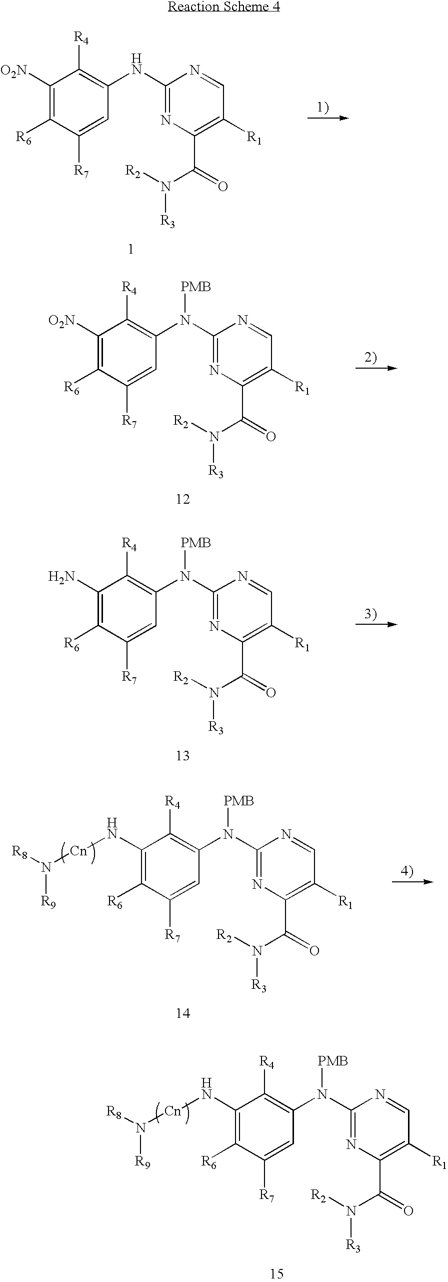

wherein, (Cn), R₂, R₃, R₄, R₆, R₇, R₈ and R₉ have the same meanings as defined above.

As shown in Reaction Scheme 4, a compound of formula 1 wherein R₅ is NO₂ (compound 1) may be first reacted with p-methoxybenzyl chloride in the presence of a base in a solvent to obtain a compound of formula 12a (compound 12). The solvent may be DMF, and the reaction may be conducted at room temperature.

In step 2, the compound 12 may be reduced in the presence of a catalyst and a hydrogen donor in a solvent to obtain a compound of formula 13a (compound 13). The catalyst may be Raney Ni, Pd/C, FeCl₂ or SnCl₂, the hydrogen donor may be H₂ or hydrazinyl hydrate, and the solvent may be methanol or dimethylformaldehyde.

In step 3, the compound 13 may be reacted with alkylaminoalkylhalide in the presence of a base in a solvent to obtain a compound of formula 14a (compound 14). The alkylaminoalkylhalide may be N-(2-chloroethyl)morpholine hydrochloride, the solvent may be 2-ethoxyethanol or dimethylformaldehyde, and the reaction may be carried out in a sealed tube at 100° C. to 200° C., preferably 110° C.

In step 4, the compound 14 may be refluxed together with a strong acid in a solvent, and the reaction mixture may be neutralized with a base to obtain a compound of formula 15a (compound 15). The strong acid may be trifluoro acetic acid or hydrochloric acid, the solvent may be methylene chloride, and the reaction may be conducted at 150° C. to 200° C.

Meanwhile, a compound of formula 1 wherein R₆ is C₁₋₆ alkylaminoC₁₋₆alkyleneamine (a compound of formula 15b) may be prepared by the method for preparing the compound of formula 15a described above, except using a compound of formula 1 wherein R₆ is NO₂ as a starting material:

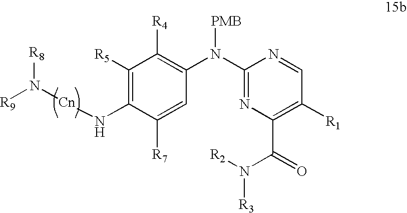

wherein, (Cn), R₂, R₃, R₄, R₅, R₇, R₈ and R₉ have the same meanings as defined above.

Meanwhile, a compound of formula 1 wherein R₁ is hydrogen, R₅ is linear or cyclic C₁₋₈ alkyl comprising one or more nitrogen, sulfur or oxygen in its chain structure (a compound of formula 23a) may be prepared by

1) reacting a compound of formula 16a with p-chlorobenzyl chloride in a solvent to obtain a compound of formula 17a;

2) reducing the compound of formula 17a to obtain a compound of formula 18a;

3) subjecting the compound of formula 18a and a compound of formula 4 to a reaction under microwave irradiation to obtain a compound of formula 19a;

4) dissolving the compound of formula 19a in an alkali hydroxide, refluxing the mixture, and adding a strong acid thereto until pH of the mixture becomes 3 to obtain a compound of formula 20a;

5) amidating the compound of formula 20a with a compound of formula R₂R₃NH in a organic solvent in the presence of a coupling agent to obtain a compound of formula 21a;

6) removing the p-chlorobenzyl group from the compound of formula 21a to obtain a compound of formula 22a; and

7) alkylating the compound of formula 22a in the presence of a base to obtain a compound of formula 23a:

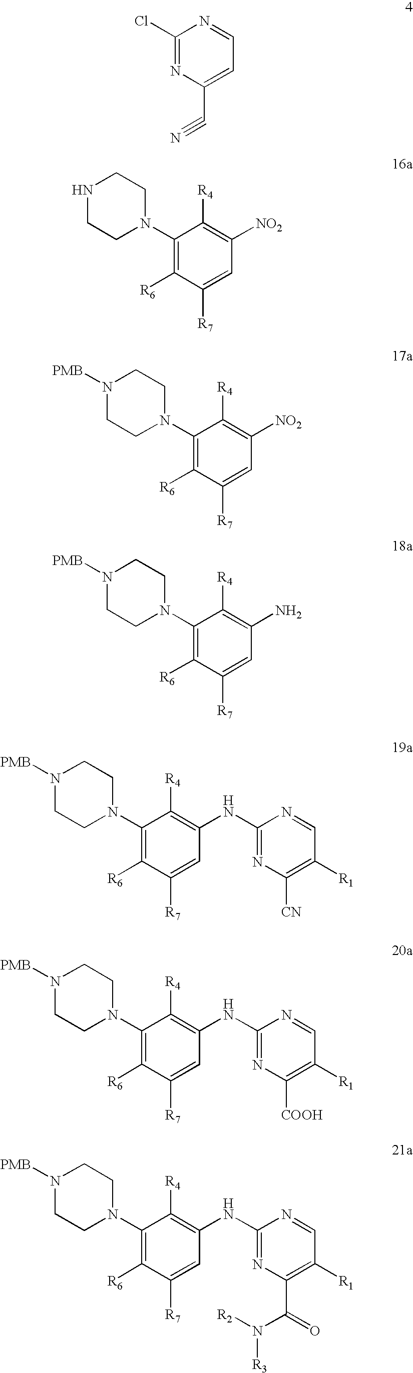

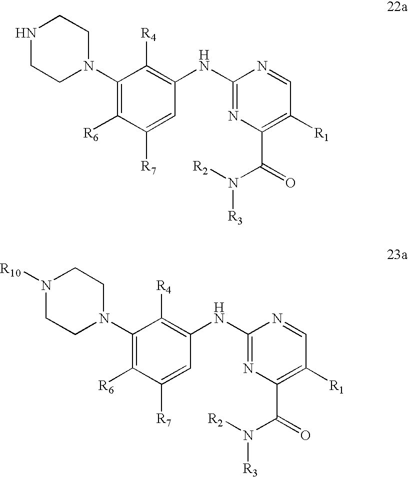

wherein, R₁ is H; R₂, R₃, R₄, R₆ and R₇ have the same meanings defined above; and R₁₀ is C₁₋₆ alkyl.

The inventive method for preparing the compound of formula 23a is shown in Reaction Scheme 5.

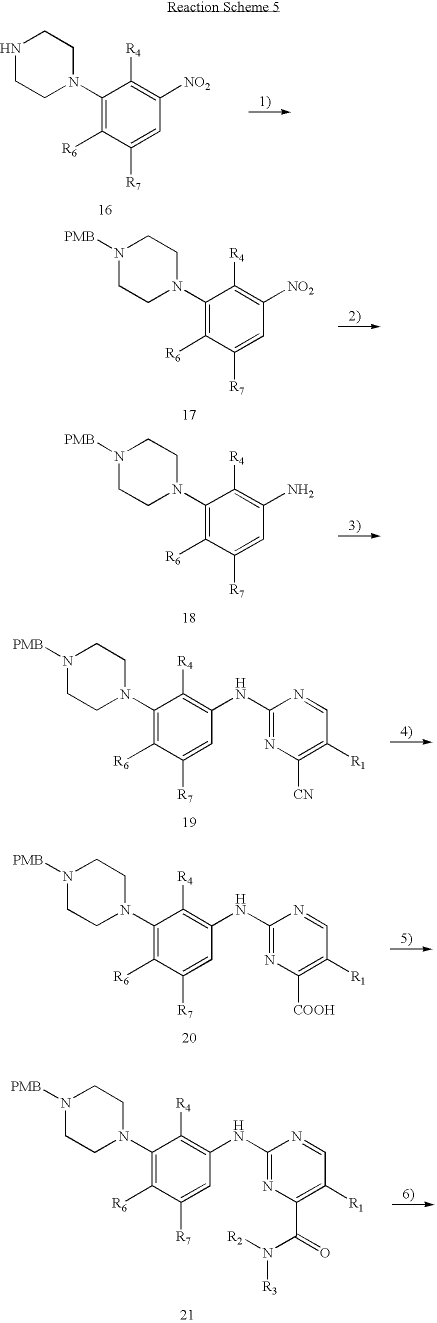

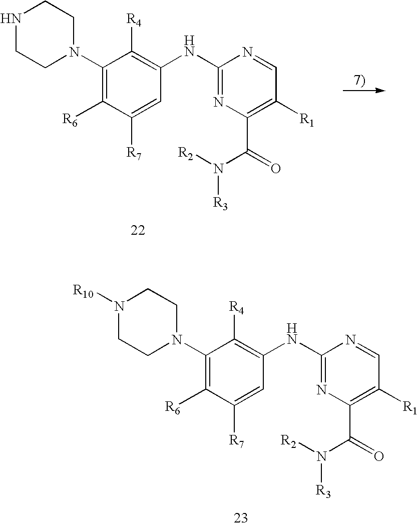

wherein, R₁ is H; R₂, R₃, R₄, R₆, R₇ and R₁₀ have the same meanings defined above.

As shown in Reaction Scheme 5, the compound of formula 16a (compound 16) may be first reacted with p-chlorobenzyl chloride in the presence of a base in a solvent to obtain a compound of formula 17a (compound 17). The compound of formula 16a used as a starting material may be prepared by a conventional method, or commercially available, while the solvent may be dimethyl formaldehyde, and the base may be sodium hydride.

In step 2, the compound 17 may be reduced in the presence of an catalyst and a hydrogen donor in a solvent to obtain a compound of formula 18a (compound 18). The catalyst may be Raney Ni, Pd/C, FeCl₂ or SnCl₂, the hydrogen donor may be H₂ or hydrazinyl hydrate, and the solvent may be ethanol, methanol or dimethylformaldehyde.

In step 3, a microwave-mediated reaction of the compound of formula 4 and the compound 18 in an amount ranging from 1 to 1.2 equivalents based on the compound of formula 4 may be conducted to obtained the compound of formula 19a (compound 19). The organic solvent may be 2-ethoxyethanol, dimethyl sulfoxide or DMF, and the microwave irradiation may be carried out with a power of 100 to 300 W, preferably about 200 W, under a pressure of 0 to 150 psi, preferably about 100 psi, at a temperature of 100 to 150° C., preferably about 130° C.

In step 4, the compound 19 may be dissolved with an alkali hydroxide in an amount ranging from 1 to 3 equivalents based on the compound 19, refluxed, and a strong acid may be added thereto until pH of the reaction mixture becomes 3 to obtain a compound of formula 20a (compound 20). The alkali hydroxide may be NaOH, KOH, CsOH or LiOH, and the strong acid may be HCl, H₂SO₄ or HNO₃.

In step 5, amidation of the compound 20 may be allowed with a compound of formula R₂R₃NH in the presence of a coupling agent in an organic solvent to obtain a compound of formula 21a (compound 21). The organic solvent may be DMF, the coupling agent may be HOBT (1-hydroxybenzotriazole/(i-Pr)₂EtN (diisopropylethyl amine) or HOBT/Et3N (triethylamine), and pyBop ((benzotriazole-1-yl-alkyl)tripyrrolidinophosphonium hexafluorophosphate), HBTU (O-benzotriazole-N,N,N′,N′-tetramethyluronium hexafluorophosphate) or TBTU (O-(benzotriazole-1-yl)-N,N,N′,N′-tetramethyluronium tetrafluoroborate). Further, the coupling agent may be employed in an amount ranging from 1.5 to 3 equivalents based on the compound 20, the compound of formula R₂R₃NH may be employed in an amount ranging from 1 to 2 equivalents based on the compound 20, and the reaction may be conducted at room temperature to 40° C. for 30 min to 24 hours.

In step 6, the compound 21 may be refluxed in the presence of a strong acid in a solvent, and the reaction mixture may be neutralized with a base to obtain a compound of formula 22a (compound 22). The solvent may be methylene chloride, the base may be sodiumbicarbonate, the strong acid may be trifluoroacetic acid or hydrochloric acid, and the reaction may be conducted at 150° C. to 200° C.

In step 7, the compound 22 may be reacted with alkyliodide in a solvent in the presence of a base to obtain a compound of formula 23a (compound 23). The base may be sodiumbicarbonate, and the solvent may be dimethyl formaldehyde. Also, the reaction may be carried out at room temperature to 40° C.

Meanwhile, a compound of formula 1 wherein R₆ is C₁₋₈ alkyl or C₃₋₈ cycloalkyl comprising one or more nitrogen, sulfur or oxygen (a compound of formula 23b) may be prepared by the method for preparing the compound of formula 23a described above, except for using a compound of formula 16b as a starting material:

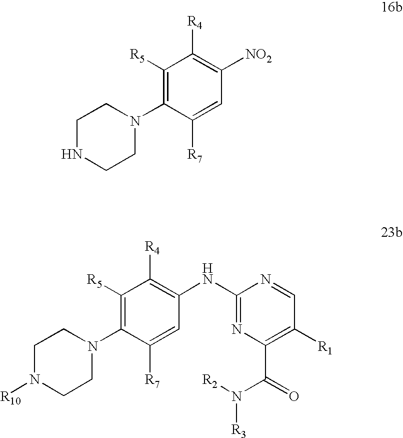

wherein, R₁ is H; R₂, R₃, R₄, R₅ and R₇ have the same meanings as defined above; and R₁₀ is C₁₋₆ alkyl.

In accordance with further aspect of the present invention, there is provided a composition for inhibition of the protein kinase activity comprising said aminopyrimidine derivatives, or a pharmaceutically acceptable salt, hydrate, solvate or isomer thereof as an active ingredient.

The protein kinases may be selected from the group consisting of glycogen synthase kinase 3 (GSK), aurora kinase, extracellular signal-regulated kinase (ERK), protein kinase B (AKT), cyclin-dependent kinase (CDK), p38 (protein 38) mitogen-activated protein kinase (MAPK) and c-Jun N-terminal kinase (JNK), preferably aurora kinase, GSK, JNK, CDK and p38 MAPK.

In addition, the inventive aminopyrimidine derivative of formula 1, or a pharmaceutically acceptable salt, hydrate, solvate or isomer thereof as an active ingredient may be used in an anticancer composition since it can efficiently inhibit the activities of several protein kinases including aurora kinase to repress the proliferation of cancer cells. Accordingly, in the present invention, there is provided a pharmaceutical composition comprising said aminopyrimidine derivative, or a pharmaceutically acceptable salt, hydrate, solvate or isomer thereof as an active ingredient.

The salt, hydrate, solvate or isomer of the compound of formula 1 may be prepared from the compound of formula 1 in accordance with the conventional method.

The pharmaceutically acceptable composition may be formulated for oral or parenteral administration. The composition for oral administration may take various forms such as tablets, powder, rigid or soft gelatin capsules, solution, dispersion, emulsions, syrups and granules, such formulations may comprise the active ingredient together with diluting agents (e.g., lactose, dextrose, sucrose, mannitol, sorbitol, cellulose and/or glycine), and lubricants (e.g., silica, talc, stearic acid and a magnesium or calcium salt thereof and/or polyethyleneglycol). Further, these tablets may comprise binding agents such as magnesium aluminium silicate, starch paste, gelatin, tragacanth, methylcellulose, sodium carboxymethylcellulose and/or polyvinylpyrrolidine, and may further comprise disintergrants such as starch, agarose, alginate or a sodium salt thereof or an effervescent mixture and/or an absorbing, colouring, flavouring, and sweetening agents.

Further, the inventive pharmaceutical composition may take forms of preferably injections further comprising saline solution or suspensions when formulated for parenteral administration.

The pharmaceutical composition may be sterilized and/or may further comprise preservatives, stabilizing agents, hydrating agents or emulsifiers, salts for controlling osmotic pressure and/or supplementary agents including buffer agents and other therapeutically available materials, and may be prepared by the conventional mixing, granulating or coating methods.

A proposed daily dose of the compound of formula 1 used as an active ingredient in the inventive composition for administration to a mammal including human is about from 2.5 mg/kg weight to 100 mg/kg weight, more preferably about from 5 mg/kg weight to 60 mg/kg weight. It should be understood that the daily dose should be determined in light of various relevant factors including the condition to be treated, the severity of the patient's symptoms, the route of administration, or the physiological form of the anticancer agent; and, therefore, the dosage suggested above should not be construed to limit the scope of the invention in anyway.

The following Examples are intended to further illustrate the present invention without limiting its scope.

### Preparation Example 1

### Preparation of 2-chloro pyrimidine-4-carbonitrile

### Step 1) Preparation of 2-hydroxy-4-carboxyaldehyde oxime

2-hydroxy-4-methylpyrimidine (2 g) and sodium nitrate (1.8 g, 27.2 mmol 1.5 equivalent) were slowly added to 20 ml of 50% acetic acid at 0° C., and the mixture was stirred at room temperature for 3 hours. The resulting mixture was filtered, washed with water and dried to obtain the light yellow title compound (2.2 g; yield: 89%).

### Step 2) Preparation of 2-chloro pyrimidine-4-carbonitrile

The compound obtained in Step 1 (4 g, 28.7 mmol) was added to 10 ml of trichlorophosphrous, and the mixture was stirred for 4 hours. The resulting mixture was concentrated under a reduced pressure to remove the solvent, and sequentially washed with sodium bicarbonate solution (200 ml×2) and salt solution. The resulting residue was subjected to silica gel column chromatography (eluent: n-hexane/ethyl acetate=4:1) to obtain the title compound (2.4 g; yield: 60%).

### Preparation Example 2

### Preparation of 5-chloro-2-methanesulfonyl-pyrimidine-4-carboxylic acid methyl ester

### Step 1) Preparation of 5-chloro-2-methylsulfanyl-pyrimidine-4-carboxylic acid methyl ester

5-chloro-2-methylsulfanyl-pyrimidine-4-carboxylic acid (2.04 g, 10 mmol) was added to methanol, and 10 ml of thionyl chloride was slowly added thereto, followed by stirring the mixture for 5 hours. The resulting mixture was concentrated under a reduced pressure to remove the solvent, and sequentially washed with sodium bicarbonate solution and salt solution. The resulting residue was subjected to silica gel column chromatography (eluent: n-hexane/ethyl acetate=5:1) to obtain the title compound (2.1 g; yield: 95%).

### Step 2) Preparation of 5-chloro-2-methanesulfonyl-pyrimidine-4-carboxylic acid methyl ester

The compound obtained in Step 1 (2.1 g, 9.6 mmol) was added to 100 ml of methylene chloride, and m-chloroperalkylbenzoic acid (32 g, 19 mmol) was slowly added thereto, followed by stirring the mixture at room temperature for 5 hours. The resulting mixture was concentrated under a reduced pressure to remove the solvent, and sequentially washed with sodium bicarbonate and salt solution. The resulting residue was subjected to silica gel column chromatography (eluent: n-hexane/ethyl acetate=5:1) to obtain the title compound (1.9 g; yield: 90%).

### Example 1

### Preparation of 2-(3-ethoxy-phenylamino)-pyrimidine-4-carboxylic acid cyclohexylamide

### Step 1) Preparation of 2-(3-ethoxy-phenylamino)-pyrimidine-4-carbonitrile

2-chloro pyrimidine-4-carbonitrile compound obtained in Preparation Example 1 (418 mg, 3.02 mmol) was subjected to a reaction under a microwave irradiation (200 W, 100 psi, 130° C.) with 3-ethoxyaniline (622 mg, 4.54 mmol) for 30 min to obtain the title compound (514 mg; yield: 71%).

### Step 2) Preparation of 2-(3-ethoxy-phenylamino)-phenyl amino-pyrimidine-4-carboxylic acid

The compound obtained in Step 1 (514 mg, 2.14 mmol) was dissolved in 2 ml of 3 N NaOH, and the mixture was refluxed for 3 hours. pH of the resulting solution was adjusted to 3 by adding 3 N HCl to obtain the title compound (484 mg; yield: 87%).

### Step 3) Preparation of 2-(3-ethoxy-phenylamino)-pyrimidine-4-carboxylic acid cyclohexylamide

The compound obtained in Step 2 (49 mg, 0.19 mmol) was dissolved in 2 ml of DMF, and HOBt (37 mg, 0.28 mmol), (i-Pr)₂EtN (65 mg, 0.50 mmol) and EDC (53 mg, 0.28 mmol) were added thereto. Cyclohexylamine (27.7 mg, 0.28 mmol) was added to the mixture, and the resulting mixture was stirred at room temperature for 1 day, followed by removing the solvent under a reduced pressure. The resulting residue was extracted with methylene chloride, dried over MgSO₄, concentrated under a reduced pressure, and subjected to silica gel column chromatography (eluent: methylene chloride/methanol=10:1) to obtain the title compound (57 mg; yield: 63%).

The procedure of Example 1 was repeated except for using each compounds corresponded to the compounds of formula 7 and aniline compound to prepare following compounds of Examples 1 to 23, 35, 37 to 41, 43 to 45, 58 to 77, 83 to 85, 92, 93, 100, 101, 111 to 114, and 119 listed in Table 1:

Example 2: 2-phenylamino-pyrimidine-4-carboxylic acid[3-(4,5-dichloro-imidazol-1-yl)-propyl]-amide;
Example 3: 2-(3-fluoro-phenylamino)-pyrimidine-4-carboxylic acid[2-(4-ethylsulfonylamino-phenyl)-ethyl]-amide;
Example 4: 2-(4-fluoro-phenylamino)-pyrimidine-4-carboxylic acid[2-(4-ethylsulfonylamino-phenyl)-ethyl]-amide;
Example 5: 2-(2,4-difluoro-phenylamino)-pyrimidine-4-carboxylic acid[2-(4-ethylsulfonylamino-phenyl)-ethyl]-amide;
Example 6: 2-(4-chloro-phenylamino)-pyrimidine-4-carboxylic acid[2-(4-ethylsulfonylamino-phenyl)-ethyl]-amide;
Example 7: 2-(3,4-difluoro-phenylamino)-pyrimidine-4-carboxylic acid[2-(4-ethylsulfonylamino-phenyl)-ethyl]-amide;
Example 8: 2-(3,5-difluoro-phenylamino)-pyrimidine-4-carboxylic acid[2-(4-ethylsulfonylamino-phenyl)-ethyl]-amide
Example 9: 2-[4-(2-amino-ethyl)-phenylamino]-pyrimidine-4-carboxylic acid[2-(4-ethylsulfonylamino-phenyl)-ethyl]-amide;
Example 10: 2-(3-methoxy-phenylamino)-pyrimidine-4-carboxylic acid (2-pyridin-3-yl-ethyl)-amide;
Example 11: 2-(4-methoxy-phenylamino)-pyrimidine-4-carboxylic acid (2-pyridin-4-yl-ethyl)-amide;
Example 12: 2-(4-methoxy-phenylamino)-pyrimidine-4-carboxylic acid (2-pyridin-3-yl-ethyl)-amide;
Example 13: 2-(4-methoxy-phenylamino)-pyrimidine-4-carboxylic acid[2-(2-hydroxy-phenyl)-ethyl]-amide;
Example 14: 2-(4-methoxy-phenylamino)-pyrimidine-4-carboxylic acid[2-(3-hydroxy-phenyl)-ethyl]-amide;
Example 15: 2-(4-methoxy-phenylamino)-pyrimidine-4-carboxylic acid (2-{4-[2-(4-ethyl-piperazin-1-yl)-acetylamino]-phenyl}-ethyl)-amide;
Example 16: 2-(4-methoxy-phenylamino)-pyrimidine-4-carboxylic acid[2-(4-acetylamino-phenyl)-ethyl]-amide;
Example 17: 2-(4-methoxy-phenylamino)-pyrimidine-4-carboxylic acid[2-(4-morpholin-4-yl-phenyl)-ethyl]-amide;
Example 18: 2-(4-methoxy-phenylamino)-pyrimidine-4-carboxylic acid{2-[4-(2-dimethylamino-acetylamino)-phenyl]-ethyl}-amide;
Example 19: 2-(4-methoxy-phenylamino)-pyrimidine-4-carboxylic acid[3-(2-methyl-imidazol-1-yl)-propyl]-amide;
Example 20: 2-(3,5-difluoro-phenylamino)-N-(2-(pyridin-4-yl)ethyl)pyrimidine-4-carboxyamide;
Example 21: 2-(4-hydroxy-phenylamino)-N-(2-(pyridin-2-yl)ethyl)pyrimidine-4-carboxyamide;
Example 22: 2-(4-hydroxy-phenylamino)-pyrimidine-4-carboxylic acid (2-pyridin-3-yl-ethyl)-amide;
Example 23: 2-(3,5-difluoro-phenylamino)-N-(3-fluoro-4-hydroxy)pyrimidine-4-carboxyamide;
Example 35: 2-phenylamino-pyrimidine-4-carboxylic acid[2-(4-ethylsulfonylamino-phenyl)-ethyl]-amide;
Example 37: 2-(3-ethoxy-phenylamino)-pyrimidine-4-carboxylic acid[3-(4,5-dichloro-imidazol-1-yl)-propyl]-amide;
Example 38: 2-(3-benzylalkyl-phenylamino)-pyrimidine-4-carboxylic acid cyclohexyloamide;
Example 39: 2-(3-benzylalkyl-phenylamino)-pyrimidine-4-carboxylic acid[3-(4,5-dichloro-imidazol-1-yl)-propyl]-amide;
Example 40: 2-(3-ethoxy-phenylamino)-pyrimidine-4-carboxylic acid (4-aminocyclohexyl)-amide;
Example 41: 2-(3-ethoxy-phenylamino)-pyrimidine-4-carboxylic acid[4-(cyclopropanecarbonyl-amino)-cyclohexyl]-amide;
Example 43: 2-[4-methoxy-3-(2-morpholin-4-yl-ethylamino)-phenylamino]-pyrimidine-4-carboxylic acid cyclohexylamide;
Example 44: 2-(3-acetylamino-phenylamino)-pyrimidine-4-carboxylic acid cycloamide;
Example 45: 2-(3,5-dimethoxy-phenylamino)-pyrimidine-4-carboxylic acid cycloamide;
Example 58: 2-(3-ethoxy-phenylamino)-pyrimidine-4-carboxylic acid (2,6-dimethyl-phenyl)-amide;
Example 59: 2-(3-ethoxy-phenylamino)-pyrimidine-4-carboxylic acid[3-(cyclopropane carbonyl-amino)-cyclohexyl]-amide;
Example 60: 2-(3-ethoxy-phenylamino)-pyrimidine-4-carboxylic acid (3-acetylamino-cyclohexyl)-amide;
Example 61: 2-(3-ethoxy-phenylamino)-pyrimidine-4-carboxylic acid{3-[(thiophene-3-carbonyl)-amino]-cyclohexyl}-amide;
Example 62: 2-(3-methoxy-phenylamino)-pyrimidine-4-carboxylic acid benzyl-ethyl-amide;
Example 63: 2-(3-methoxy-phenylamino)-pyrimidine-4-carboxylic acid (2-amino-cyclohexyl)-amide;
Example 64: 2-(3-ethoxy-phenylamino)-pyrimidine-4-carboxylic acid[2-(cyclopropanecarbonyl-amino)-cyclohexyl]-amide;
Example 65: 2-(3-methoxy-phenylamino)-pyrimidine-4-carboxylic acid (2-butylamino-cyclohexyl)-amide;
Example 66: 2-(3-methoxy-phenylamino)-pyrimidine-4-carboxylic acid[2-(2-chloro-acetylamino)-cyclohexyl]-amide;
Example 67: 2-(3-methoxy-phenylamino)-pyrimidine-4-carboxylic acid (2-propylamino-cyclohexyl)-amide;
Example 68: 2-(3-methoxy-phenylamino)-pyrimidine-4-carboxylic acid (4,7,7-trimethyl-bicyclo[2.2.1]heptan-2-yl)-amide;
Example 69: 2-(3-methoxy-phenylamino)-pyrimidine-4-carboxylic acid biphenyl-2-yl amide;
Example 70: 2-(3-methoxy-phenylamino)-pyrimidine-4-carboxylic acid (2-dipropylamino-cyclohexyl)-amide;
Example 71: 2-(3-methoxy-phenylamino)-pyrimidine-4-carboxylic acid (2-ethyl-6-methyl-phenyl)-amide;
Example 72: 2-(3-methoxy-phenylamino)-pyrimidine-4-carboxylic acid aryl-phenyl-amide;
Example 73: 2-(3-methoxy-phenylamino)-pyrimidine-4-carboxylic acid cyclohexyl-ethyl-amide;
Example 74: 2-(3-methoxy-phenylamino)-pyrimidine-4-carboxylic acid (2-amino-cyclohexyl)-amide;
Example 75: 2-(3-methoxy-phenylamino)-pyrimidine-4-carboxylic acid[2-(cyclopropanecarbonyl-amino)-cyclohexyl]-amide;
Example 76: 2-(3-fluoro-phenylamino)-pyrimidine-4-carboxylic acid cyclohexyl amide;
Example 77: 2-(3-amino-phenylamino)-pyrimidine-4-carboxylic acid cyclohexylamide;
Example 83: 2-(4-morpholinophenylamino)-N-(1,7,7-trimethylbicyclo[2.2.1]heptan-2-yl)pyrimidine-4-carboxyamide;
Example 84: 2-(4-aminophenylamino)-N-(1,7,7-trimethylbicyclo[2.2.1]heptan-2-yl)pyrimidine-4-carboxyamide;
Example 85: 2-(4-(piperidin-1-yl)phenylamino)-N-(1,7,7-trimethylbicyclo[2.2.1]heptan-2-yl)pyrimidine-4-carboxyamide;
Example 92: 2-(4-(dimethylamino)ethylamino)phenylamino)-N-(1,7,7-trimethylbicyclo[2.2.1]heptan-2-yl)pyrimidine-4-carboxyamide;
Example 93: 2-(3-(dimethylamino)ethylamino)phenylamino)-N-(1,7,7-trimethylbicyclo[2.2.1]heptan-2-yl)pyrimidine-4-carboxyamide;
Example 100: 2-(3-sulfamoylphenylamino)-N-(1,7,7-trimethylbicyclo[2.2.1]heptan-2-yl)pyrimidine-4-carboxyamide;
Example 101: 2-(4-sulfamoylphenylamino)-N-(1,7,7-trimethylbicyclo[2.2.1]heptan-2-yl)pyrimidine-4-carboxyamide;
Example 111: 2-(4-(diethylamino)phenylamino)-N-(1,7,7-trimethylbicyclo[2.2.1]heptan-2-yl)pyrimidine-4-carboxyamide;
Example 112: 2-(2-(2-morpholinoethoxy)phenylamino)-N-(1,7,7-trimethylbicyclo[2.2.1]heptan-2-yl)pyrimidine-4-carboxyamide;
Example 113: 2-(2-(2-(pyrrolidin-1-yl)ethoxy)phenylamino)-N-(1,7,7-trimethylbicyclo[2.2.1]heptan-2-yl)pyrimidine-4-carboxyamide;
Example 114: 2-(3-(morpholine-4-carbonyl)phenylamino)pyrimidin-4-yl)(morpholino)methanone; and
Example 119: 2-(5-methoxy-2-(2-morpholinoethoxy)phenylamino)-N-(1,7,7-trimethylbicyclo[2.2.1]heptan-2-yl)pyrimidine-4-carboxyamide.

### Example 29

### Preparation of 5-chloro-2-phenylamino-pyrimidine-4-carboxylic acid [3-(4,5-dichloro-imidazol-1-yl)-propyl]-amide

### Step 1) Preparation of 5-chloro-2-phenylamino-pyrimidine-4-carboxylic acid methyl ester

The compound obtained in Preparation Example 2 (1.9 g, 7.6 mmol) was subjected to a reaction under a microwave irradiation (200 W, 100 psi, 130° C.) with aniline (1.06 g, 11.4 mmol) for 30 min to obtain the title compound (1.57 g; yield: 79%).

### Step 2) Preparation of 5-chloro-2-phenylamino-pyrimidine-4-carboxylic acid

The compound obtained in Step 1 (1.57 g) was added to 20 ml of 1 N NaOH, and the mixture was refluxed. After completing the reaction, pH of the resulting mixture was adjusted to pH 3 by adding 3 N HCl to obtain the title compound (1.09 g; yield: 74%).

### Step 3) Preparation of 5-chloro-2-phenylamino-pyrimidine-4-carboxylic acid [3-(4,5-dichloro-imidazol-1-yl)-propyl]-amide

The compound obtained in Step 2 (57.3 mg, 0.23 mmol) was dissolved in 2 ml of DMF, and HOBt (37 mg, 0.28 mmol), EtN (65 mg, 0.50 mmol) and EDC (53 mg, 0.28 mmol) were added thereto. 4,5-dichloro-imidazole-N-propyl amine (54 mg, 0.28 mmol) was added to the mixture, and the resulting mixture was stirred at room temperature for 1 day, followed by removing the solvent under a reduced pressure. The resulting residue was extracted with methylene chloride, dried over MgSO₄, concentrated under a reduced pressure, and subjected to silica gel column chromatography (eluent: methylene chloride/methanol=10:1) to obtain the title compound (59 mg; yield: 60%).

The procedure of Example 29 was repeated except for using each compounds corresponded to the compounds of formula 7 and aniline compound to prepare following compounds of Examples 24 to 34, 36, 42, 46 to 57, and 78 listed in Table 1:

Example 24: methyl-5-chloro-2-(3-fluorophenylamino)pyrimidine-4-carboxylate;
Example 25: 2-(benzo[1,3]dioxol-5-ylamino)-5-chloro-pyrimidine-4-carboxylic acid(3-phenyl-propyl)-amide;
Example 26: 5-chloro-2-phenylamino-pyrimidine-4-carboxylic acid(3-phenyl-propyl)-amide;
Example 27: 2-(benzole[1,3]dioxol-5-ylamino)-5-chloro-pyrimidine-4-carboxylic acid[3-(4,5-dichloro-imidazol-1-yl)-propyl]-amide;
Example 28: 5-chloro-2-(3-methoxy-phenylamino)-pyrimidine-4-carboxylic acid[3-(4,5-dichloro-imidazol-1-yl)-propyl]-amide;
Example 30: 2-(benzo[d][1,3]dioxalyl-5-amino)-5-chloro-N-methylpyrimidine-4-carboxyamide;
Example 31: 5-chloroN-(3-(4,5-dichloro-1H-imidazolyl-1-yl)propyl)-2-(3-methoxyphenylamino)pyrimidine-4-carboxyamide;
Example 32: 5-chloro-2-phenylamino-pyrimidine-4-carboxylic acid cyclohexylamide;
Example 33: 2-(benzo[1,3]dioxol-5-ylamino)-5-chloro-pyrimidine-4-carboxylic acid cyclohexylamide;
Example 34; 5-chloro-2-(3-ethoxy-phenylamino)-pyrimidine-4-carboxylic acid cycloamide;
Example 36; 5-chloro-2-(3-methoxy-phenylamino)-pyrimidine-4-carboxylic acid (4-amino-cyclohexyl)-amide;
Example 42: 5-chloro-2-(3-methoxy-phenylamino)-pyrimidine-4-carboxylic acid[4-(2,2-dimethyl-propionylamino)-cyclohexyl]-amide;
Example 46: 5-chloro-2-(3-methoxy-phenylamino)-pyrimidine-4-carboxylic acid (3-pyrrolidin-1-yl-propyl)-amide;
Example 47: 5-chloro-2-(3-methoxy-phenylamino)-pyrimidine-4-carboxylic acid[3-(4-methyl-piperazin-1-yl)-propyl]-amide;
Example 48: 5-chloro-2-(3-methoxy-phenylamino)-pyrimidine-4-carboxylic acid (1-methyl-1-phenyl-ethyl)-amide;
Example 49: 5-chloro-2-(3-methoxy-phenylamino)-pyrimidine-4-carboxylic acid[4-(propane-2-sulfonylamino)-cyclohexyl]-amide;
Example 50: 2-(3-ethoxy-phenylamino)-pyrimidine-4-carboxylic acid(3-aminocyclohexyl)-amide;
Example 51: 5-chloro-2-(3-methoxy-phenylamino)-pyrimidine-4-carboxylic acid (4-tert-butyl-cyclohexyl)-amide;
Example 52: 5-chloro-2-(3-methoxy-phenylamino)-pyrimidine-4-carboxylic acid cycloheptylamide;
Example 53: 5-chloro-2-(3-methoxy-phenylamino)-pyrimidine-4-carboxylic acid (2,2,6,6-tetramethyl-piperidin-4-yl)-amide;
Example 54: 5-chloro-2-(3-methoxy-phenylamino)-pyrimidine-4-carboxylic acid inden-2-yl amide;
Example 55: 5-chloro-2-(3-methoxy-phenylamino)-pyrimidine-4-carboxylic acid (1,2,3,4-tetrahydro-naphthalen-1-yl)-amide;
Example 56: 5-chloro-2-(3-methoxy-phenylamino)-pyrimidine-4-carboxylic acid[1-(4-chloro-phenyl)-propyl]-amide;
Example 57: 5-chloro-2-(3-methoxy-phenylamino)-pyrimidine-4-carboxylic acid sec-butylamide; and
Example 78: 2-(3-amino-phenylamino)-pyrimidine-4-carboxylic acid (1,7,7-trimethyl-bicyclo[2.2.1]hepten-2-yl)-amide.

### Example 97

### Preparation of 2-(3-(3-morpholinopropylamino)phenyl amino)-N-1-(1,7,7-trimethoxybicyclo[2.2.1]heptan-2-yl)pyrimidine-4-carboxyamide

### Step 1) Preparation of 2-(4-methoxybenzyl)-3-nitrophenyl amino)-N-1-(1,7,7-trimethoxybicyclo[2.2.1]heptan-2-yl)pyrimidine chloro-4-carboxyamide

2-(-3-nitrophenyl amino)-N-1-(1,7,7-trimethoxybicyclo[2.2.1]heptan-2-yl)pyrimidine chloro-4-carboxyamide (4.513 g, 11.4 mmol) was dissolved in 10 ml of DMF, and NaH (686 mg, 17 mmol, 1.2 equivalent) was slowly added thereto at 0° C., followed by reacting the mixture for 10 min. 2.05 ml of p-methoxybenzyl chloride dissolved in 10 ml of DMF was slowly added thereto, and the mixture was reacted at a room temperature for 1 hour, concentrated under a reduced pressure to remove the solvent, and sequentially washed with sodium bicarbonate (200 ml×2) and salt solution. The resulting residue was subjected to silica gel column chromatography (eluent: n-hexane/ethyl acetate=3:1) to obtain the title compound (5.8 g; yield: 99%).

### Step 2) Preparation of 2-((3-aminomethyl)-4-methoxybenzyl amino)-N-1-(1,7,7-trimethoxybicyclo[2.2.1]heptan-2-yl)pyrimidine-4-carboxyamide

70 ml of EtOH was added to the compound obtained in Step 1 (6.2 g), 7 ml of Raney Ni and 2 ml of NH₂NH₂ were added thereto, followed by reacting the mixture at a room temperature for 4 hours with an empty balloon put. The resulting mixture was filtered with celite to remove Ni and concentrated under a reduced pressure to remove the solvent. The resulting residue was subjected to silica gel column chromatography (eluent: n-hexane/ethyl acetate=3:1) to obtain the title compound (4.43 g; yield: 80%).

### Step 3) Preparation of 2-((4-methoxybenzyl)-(3-(3-morpholinopropyl)phenyl)amino)-N-1-(1,7,7-trimethoxybicyclo[2.2.1]heptan-2-yl)pyrimidine-4-carboxyamide

The compound obtained in Step 2 (2 g), N-(2-chloroethyl)morpholine hydrochloride (1.53 g), potassium iodide (684 mg) and potassium bicarbonate (1.14 g) were placed in a sealed tube, and 40 ml of 2-ethoxy ethanol was added thereto, followed by reacting the mixture at 110° C. for 2.5 hours. After completing the reaction by adding water, the resulting mixture was extracted with methylene chloride and concentrated under a reduced pressure to remove the solvent. The resulting residue was subjected to silica gel column chromatography (eluent: methylene chloride containing a small amount of Et₃N/methanol=40:1 mixture) to obtain the title compound (900 mg; yield: 36%).

### Step 4) Preparation of 2-(3-(3-morpholinopropylamino)phenylamino)-N-1-(1,7,7-trimethoxybicyclo[2.2.1]heptan-2-yl)pyrimidine-4-carboxyamide

The compound obtained in Step 3 (900 mg) was dissolved in a small amount of methylene chloride, and 40 ml of trifluoroacetic acid was added thereto, followed by refluxing the mixture for 6 hours and neutralizing the mixture with sodium bicarbonate aqueous solution. The resulting mixture was extracted with methylene chloride and concentrated under a reduced pressure to remove the solvent. The resulting residue was subjected to silica gel column chromatography (eluent: methylene chloride containing a small amount of Et₃N/ethylacetate/methanol=30:10:1 mixture) to obtain the title compound (94 mg; yield: 83%).

The procedure of Example 97 was repeated except for using each compounds corresponded to the compounds of formula 12a or 12b as a starting material to prepare following compounds of Examples 79 to 82, 86 to 91, 94 to 99, 102, 106, 107, 117 and 118 listed in Table 1:

Example 79: 2-(3-(2-(dimethylamino)ethylamino)phenylamino)-N-(1,7,7-trimethylbicyclo[2.2.1]heptan-2-yl)pyrimidine-4-carboxyamide;
Example 80: 2-(3-(2-morpholinoethylamino)phenylamino)-N-(1,7,7-trimethylbicyclo[2.2.1]heptan-2-yl)pyrimidine-4-carboxyamide;
Example 81: 2-(3-(2-(pyridino-1-yl)ethylamino)phenylamino)-N-(1,7,7-trimethylbicyclo[2.2.1]heptan-2-yl)pyrimidine-4-carboxyamide;
Example 82: 2-(3-(2-(1-methylpyrimidino-2-yl)ethylamino)phenylamino)-N-(1,7,7-trimethylbicyclo[2.2.1]heptan-2-yl)pyrimidine-4-carboxyamide;
Example 86: 3-(4-dimethylaminoethylamino)-N-(1,7,7-trimethylbicyclo[2.2.1]heptan-2-yl)pyrimidine-4-carboxyamide;
Example 87: 3-(4-morpholinophenylamino)-N-(1,7,7-trimethylbicyclo[2.2.1]heptan-2-yl)pyrimidine-4-carboxyamide;
Example 88: 2-(4-(2-(pyrrolidin-1-yl)ethylamino)phenylamino)-N-(1,7,7-trimethylbicyclo[2.2.1]heptan-2-yl)pyrimidine-4-carboxyamide;
Example 90: 2-(4-(2-(piperidin-1-yl)ethoxy)phenylamino)-N-(1,7,7-trimethylbicyclo[2.2.1]heptan-2-yl)pyrimidine-4-carboxyamide;
Example 91: 2-(4-(2-(diethylamino)ethylamino)phenylamino)-N-(1,7,7-trimethylbicyclo[2.2.1]heptan-2-yl)pyrimidine-4-carboxyamide;
Example 94: 2-(3-(2-(diethylamino)ethylamino)phenylamino)-N-(1,7,7-trimethylbicyclo[2.2.1]heptan-2-yl)pyrimidine-4-carboxyamide;
Example 95: 2-(3-(2-(diethylamino)ethylamino)phenylamino)-N-(4-methylpentan-2-yl)pyrimidine-4-carboxyamide;
Example 96: 2-(3-(3-(phenylamino)phenylamino)-N-(1,7,7-trimethylbicyclo[2.2.1]heptan-2-yl)pyrimidine-4-carboxyamide;
Example 98: 2-(3-(3-morpholinopropylamino)phenylamino)-N-(1,7,7-trimethylbicyclo[2.2.1]heptan-2-yl)pyrimidine-4-carboxyamide;
Example 99: 2-(4-methoxy-3-(2-morpholinoethoxy)phenylamino)-N-(1,7,7-trimethylbicyclo[2.2.1]heptan-2-yl)pyrimidine-4-carboxyamide;
Example 102: 2-(4-(3-(dimethylamino)phenylamino)-N-(1,7,7-trimethylbicyclo[2.2.1]heptan-2-yl)pyrimidine-4-carboxyamide;
Example 106: 2-(3-(2-(diethylamino)ethoxy)-4-methoxyphenylamino)-N-(1,7,7-trimethylbicyclo[2.2.1]heptan-2-yl)pyrimidine-4-carboxyamide;
Example 107: 2-(4-methoxy-3-(2-(pyrrolidin-1-yl)ethoxy)phenylamino)-N-(1,7,7-trimethylbicyclo[2.2.1]heptan-2-yl)pyrimidine-4-carboxyamide;
Example 117: 2-(4-methoxy-3-(2-morpholinoethylamino)phenylamino)-N-(1,7,7-trimethylbicyclo[2.2.1]heptan-2-yl)pyrimidine-4-carboxyamide; and
Example 118: 2-(3-(cyanomethylamino)phenylamino)-N-(1,7,7-trimethylbicyclo[2.2.1]heptan-2-yl)pyrimidine-4-carboxyamide.

### Example 135

### 2-(3-(4-propylpiperazin-1-yl)phenylamino)-N-(1,7,7-trimethylbicyclo[2.2.1]heptan-2-yl)pyrimidine-4-carboxyamide

### Step 1) 1-(4-methoxybenzyl)-4-(3-nitrophenyl)piperazine

1-(3-nitrobenzyl)piperazine (Aldrich) (13.5 g) was dissolved in dimethyl formaldehyde (50 ml), sodium hydride (5.21 g) was slowly added thereto, followed by stirring the mixture at 0° C. for 10 minutes. p-methoxy benzylchloride (13.2 ml) was added thereto, and the reaction mixture was reacted at room temperature for 1 to 3 hours, concentrated under a reduced pressure to remove the solvent, and extracted with methylene chloride. The resulting residue was subjected to silica gel column chromatography (eluent: n-hexan/ethyl acetate=2:1) to obtain the title compound (20.7 g; yield: 71%).

### Step 2) 3-(4-(4-methoxybenzyl)piperazin-1-yl)aniline

The compound obtained in step 1 (20.5 g) was dissolved in ethanol (200 ml), to which raney nickel (20 ml) and hydrazine monohydrated (10 ml) was slowly added at 0° C. The reaction mixture was filtered with cellite to remove raney nickel, and concentrated under a reduced pressure to remove the solvent. The resulting residue was subjected to silica gel column chromatography (eluent: n-hexan/ethyl acetate=1:1) to obtain the title compound (17.42 g; yield: 94%).

### Step 3) 3-(4-(4-methoxybenzyl)piperazin-1-yl)phenylamino)pyrimidine-4-carbonitrile

The compound obtained in step 2 (16.5 g) and 2-chloropyrimidine-4-carbonitrile (7 g) were dissolved in 2-ethoxy ethanol (120 ml) and stirred, and the mixture was refluxed for 3 hours. The reaction mixture was concentrated under a reduced pressure to remove the solvent, and the resulting residue was subjected to silica gel column chromatography (eluent: methylene chloride/methanol=20:1) to obtain the title compound (18.8 g; yield: 84.6%).

### Step 4) 3-(4-(4-methoxybenzyl)piperazin-1-yl)phenylamino)pyrimidine-4-carboxylic acid

3-(4-(4-methoxybenzyl)piperazin-1-yl)phenylamino)pyrimidine-4-carbonitrile (18.7 g) was dissolved in methanol (40 ml), 3N NaOH was added thereto, and the mixture was refluxed with stirring. The reaction mixture was concentrated under a reduced pressure to remove the solvent, a small amount of water was added thereto, and pH of the mixture was adjusted with 1 N HCl to about 3 to 4 at 0° C. The resulting mixture was filtered to obtain a title compound as a yellow solid (15 g; yield: 76%).

### Step 5) 3-(4-(4-methoxybenzyl)piperazin-1-yl)phenylamino)-N-(1,7,7-trimethylbicyclo[2.2.1]heptan-2-yl)pyrimidine-4-carboxyamide

The compound obtained in step 4 (4.19 g), TBTU (3.84 g), triethylamine (2.7 ml) and carbonylamine (1.53 g) were dissolved in dimethyl formaldehyde and the mixture was stirred at room temperature for 2 hours. The reaction mixture was concentrated under a reduced pressure to remove the solvent, and extracted with methylene chloride (200 ml). The resulting residue was subjected to silica gel column chromatography (eluent: methylene chloride/methanol=20:1) to obtain the title compound (4.43 g; yield: 79%).

### Step 6) 2-(3-piperazin-1-yl)phenylamino)-N-(1,7,7-trimethylbicyclo[2.2.1]heptan-2-yl)pyrimidine-4-carboxyamide

The compound obtained in step 5 (4.43 g) was dissolved in methylene chloride (10 ml), trifluoro acetic acid (50 ml) was added thereto, and the mixture was refluxed for 6 hours. After neutralizing the reaction mixture by adding sodium bicarbonate aqueous solution, the reaction mixture was extracted with methylene chloride, and concentrated under a reduced pressure to remove the solvent. The resulting residue was subjected to silica gel column chromatography (eluent: methylene chloride containing a small amount of Et₃N/ethylacetate/methanol=30:10:1) to obtain the title compound (3.60 g; yield: 83%).

### Step 7) 2-(3-(4-propylpiperazin-1-yl)phenylamino)-N-(1,7,7-trimethylbicyclo[2.2.1]heptan-2-yl)pyrimidine-4-carboxyamide

The compound obtained in step 6 (473 mg), sodium bicarbonate (183.6 mg, 1.2 equivalents) and iodopropane (202 mg, 1.2 equivalents were dissolved in dimethyl formaldehyde (2 ml) and the resulting mixture was stirred at 30° C. for 3 hours. The reaction mixture was concentrated under a reduced pressure to remove the solvent, and extracted with methylene chloride. The resulting residue was subjected to silica gel column chromatography (eluent: methylene chloride containing a small amount of Et₃N/methanol=10:1) to obtain the title compound (418 mg; yield: 83%).

The procedure of Example 135 was repeated except for using each compounds corresponded to the compounds of formula 16a or 16b and R₂—R₃NH compound (R₂ and R₃ have the same meanings as defined above) as starting materials to prepare following compounds of Examples 89, 103 to 105, 108 to 110, 115, 116, and 120 to 137 listed in Table 1:

Example 89. 2-(4-(4-methylpiperazin-1-yl)phenylamino)-N-(1,7,7-trimethylbicyclo[2.2.1]heptan-2-yl)pyrimidine-4-carboxyamide;
Example 103. 2-(4-(piperazin-1-yl)phenylamino)-N-(1,7,7-trimethyl bicyclo[2.2.1]heptan-2-yl)pyrimidine-4-carboxyamide;
Example 104. 2-(4-(4-(4-methoxybenzyl)piperazin-1-yl)phenylamino)-N-(1,7,7-trimethylbicyclo[2.2.1]heptan-2-yl)pyrimidine-4-carboxyamide;
Example 105. 2-(4-(4-ethylpiperazin-1-yl)phenylamino)-N-(1,7,7-tri methylbicyclo[2.2.1]heptan-2-yl)pyrimidine-4-carboxyamide;
Example 108. 2-(4-(4-(2-hydroxyethyl)piperazin-1-yl)phenyl amino)-N-(1,7,7-trimethylbicyclo[2.2.1]heptan-2-yl)pyrimidine-4-carboxyamide;
Example 109. 2-(4-(4-isopropylpiperazin-1-yl)phenylamino)-N-(1,7,7-trimethylbicyclo[2.2.1]heptan-2-yl)pyrimidine-4-carboxyamide;
Example 110. 2-(4-(4-propylpiperazin-1-yl)phenylamino)-N-(1,7,7-trimethylbicyclo[2.2.1]heptan-2-yl)pyrimidine-4-carboxyamide;
Example 115. 2-(4-(4-isobutylpiperazin-1-yl)phenylamino)-N-(1,7,7-trimethylbicyclo[2.2.1]heptan-2-yl)pyrimidine-4-carboxyamide;
Example 116. 2-(4-(4-(cianomethyl)piperazin-1-yl)phenylamino)-N-(1,7,7-trimethylbicyclo[2.2.1]heptan-2-yl)pyrimidine-4-carboxyamide;
Example 120. N-(bicyclo[2.2.1]heptan-2-yl)-2-(4-(piperazin-1-yl)phenylamino)-N-(1,7,7-trimethylbicyclo[2.2.1]heptan-2-yl)pyrimidine-4-carboxyamide;
Example 121. N-(bicyclo[2.2.1]heptan-2-yl)-2-(4-(4-propylpiperazin-1-yl)phenylamino)-N-(1,7,7-trimethylbicyclo[2.2.1]heptan-2-yl)pyrimidine-4-carboxyamide(endo);
Example 122. N-(bicyclo[2.2.1]heptan-2-yl)-2-(4-(4-hydroxy propyl piperazin-1-yl)phenylamino)-N-(1,7,7-trimethylbicyclo[2.2.1]heptan-2-yl)pyrimidine-4-carboxyamide(endo);
Example 123. N-(bicyclo[2.2.1]heptan-2-yl)-2-(4-(piperazin-1-yl)phenylamino)pyrimidine-4-carboxyamide;
Example 124. N-(bicyclo[2.2.1]heptan-2-yl)-2-(4-(4-4-propyl piperazin-1-yl)phenylamino)pyrimidine-4-carboxyamide(exo);
Example 125. N-(bicyclo[2.2.1]heptan-2-yl)-2-(4-(4-(2-hydroxyethyl)piperazin-1-yl)phenylamino)pyrimidine-4-carboxyamide;
Example 126. N-(pentane-3-yl)-2-(4-(piperazin-1-yl)phenylamino)pyrimidine-4-carboxyamide;
Example 127. N-(pentane-3-yl)-2-(4-(4-propylpiperazin-1-yl)phenyl amino)pyrimidine-4-carboxyamide;
Example 128. 2-(4-(4-(2-hydroxyethyl)piperazin-1-yl)phenylamino)-N-(pentane-3-yl)pyrimidine-4-carboxyamide;
Example 129. N-tert-phenyl-2-(4-(piperazin-1-yl)phenylamino)pyrimidine-4-carboxyamide;
Example 130. 2-(4-(4-propylpiperazin-1-yl)phenylamino)-N-tert-pentylpyrimidine-4-carboxyamide;
Example 131. 2-(4-(4-(2-hydroxyethyl)piperazin-1-yl)phenylamino)-N-tert-pentylpyrimidine-4-carboxyamide;
Example 132. N,N-diethyl-2-(4-(piperazin-1-yl)phenylamino)pyrimidine-4-carboxyamide;
Example 133. N,N-diethyl-2-(4-(4-propylpiperazin-1-yl)phenylamino)pyrimidine-4-carboxyamide;
Example 134. N,N-diethyl-2-(4-(4-(2-hydroxyethyl)piperazin-1-yl)phenylamino)pyrimidine-4-carboxyamide;
Example 136. 2-(3-(4-isobutylpiperazin-1-yl)phenylamino)-N-(1,7,7-trimethylbicyclo[2.2.1]heptan-2-yl)pyrimidine-4-carboxyamide; and
Example 137. 2-(3-(4-isopropylpiperazin-1-yl)phenylamino)-N-(1,7,7-trimethylbicyclo[2.2.1]heptan-2-yl)pyrimidine-4-carboxyamide.

### Test Example

### Inhibitory Effect of the Inventive Aminopyrimidine Derivatives on the Protein Kinase Activity

In order to examine the suppressive effect of the inventive aminopyrimidine derivatives on the proliferation of cancer cells through inhibiting the protein kinase activity, a test was performed as follows by employing human colon carcinoma cell line, HCT116 (Korean Cell Line Bank, KCLB10247) and human fetal cell line, MRC-5 (Korean Cell Line Bank, KCLB10171).

HCT116 and MRC-5 cells were plated into the wells of a 96-well plate containing DMEM (Dulbecco's Modified Eagle Medium) medium at a concentration ranging from 1×10³ to 3×10³ cells/well, and cultured under conditions of 5% CO₂ and 37° C. for 24 hours. Then, the test compounds of Examples 85 to 88, 97 to 99, 105 to 110 and 115 to 118, and a comparative compound, N-(4-(4-(5-methyl-1H-pyrrole-3-yl-amino)-6-(4-methylpiperazin-1-yl)-1,3,5-triazin-2-ylthio) cyclopropane carboxyamide were added thereto in an amount of 0.2, 1, 5, 25 and 100 μM, respectively. The control was not treated with any compound. The cells were cultured for 48 hours.

After removing the medium from each wells, the cells were washed about 3 times with phosphate-buffered saline (PBS, pH 7.4), followed by fixing with 50% TCA (trichloroacetic acid) refrigerant solution in an amount of 50 μl/well at 4° C. for 1 hour. The fixed cells were washed 5 times with distilled water and dried in the air, and stained with 50 μl/well of 1% acetic acid containing 0.4% SRB (sulforhodamine B) solution at room temperature for 1 hour. The stained cells were washed 5 times with 1% acetic acid and dried in the air, 150 μl/well of 10 mM Tris-HCl (pH 10.5) were added thereto, and the absorbance of each well was measured at 540 nm. The suppressive value of each compound for cell proliferation was calculated based on the absorbance of the control well, and the results are shown in Table 2. In Table 2, EC₅₀ (μM) was determined as a concentration of the test compound required to inhibit cancer cell proliferation by 50% relative to the control, CC₅₀ (μM) was determined as a concentration of the test compound required to inhibit normal cell proliferation by 50% relative to the control, and the therapeutic index was determined as the value of CC₅₀(μM)/EC₅₀(μM).

Further, the test compounds and comparative compound were successively diluted with dimethyl sulfoxide (DMSO) from an initial concentration of 12.5 mM to obtain test solutions and DMSO was used as a control solution. Less than 5% of each test solution or control solution was added to a reaction solution containing 20 mM HEPES (pH 7.5), 5 mM MgCl₂, 0.5 mM EGTA, 200 mM KCl, 1 mM DTT and 0.05% triton X-100, and 100 μM of Kemptide peptide (PEPTRON), which is a substrate of aurora kinase, 1 μM of ATP and 10 nM of Aurora A (Upstate), which is a recombinant aurora kinase, were added thereto, followed by reacting each mixture at 30° C. for 1 hour. 25 μl of each reacted solution was mixed with 25 μl of Kinase-Glo™ (promega), and reacted at room temperature for 10 minutes, and the amount of residual ATP therein was measured using Fusion a-FP (Packard). Enzymic activity in each reacted solution was analyzed based on the measured value, and IC₅₀ (μM) was determined as a concentration of the test compound required to inhibit the kinase activity by 50% relatively to the control solution, and the results are shown in Table 2.

TABLE 2

| | | | | Therapeutic |
|---|---|---|---|---|
| Compounds | IC₅₀(μM) | EC₅₀(μM) | CC₅₀(μM) | index |
| Compound 106 | 0.011 | 0.9 | 33.0 | 67.6 |
| Compound 107 | 0.018 | 1.2 | 46.0 | 38.0 |
| Compound 105 | 0.017 | 0.5 | >100 | >100 |
| Compound 110 | 0.020 | 1.9 | 32.0 | 16.8 |
| Compound 109 | 0.020 | 2.8 | 23.5 | 8.4 |
| Compound 85 | 0.033 | 1.6 | 38.0 | 23.8 |
| Compound 115 | 0.093 | 3.1 | 100 | 32.4 |
| Compound 108 | 0.010 | 1.9 | 56.0 | 29.4 |
| Compound 116 | 0.012 | 1.7 | 26.6 | 15.6 |
| Compound 118 | 0.019 | 2.8 | 62.5 | 22.4 |
| Compound 87 | 0.053 | 2.0 | 18.0 | 9.0 |
| Compound 97 | 0.050 | 3.1 | >100 | >33.3 |
| Compound 88 | 0.010 | 0.8 | 24.5 | 30.6 |
| Compound 86 | 0.088 | 1.2 | >100 | >83 |
| Compound 98 | 0.039 | 3.2 | 52.6 | 16.4 |
| Compound 99 | 0.023 | 2.0 | 52.0 | 26.0 |
| Comparative | 0.019 | 3.0 | 33 | 11 |
| compound | | | | |

As shown in table 2, the compounds of the present invention effectively inhibit the activity of aurora kinase, and suppress proliferation of a cancer cell predominantly as compared with normal cells. Namely, the aminopyrimidine derivative of the present invention can effectively inhibit abnormal activity of protein kinases in cancer cells to prevent or treat the cancer.

While the invention has been described with respect to the above specific embodiments, it should be recognized that various modifications and changes may be made to the invention by those skilled in the art which also fall within the scope of the invention as defined by the appended claims.

1. A compound of formula 1, or a pharmaceutically acceptable salt, hydrate, solvate or isomer thereof: 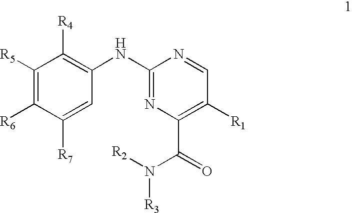
wherein,
R₁ is hydrogen, hydroxy, halogen, C₁₋₂ alkyloxy or C₁₋₂ alkyl;
R₂ is unsubstituted or substituted C₁₋₈ alkyl or C₂₋₈ alkenyl, unsubstituted or substituted C₁₋₈ alkyl or C₂₋₈ alkenyl comprising one or more nitrogen, sulfur or oxygen in its chain structure, the substituent of the alkyl or alkenyl being hydroxy; halogen; C₁₋₆ alkyloxy; C₁₋₆ alkyl; aminoalkyl; C₁₋₆ alkylamine; acetylamino; carboxyl; nitro; sulfonylamino; C₁₋₆ alkylsulfonyl; aryl optionally substituted with hydroxy, halogen, C₁₋₆ alkyloxy, C₁₋₆ alkyl, amino, C₁₋₆ alkylamino, acetylamino, carboxyl, nitro, amide, dimethyl sulfoneamino or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro or amide; aryl comprising nitrogen, sulfur or oxygen in its ring structure represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, which is unsubstituted, or substituted with hydroxy, halogen, C₁₋₆ alkyloxy, C₁₋₆ alkyl, amino, C₁₋₆ alkylamino, carbonylamino, carboxyl, nitro, C₁₋₆ trihaloalkane, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carbonyl amino, carboxyl, nitro or amide; or
unsubstituted or substituted aryl, or unsubstituted or substituted aryl comprising one or more nitrogen, sulfur or oxygen in its ring structure represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, the substituent thereof being hydroxy; halogen; C₁₋₆ alkyloxy; C₁₋₆ alkyl; amino; C₁₋₆ alkylamino; carboxyl; nitro; C₁₋₆ trihaloalkane; sulfonylamide; C₁₋₆ alkylsulfonyl; C₁₋₆ alkyl, or C₃₋₈ cycloalkyl having optional one or more nitrogen, sulfur or oxygen atoms in its chain structure as well as optional substituent selected from the group consisting of hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl and amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, represented by pyrrole, pyrazole, imidazole, 1,2,3-triazole, 1,2,4-triazole, furan, isooxazole, oxazole, thiophene, isothiazole, thiazolidine, thiazole, 1,2,5-oxadiazole, 1,2,3-oxadiazole, 1,2,5-thiodiazole, 1,2,3-thiodiazole, 1,3,4-oxadiazole, 1,3,4-thiodiazole, pyridine, pyrimidine, tetrazole or triazine, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide;
R₃ is hydrogen; hydroxy; unsubstituted or substituted C₁₋₈ alkyl or C₃₋₈ cycloalkyl optionally having one or more nitrogen, sulfur or oxygen atoms in its chain structure, the substituent of the alkyl or cycloalkyl being hydroxyl; halogen; C₁₋₆ alkyloxy; C₁₋₆ alkyl; amino; C₁₋₆ alkylamino; carboxyl; nitro; sulfonylamide; C₁₋₆ alkylsulfonyl; amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide; or
R₂ and R₃ are fused together with the nitrogen to which they are attached to form a morpholine ring, and
R₄, R₅, R₆ and R₇ are each independently hydrogen, hydroxy, halogen, amine substituted with C₁₋₆ alkyl or C₃₋₆ cycloalkyl having optional substituent, or amine substituted with C₁₋₈ alkyl or C₃₋₆ cycloalkyl comprising one or more nitrogen, sulfur or oxygen in its chain structure, the substituent of the alkyl or cycloalkyl being hydroxyl; halogen; C₁₋₆ alkyloxy; C₁₋₆ alkyl; amino; C₁₋₆ alkylamino; carboxyl; nitro; sulfonylamide; C₁₋₆ alkylsulfonyl; amide; aryl optionally substituted with hydroxy, halogen, C₁₋₆ alkyloxy, C₁₋₆ alkyl, amino, C₁₋₆ alkylamino, carboxyl, nitro, amide or dioxoisoindole; sulfonylaminoaryl having an aryl group substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; aryl comprising nitrogen, sulfur or oxygen in its ring structure, which is optionally substituted with hydroxy, halogen, C₁₋₆ alkyloxy, C₁₋₆ alkyl, amino, C₁₋₆ alkylamino, carboxyl, nitro, sulfonylamide, C₁₋₆ alkylsulfonyl or amide; or C₃₋₈ cycloalkyl optionally substituted with hydroxy, halogen, C₁₋₆ alkyloxy, C₁₋₆ alkyl, amino, C₁₋₆ alkylamino, carboxyl, nitro or amide; or
R₆ is fused together with R₅ or R₇ to form a dioxorane ring.

2. The compound of claim 1, wherein R₁ is H, Cl or Br; R₂ is H, or unsubstituted or substituted C₁₋₆ alkyl; R₃ is H, or unsubstituted or substituted C₁₋₆ alkyl; R₄ is aminoC₁₋₆ alkylamine substituted with halogen (e.g., F or Cl), C₁₋₆ alkoxy or substituted C₁₋₆ alkyl; R₅ is amino C₁₋₆ alkylamine substituted with halogen (e.g., F or Cl), C₁₋₆ alkoxy, substituted C₁₋₆ alkyl or substituted ring compound; R₆ is aminoC₁₋₆ alkylamine substituted with halogen (e.g., F or Cl), C₁₋₆ alkoxy or substituted C₁₋₆ alkyl; R₇ is amino C₁₋₆ alkylamine substituted with halogen (e.g., F or Cl), C₁₋₆ alkoxy or substituted C₁₋₆ alkyl.

3. The compound of claim 1, which is selected from the group consisting of:
1) 2-(3-ethoxy-phenylamino)-pyrimidine-4-carboxylic acid cyclohexylamide;
2) 2-phenylamino-pyrimidine-4-carboxylic acid[3-(4,5-dichloro-imidazol-1-yl)-propyl]-amide;
3) 2-(3-fluoro-phenylamino)-pyrimidine-4-carboxylic acid[2-(4-ethylsulfonylamino-phenyl)-ethyl]-amide;
4) 2-(4-fluoro-phenylamino)-pyrimidine-4-carboxylic acid[2-(4-ethylsulfonylamino-phenyl)-ethyl]-amide;
5) 2-(2,4-difluoro-phenylamino)-pyrimidine-4-carboxylic acid[2-(4-ethylsulfonylamino-phenyl)-ethyl]-amide;
6) 2-(4-chloro-phenylamino)-pyrimidine-4-carboxylic acid[2-(4-ethylsulfonylamino-phenyl)-ethyl]-amide;
7) 2-(3,4-difluoro-phenylamino)-pyrimidine-4-carboxylic acid[2-(4-ethylsulfonylamino-phenyl)-ethyl]-amide;
8) 2-(3,5-difluoro-phenylamino)-pyrimidine-4-carboxylic acid[2-(4-ethylsulfonylamino-phenyl)-ethyl]-amide
9) 2-[4-(2-amino-ethyl)-phenylamino]-pyrimidine-4-carboxylic acid[2-(4-ethylsulfonylamino-phenyl)-ethyl]-amide;
10) 2-(3-methoxy-phenylamino)-pyrimidine-4-carboxylic acid(2-pyridine-3-yl-ethyl)-amide;
11) 2-(4-methoxy-phenylamino)-pyrimidine-4-carboxylic acid(2-pyridine-4-yl-ethyl)-amide;
12) 2-(4-methoxy-phenylamino)-pyrimidine-4-carboxylic acid(2-pyridine-3-yl-ethyl)-amide;
13) 2-(4-methoxy-phenylamino)-pyrimidine-4-carboxylic acid[2-(2-hydroxy-phenyl)-ethyl]-amide;
14) 2-(4-methoxy-phenylamino)-pyrimidine-4-carboxylic acid[2-(3-hydroxy-phenyl)-ethyl]-amide;
15) 2-(4-methoxy-phenylamino)-pyrimidine-4-carboxylic acid(2-{4-[2-(4-ethyl-piperazin-1-yl)-acetylamino]-phenyl}-ethyl)-amide;
16) 2-(4-methoxy-phenylamino)-pyrimidine-4-carboxylic acid[2-(4-acetylamino-phenyl)-ethyl]-amide;
17) 2-(4-methoxy-phenylamino)-pyrimidine-4-carboxylic acid[2-(4-morpholine-4-yl-phenyl)-ethyl]-amide;
18) 2-(4-methoxy-phenylamino)-pyrimidine-4-carboxylic acid{2-[4-(2-dimethylamino-acetylamino)-phenyl]-ethyl}-amide;
19) 2-(4-methoxy-phenylamino)-pyrimidine-4-carboxylic acid[3-(2-methyl-imidazol-1-yl)-propyl]-amide;
20) 2-(3,5-difluoro-phenylamino)-N-(2-(pyridine-4-yl)ethyl)pyrimidine-4-carboxyamide;
21) 2-(4-hydroxy-phenylamino)-N-(2-(pyridin-2-yl)ethyl)pyrimidine-4-carboxyamide;
22) 2-(4-hydroxy-phenylamino)-pyrimidine-4-carboxylic acid(2-pyridine-3-yl-ethyl)-amide;
23) 2-(3,5-difluoro-phenylamino)-N-(3-fluoro-4-hydroxy)pyrimidine-4-carboxyamide;
24) methyl-5-chloro-2-(3-fluorophenylamino)pyrimidine-4-carboxylate;
25) 2-(benzo[1,3]dioxol-5-ylamino)-5-chloro-pyrimidine-4-carboxylic acid(3-phenyl-propyl)-amide;
26) 5-chloro-2-phenylamino-pyrimidine-4-carboxylic acid(3-phenyl-propyl)-amide;
27) 2-(benzole[1,3]dioxol-5-ylamino)-5-chloro-pyrimidine-4-carboxylic acid[3-(4,5-dichloro-imidazol-1-yl)-propyl]-amide;
28) 5-chloro-2-(3-methoxy-phenylamino)-pyrimidine-4-carboxylic acid[3-(4,5-dichloro-imidazol-1-yl)-propyl]-amide;
29) 5-chloro-2-phenylamino-pyrimidine-4-carboxylic acid [3-(4,5-dichloro-imidazol-1-yl)-propyl]-amide;
30) 2-(benzo[d][1,3]dioxalyl-5-amino)-5-chloro-N-methylpyrimidine-4-carboxyamide;
31) 5-chloroN-(3-(4,5-dichloro-1H-imidazolyl-1-yl)propyl)-2-(3-methoxyphenylamino)pyrimidine-4-carboxyamide;
32) 5-chloro-2-phenylamino-pyrimidine-4-carboxylic acid cyclohexylamide;
33) 2-(benzo[1,3]dioxol-5-ylamino)-5-chloro-pyrimidine-4-carboxylic acid cyclohexylamide;
34) 5-chloro-2-(3-ethoxy-phenylamino)-pyrimidine-4-carboxylic acidcycloamide;
35) 2-phenylamino-pyrimidine-4-carboxylic acid[2-(4-ethylsulfonylamino-phenyl)-ethyl]-amide;
36) 5-chloro-2-(3-methoxy-phenylamino)-pyrimidine-4-carboxylic acid(4-amino-cyclohexyl)-amide;
37) 2-(3-ethoxy-phenylamino)-pyrimidine-4-carboxylic acid[3-(4,5-dichloro-imidazol-1-yl)-propyl]-amide;
38) 2-(3-benzylalkyl-phenylamino)-pyrimidine-4-carboxylic acid cyclehexyloamide;
39) 2-(3-benzylalkyl-phenylamino)-pyrimidine-4-carboxylic acid[3-(4,5-dichloro-imidazol-1-yl)-propyl]-amide;
40) 2-(3-ethoxy-phenylamino)-pyrimidine-4-carboxylic acid(4-aminocyclohexyl)-amide;
41) 2-(3-ethoxy-phenylamino)-pyrimidine-4-carboxylic acid[4-(cyclopropanecarbonyl-amino)-cyclohexyl]-amide;
42) 5-chloro-2-(3-methoxy-phenylamino)-pyrimidine-4-carboxylic acid[4-(2,2-dimethyl-propionylamino)-cyclohexyl]-amide;
43) 2-[4-methoxy-3-(2-morpholine-4-yl-ethylamino)-phenylamino]-pyrimidine-4-carboxylic acid cyclohexylamide;
44) 2-(3-acetylamino-phenylamino)-pyrimidine-4-carboxylic acid cycloamide;
45) 2-(3,5-dimethoxy-phenylamino)-pyrimidine-4-carboxylic acid cycloamide;
46) 5-chloro-2-(3-methoxy-phenylamino)-pyrimidine-4-carboxylic acid(3-pyrrolidin-1-yl-propyl)-amide;
47) 5-chloro-2-(3-methoxy-phenylamino)-pyrimidine-4-carboxylic acid[3-(4-methyl-piperazin-1-yl)-propyl]-amide;
48) 5-chloro-2-(3-methoxy-phenylamino)-pyrimidine-4-carboxylic acid(1-methyl-1-phenyl-ethyl)-amide;
49) 5-chloro-2-(3-methoxy-phenylamino)-pyrimidine-4-carboxylic acid[4-(propane-2-sulfonylamino)-cyclohexyl]-amide;
50) 2-(3-ethoxy-phenylamino)-pyrimidine-4-carboxylic acid(3-aminocyclohexyl)-amide;
51) 5-chloro-2-(3-methoxy-phenylamino)-pyrimidine-4-carboxylic acid(4-tert-butyl-cyclohexyl)-amide;
52) 5-chloro-2-(3-methoxy-phenylamino)-pyrimidine-4-carboxylic acid cycloheptylamide;
53) 5-chloro-2-(3-methoxy-phenylamino)-pyrimidine-4-carboxylic acid(2,2,6,6-tetramethyl-piperidine-4-yl)-amide;
54) 5-chloro-2-(3-methoxy-phenylamino)-pyrimidine-4-carboxylic acid inden-2-yl amide;
55) 5-chloro-2-(3-methoxy-phenylamino)-pyrimidine-4-carboxylic acid(1,2,3,4-tetrahydro-naphthalen-1-yl)-amide;
56) 5-chloro-2-(3-methoxy-phenylamino)-pyrimidine-4-carboxylic acid[1-(4-chloro-phenyl)-propyl]-amide;
57) 5-chloro-2-(3-methoxy-phenylamino)-pyrimidine-4-carboxylic acid sec-butylamide;
58) 2-(3-ethoxy-phenylamino)-pyrimidine-4-carboxylic acid(2,6-dimethyl-phenyl)-amide;
59) 2-(3-ethoxy-phenylamino)-pyrimidine-4-carboxylic acid[3-(cyclopropane carbonyl-amino)-cyclohexyl]-amide;
60) 2-(3-ethoxy-phenylamino)-pyrimidine-4-carboxylic acid(3-acetylamino-cyclohexyl)-amide;
61) 2-(3-ethoxy-phenylamino)-pyrimidine-4-carboxylic acid {3-[(thiophene-3-carbonyl)-amino]-cyclohexyl}-amide;
62) 2-(3-methoxy-phenylamino)-pyrimidine-4-carboxylic acid benzyl-ethyl-amide;
63) 2-(3-methoxy-phenylamino)-pyrimidine-4-carboxylic acid(2-amino-cyclohexyl)-amide;
64) 2-(3-ethoxy-phenylamino)-pyrimidine-4-carboxylic acid[2-(cyclopropanecarbonyl-amino)-cyclohexyl]-amide;
65) 2-(3-methoxy-phenylamino)-pyrimidine-4-carboxylic acid(2-butylamino-cyclohexyl)-amide;
66) 2-(3-methoxy-phenylamino)-pyrimidine-4-carboxylic acid[2-(2-chloro-acetylamino)-cyclohexyl]-amide;
67) 2-(3-methoxy-phenylamino)-pyrimidine-4-carboxylic acid(2-propylamino-cyclohexyl)-amide;
68) 2-(3-methoxy-phenylamino)-pyrimidine-4-carboxylic acid(4,7,7-trimethyl-bicyclo [2.2.1]heptan-2-yl)-amide;
69) 2-(3-methoxy-phenylamino)-pyrimidine-4-carboxylic acidbiphenyl-2-yl amide;
70) 2-(3-methoxy-phenylamino)-pyrimidine-4-carboxylic acid(2-dipropylamino-cyclohexyl)-amide;
71) 2-(3-methoxy-phenylamino)-pyrimidine-4-carboxylic acid(2-ethyl-6-methyl-phenyl)-amide;
72) 2-(3-methoxy-phenylamino)-pyrimidine-4-carboxylic acid aryl-phenyl-amide;
73) 2-(3-methoxy-phenylamino)-pyrimidine-4-carboxylic acid cyclohexyl-ethyl-amide;
74) 2-(3-methoxy-phenylamino)-pyrimidine-4-carboxylic acid(2-amino-cyclohexyl)-amide;
75) 2-(3-methoxy-phenylamino)-pyrimidine-4-carboxylic acid[2-(cyclopropanecarbonyl-amino)-cyclohexyl]-amide;
76) 2-(3-fluoro-phenylamino)-pyrimidine-4-carboxylic acid cyclohexyl amide;
77) 2-(3-amino-phenylamino)-pyrimidine-4-carboxylic acid cyclohexylamide;
78) 2-(3-amino-phenylamino)-pyrimidine-4-carboxylic acid(1,7,7-trimethyl-bicyclo [2.2.1]heptan-2-yl)-amide;
79) 2-(3-(2-(dimethylamino)ethylamino)phenylamino)-N-(1,7,7-trimethylbicyclo[2.2.1]heptan-2-yl)pyrimidine-4-carboxyamide;
80) 2-(3-(2-morpholinoethylamino)phenylamino)-N-(1,7,7-trimethylbicyclo[2.2.1]heptan-2-yl)pyrimidine-4-carboxyamide;
81) 2-(3-(2-(pyridino-1-yl)ethylamino)phenylamino)-N-(1,7,7-trimethylbicyclo[2.2.1]heptan-2-yl)pyrimidine-4-carboxyamide;
82) 2-(3-(2-(1-methylpyrimidino-2-yl)ethylamino)phenylamino)-N-(1,7,7-trimethylbicyclo[2.2.1]heptan-2-yl)pyrimidine-4-carboxyamide;
83) 2-(4-morpholinophenylamino)-N-(1,7,7-trimethylbicyclo[2.2.1]heptan-2-yl)pyrimidine-4-carboxyamide;
84) 2-(4-aminophenylamino)-N-(1,7,7-trimethylbicyclo[2.2.1]heptan-2-yl)pyrimidine-4-carboxyamide;
85) 2-(4-(piperidin-1-yl)phenylamino)-N-(1,7,7-trimethylbicyclo[2.2.1]heptan-2-yl)pyrimidine-4-carboxyamide;
86) 3-(4-dimethylaminoethylamino)-N-(1,7,7-trimethylbicyclo[2.2.1]heptan-2-yl)pyrimidine-4-carboxyamide;
87) 3-(4-morpholinophenylamino)-N-(1,7,7-trimethylbicyclo[2.2.1]heptan-2-yl)pyrimidine-4-carboxyamide;
88) 2-(4-(2-(pyrrolidin-1-yl)ethylamino)phenylamino)-N-(1,7,7-trimethylbicyclo[2.2.1]heptan-2-yl)pyrimidine-4-carboxyamide;
89) 2-(4-(4-methylpiperazin-1-yl)phenylamino)-N-(1,7,7-trimethylbicyclo[2.2.1]heptan-2-yl)pyrimidine-4-carboxyamide;
90) 2-(4-(2-(piperidin-1-yl)ethoxy)phenylamino)-N-(1,7,7-trimethylbicyclo[2.2.1]heptan-2-yl)pyrimidine-4-carboxyamide;
91) 2-(4-(2-(diethylamino)ethylamino)phenylamino)-N-(1,7,7-trimethylbicyclo[2.2.1]heptan-2-yl)pyrimidine-4-carboxyamide;
92) 2-(4-(dimethylamino)ethylamino)phenylamino)-N-(1,7,7-trimethylbicyclo[2.2.1]heptan-2-yl)pyrimidine-4-carboxyamide;
93) 2-(3-(dimethylamino)ethylamino)phenylamino)-N-(1,7,7-trimethylbicyclo[2.2.1]heptan-2-yl)pyrimidine-4-carboxyamide;
94) 2-(3-(2-(diethylamino)ethylamino)phenylamino)-N-(1,7,7-trimethylbicyclo[2.2.1]heptan-2-yl)pyrimidine-4-carboxyamide;
95) 2-(3-(2-(diethylamino)ethylamino)phenylamino)-N-(4-methylpentan-2-yl)pyrimidine-4-carboxyamide;
96) 2-(3-(3-(phenylamino)phenylamino)-N-(1,7,7-trimethylbicyclo[2.2.1]heptan-2-yl)pyrimidine-4-carboxyamide;
97) 2-(3-(3-morpholinopropylamino)phenyl amino)-N-1-(1,7,7-trimethoxybicyclo[2.2.1]heptan-2-yl)pyrimidine-4-carboxyamide;
98) 2-(3-(3-morpholinopropylamino)phenylamino)-N-(1,7,7-trimethylbicyclo[2.2.1]heptan-2-yl)pyrimidine-4-carboxyamide;
99) 2-(4-methoxy-3-(2-morpholinoethoxy)phenylamino)-N-(1,7,7-trimethylbicyclo[2.2.1]heptan-2-yl)pyrimidine-4-carboxyamide;
100) 2-(3-sulfamoylphenylamino)-N-(1,7,7-trimethylbicyclo[2.2.1]heptan-2-yl)pyrimidine-4-carboxyamide;
101) 2-(4-sulfamoylphenylamino)-N-(1,7,7-trimethylbicyclo[2.2.1]heptan-2-yl)pyrimidine-4-carboxyamide;
102) 2-(4-(3-(dimethylamino)phenylamino)-N-(1,7,7-trimethylbicyclo[2.2.1]heptan-2-yl)pyrimidine-4-carboxyamide;
103) 2-(4-(piperazin-1-yl)phenylamino)-N-(1,7,7-trimethylbicyclo[2.2.1]heptan-2-yl)pyrimidine-4-carboxyamide;
104) 2-(4-(4-(4-methoxybenzyl)piperazin-1-yl)phenylamino)-N-(1,7,7-trimethylbicyclo[2.2.1]heptan-2-yl)pyrimidine-4-carboxyamide;
105) 2-(4-(4-ethylpiperazin-1-yl)phenylamino)-N-(1,7,7-trimethylbicyclo[2.2.1]heptan-2-yl)pyrimidine-4-carboxyamide;
106) 2-(3-(2-(diethylamino)ethoxy)-4-methoxyphenylamino)-N-(1,7,7-trimethylbicyclo[2.2.1]heptan-2-yl)pyrimidine-4-carboxyamide;
107) 2-(4-methoxy-3-(2-(pyrrolidin-1-yl)ethoxy)phenylamino)-N-(1,7,7-trimethylbicyclo[2.2.1]heptan-2-yl)pyrimidine-4-carboxyamide;
108) 2-(4-(4-(2-hydroxyethyl)piperazin-1-yl)phenylamino)-N-(1,7,7-trimethylbicyclo[2.2.1]heptan-2-yl)pyrimidine-4-carboxyamide;
109) 2-(4-(4-isopropylpiperazin-1-yl)phenylamino)-N-(1,7,7-trimethylbicyclo[2.2.1]heptan-2-yl)pyrimidine-4-carboxyamide;
110) 2-(4-(4-propylpiperazin-1-yl)phenylamino)-N-(1,7,7-trimethylbicyclo[2.2.1]heptan-2-yl)pyrimidine-4-carboxyamide;
111) 2-(4-(diethylamino)phenylamino)-N-(1,7,7-trimethylbicyclo[2.2.1]heptan-2-yl)pyrimidine-4-carboxyamide;
112) 2-(2-(2-morpholinoethoxy)phenylamino)-N-(1,7,7-trimethylbicyclo[2.2.1]heptan-2-yl)pyrimidine-4-carboxyamide;
113) 2-(2-(2-(pyrrolidin-1-yl)ethoxy)phenylamino)-N-(1,7,7-trimethylbicyclo[2.2.1]heptan-2-yl)pyrimidine-4-carboxyamide;
114) 2-(3-(morpholine-4-carbonyl)phenylamino)pyrimidine-4-yl)(morpholino)methanone;
115) 2-(4-(4-isobutylpiperazin-1-yl)phenylamino)-N-(1,7,7-trimethylbicyclo[2.2.1]heptan-2-yl)pyrimidine-4-carboxyamide;
116) 2-(4-(4-(cianomethyl)piperazin-1-yl)phenylamino)-N-(1,7,7-trimethylbicyclo[2.2.1]heptan-2-yl)pyrimidine-4-carboxyamide;
117) 2-(4-methoxy-3-(2-morpholinoethylamino)phenylamino)-N-(1,7,7-trimethylbicyclo[2.2.1]heptan-2-yl)pyrimidine-4-carboxyamide;
118) 2-(3-(cianomethylamino)phenylamino)-N-(1,7,7-trimethylbicyclo[2.2.1]heptan-2-yl)pyrimidine-4-carboxyamide;
119) 2-(5-methoxy-2-(2-morpholinoethoxy)phenylamino)-N-(1,7,7-trimethylbicyclo[2.2.1]heptan-2-yl)pyrimidine-4-carboxyamide;
120) N-(bicyclo[2.2.1]heptan-2-yl)-2-(4-(piperazin-1-yl)phenylamino)-N-(1,7,7-trimethylbicyclo[2.2.1]heptan-2-yl)pyrimidine-4-carboxyamide;
121) N-(bicyclo[2.2.1]heptan-2-yl)-2-(4-(4-propylpiperazin-1-yl)phenylamino)-N-(1,7,7-trimethylbicyclo[2.2.1]heptan-2-yl)pyrimidine-4-carboxyamide(endo);
122) N-(bicyclo[2.2.1]heptan-2-yl)-2-(4-(4-hydroxy propylpiperazin-1-yl)phenylamino)-N-(1,7,7-trimethylbicyclo[2.2.1]heptan-2-yl)pyrimidine-4-carboxyamide(endo);
123) N-(bicyclo[2.2.1]heptan-2-yl)-2-(4-(piperazin-1-yl)phenylamino)pyrimidine-4-carboxyamide;
124) N-(bicyclo[2.2.1]heptan-2-yl)-2-(4-(4-4-propylpiperazin-1-yl)phenylamino)pyrimidine-4-carboxyamide(exo);
125) N-(bicyclo[2.2.1]heptan-2-yl)-2-(4-(4-(2-hydroxyethyl)piperazin-1-yl)phenylamino)pyrimidine-4-carboxyamide;
126) N-(pentane-3-yl)-2-(4-(piperazin-1-yl)phenylamino)pyrimidine-4-carboxyamide;
127) N-(pentane-3-yl)-2-(4-(4-propylpiperazin-1-yl)phenylamino)pyrimidine-4-carboxyamide;
128) 2-(4-(4-(2-hydroxyethyl)piperazin-1-yl)phenylamino)-N-(pentane-3-yl)pyrimidine-4-carboxyamide;
129) N-tert-phenyl-2-(4-(piperazin-1-yl)phenylamino)pyrimidine-4-carboxyamide;
130) 2-(4-(4-propylpiperazin-1-yl)phenylamino)-N-tert-pentylpyrimidine-4-carboxyamide;
131) 2-(4-(4-(2-hydroxyethyl)piperazin-1-yl)phenylamino)-N-tert-pentylpyrimidine-4-carboxyamide;
132) N,N-diethyl-2-(4-(piperazin-1-yl)phenylamino)pyrimidine-4-carboxyamide;
133) N,N-diethyl-2-(4-(4-propylpiperazin-1-yl)phenylamino)pyrimidine-4-carboxyamide;
134) N,N-diethyl-2-(4-(4-(2-hydroxyethyl)piperazin-1-yl)phenylamino)pyrimidine-4-carboxyamide;
135) 2-(3-(4-propylpiperazin-1-yl)phenylamino)-N-(1,7,7-trimethylbicyclo[2.2.1]heptan-2-yl)pyrimidine-4-carboxyamide;
136) 2-(3-(4-isobutylpiperazin-1-yl)phenylamino)-N-(1,7,7-trimethylbicyclo[2.2.1]heptan-2-yl)pyrimidine-4-carboxyamide; and
137) 2-(3-(4-isopropylpiperazin-1-yl)phenylamino)-N-(1,7,7-trimethylbicyclo[2.2.1]heptan-2-yl)pyrimidine-4-carboxyamide.

4. A method for preparing a compound of formula 1, comprising
1) reacting a compound of formula 2 with NaNO₂ in a solvent to obtain a compound of formula 3;
2) reacting the compound of formula 3 with a halogenating agent to obtain a compound of formula 4;
3) reacting of the compound of formula 4 and a compound of formula 5 in a solvent under microwave irradiation to obtain a compound of formula 6;
4) dissolving the compound of formula 6 in an alkali hydroxide, refluxing the resulting mixture, and adding a strong acid thereto until pH of the mixture becomes 3, to obtain a compound of formula 7; and
5) subjecting the compound of formula 7 an amidation reaction with a compound of formula R₂R₃NH in a solvent in the presence of a coupling agent to obtain the compound of formula 1: 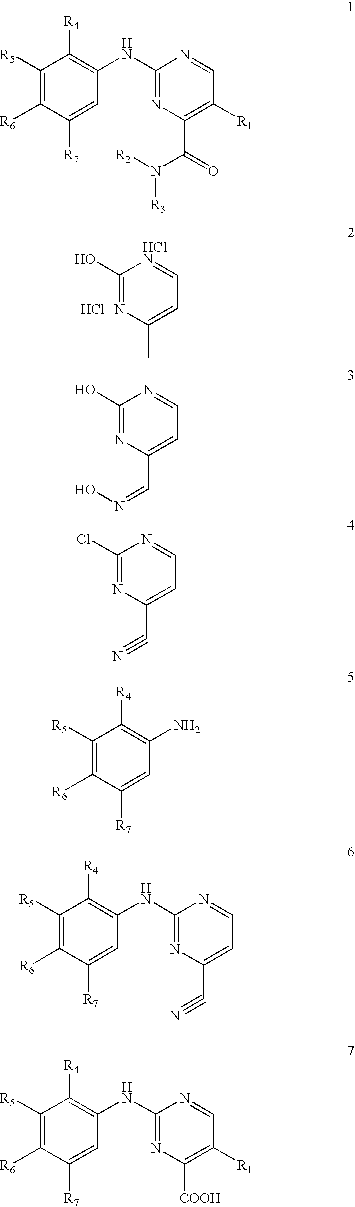
wherein, R₁ is hydrogen; and R₂, R₃, R₄, R₅, R₆ and R₇ have the same meanings as defined in claim 1.

5. A method for preparing a compound of formula 1, comprising
1) allowing a compound of formula 8, thionylchloride (SOCl₂) and methanol to react in a solvent to obtain a compound of formula 9;
2) dissolving the compound of formula 9 in a solvent, and adding an oxidizing agent thereto to obtain a compound of formula 10;
3) subjecting the compound of formula 10 and a compound of formula 5 to a reaction under microwave irradiation to obtain a compound of formula 11;
4) dissolving the compound of formula 11 in an alkali hydroxide, refluxing the mixture, and adding a strong acid thereto until pH of the mixture becomes 3, to obtain a compound of formula 7; and
5) amidating the compound of formula 7 with a compound of formula R₂R₃NH in a solvent in the presence of a coupling agent to obtain the compound of formula 1: 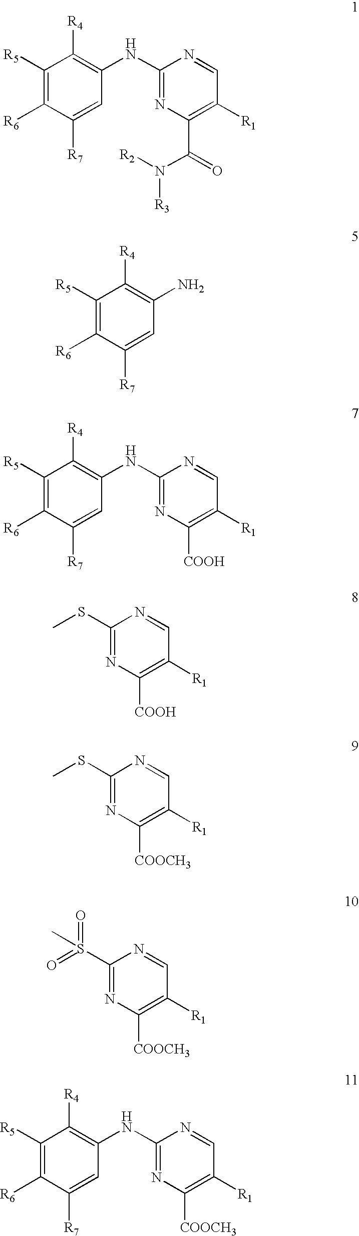
wherein, R₁ is hydroxy, halogen, C₁₋₂ alkyloxy or C₁₋₂ alkyl; R₃ is hydrogen; and R₂, R₄, R₅, R₆ and R₇ have the same meanings as defined in claim 1.

6-7. (canceled)

8. A method for preparing a compound of formula 23a, comprising
1) reacting a compound of formula 16a with p-chlorobenzyl chloride in a solvent to obtain a compound of formula 17a;
2) reducing the compound of formula 17a to obtain a compound of formula 18a;
3) subjecting the compound of formula 18a and a compound of formula 4 to a reaction under microwave irradiation to obtain a compound of formula 19a;
4) dissolving the compound of formula 19a in an alkali hydroxide, refluxing the mixture, and adding a strong acid thereto until pH of the mixture becomes 3 to obtain a compound of formula 20a;
5) amidating the compound of formula 20a with a compound of formula R₂R₃NH in a organic solvent in the presence of a coupling agent to obtain a compound of formula 21a;
6) removing the p-chlorobenzyl group from the compound of formula 21a to obtain a compound of formula 22a; and
7) alkylating the compound of formula 22a in the presence of a base to obtain a compound of formula 23a: 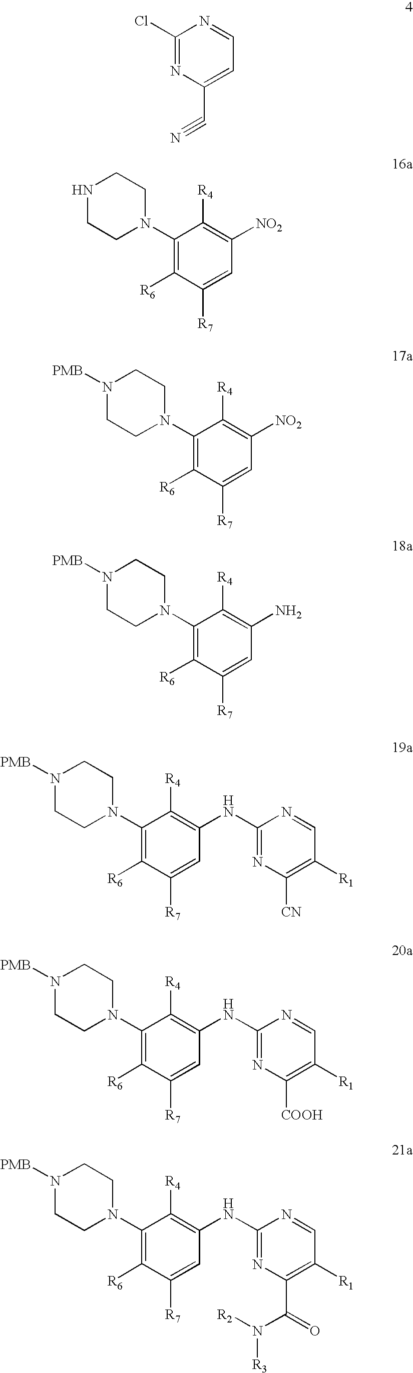 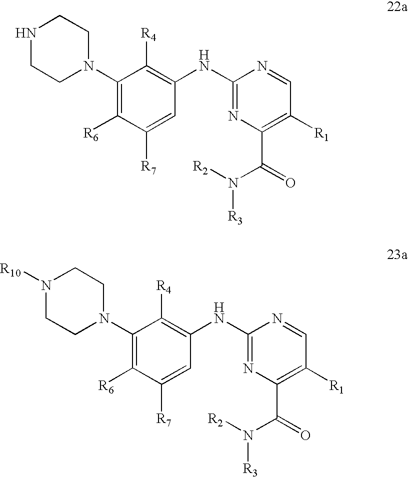
wherein, R₁ is H; R₂, R₃, R₄, R₆ and R₇ have the same meanings as defined in claim 1; and R₁₀ is C₁₋₆ alkyl.

9. A method for preparing a compound of formula 23b, comprising
1) reacting a compound of formula 16b with p-chlorobenzyl chloride in a solvent to obtain a compound of formula 17b;
2) reducing the compound of formula 17b to obtain a compound of formula 18b;
3) subjecting the compound of formula 18b and a compound of formula 4 to a reaction under microwave irradiation to obtain a compound of formula 19b;
4) dissolving the compound of formula 19b in an alkali hydroxide, refluxing the mixture, and adding a strong acid thereto until pH of the mixture becomes 3 to obtain a compound of formula 20b;
5) amidating the compound of formula 20b with a compound of formula R₂R₃NH in a organic solvent in the presence of a coupling agent to obtain a compound of formula 21b;
6) removing the p-chlorobenzyl group from the compound of formula 21b to obtain a compound of formula 22b; and
7) alkylating the compound of formula 22b in the presence of a base to obtain a compound of formula 23b: 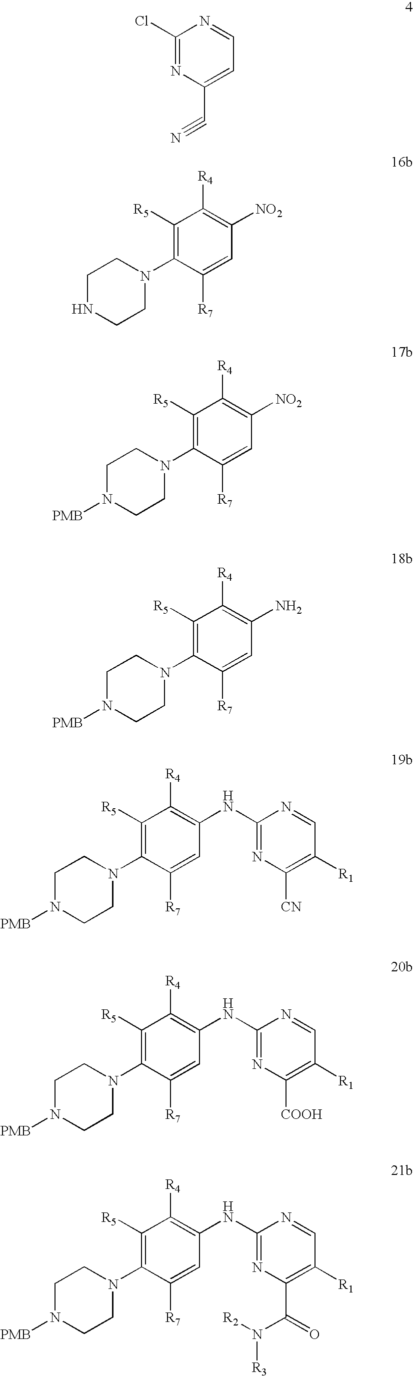 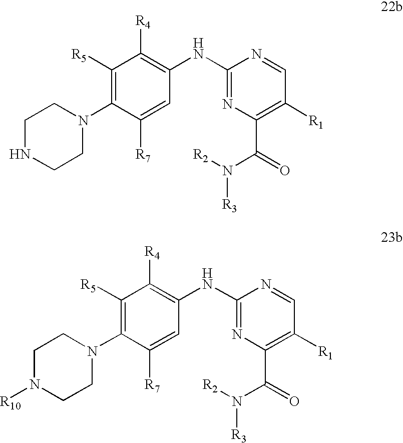
wherein, R₁ is H; R₂, R₃, R₄, R₆ and R₇ have the same meanings as defined in claim 1; and R₁₀ is C₁₋₆ alkyl.

10. A composition for inhibiting protein kinase comprising the compound of formula 1 of claim 1, or a pharmaceutically acceptable salt, hydrate, solvate or isomer thereof as an active ingredient.

11. The composition of claim 10, wherein the protein kinase is selected from the group consisting of glycogen synthase kinase 3 (GSK), aurora kinase, extracellular signal-regulated kinase (ERK), protein kinase B (AKT), cyclin dependent kinase (CDK), p38 (protein 38) mitogen-activated protein kinase (MAPK) and c-Jun-N-terminal kinase (JNK).

12. The composition of claim 11, wherein the protein kinase is selected from the group consisting of aurora kinase, glycogen synthase kinase 3 (GSK), cyclin dependent kinase (CDK) and c-Jun N-terminal kinase (JNK).

13. An anticancer composition comprising the compound of formula 1 of claim 1, or a pharmaceutically acceptable salt, hydrate, solvate or isomer thereof as an active ingredient.
